(19) United States
(12) Patent Application Publication
(10) Pub. No.: US 2003/0204415 A1
(43) Pub. Date: Oct. 30, 2003

(54) Medical data and medication selection and distribution system

(76) Inventor: Calvin Knowlton, Cinnaminson, NJ (US)

Correspondence Address: MORGAN, LEWIS & BOCKIUS LLP, 1701 MARKET STREET, PHILADELPHIA, PA, 19103-2921, US

(21) Appl. No.: 10/217,277

(22) Filed: Aug. 12, 2002

Related U.S. Application Data
(60) Provisional application No. 60/377,040, filed on Apr. 30, 2002.

(51) Int. Cl. G06F 17/60; A61B 5/00

(52) U.S. Cl. 705/2; 600/300

(57) ABSTRACT

A system and method for selecting a medication therapy for a patient. The system receives input data relating to a patient profile for the patient where the patient profile includes at least one of an objective attribute and a subjective attribute. The system compares the patient profile of the patient to an iterative patient database that includes a plurality of the patient profiles for a plurality of patients where each of the plurality of patients having at least one of the objective attribute and the subjective attribute that is similar to at least one of the objective attribute and the subjective attribute of the patient profile for the patient. The system selects the medication therapy for the patient based on the comparison.

### CROSS REFERENCE TO RELATED APPLICATIONS

This application claims priority to provisional patent application No. 60/377,040, entitled “Medical Data and Medication Selection and Distribution System”, filed Apr. 30, 2002, incorporated herein by reference.

### FIELD OF THE INVENTION

The present invention relates to systems and methods for providing a medication selection and distribution network system. In particular, the present invention relates to systems and methods for providing predictive models in medication selection and distribution system.

### BACKGROUND OF INVENTION

In general, there are many issues surrounding the healthcare system. One of the most serious issues is misusing prescription medications. In the United States alone, for instance, there is serious, unintentional, misuse of prescription medications that causes tens of thousands of preventable deaths per year, and rampant morbidity. While there are several causes behind such a misuse of medication, the most prevalent cause relates to the current medication selection and distribution system. That is, many medication misuse situations stem from making an inappropriate selection and/or distribution of medication. Considering the dramatic increase in the number of new drugs and the complexity of such, the increase of medication misuse is not entirely surprising.

In medication distribution and selection systems, physicians prescribe, pharmacists dispense, and nurses administer and care for patients. Many healthcare providers have computerized information systems, which are typically stand-alone systems. Thus, a particular prescription decision may be at the mercy of one individual prescriber's clinical judgment, which may or may not reflect the most appropriate clinical judgment. This is further complicated by the fact that patients frequently have multiple physicians, and often, multiple pharmacies that, more likely than not, do not know what the others are prescribing or dispensing.

There are other factors that complicate the situation even further: the “externalities” that represent non-medical factors. Some of these externalities include, for instance, heavy marketing and promotion of drug manufacturers, financial decisions made by non-medical professionals, etc.

Thus, it is believed that there is a need for systems and methods for medication selection and distribution that can be used to reduce the number of medication misuses. In particular, it is believed that there is a need to provide a means to facilitate communication among caregivers (i.e., healthcare providers) and between caregivers and patients. It is also believed that there is a need for caregivers to provide access to patients. It is further believed that there is a need for systems and methods that can be used to select appropriate medication therapy for a patient. It is also believed that there is a need for systems and methods that can be used to distribute the medication therapy for a patient.

### SUMMARY OF PREFERRED EMBODIMENTS OF THE INVENTION

In one embodiment, the present invention is directed to a system and method for selecting a medication therapy for a patient. Preferably, the system receives input data relating to a patient profile for the patient where the patient profile includes at least one of an objective attribute and a subjective attribute. Preferably, the system compares the patient profile of the patient to patient data that includes a plurality of the patient profiles for a plurality of patients where each of the plurality of patients includes at least one of the objective attribute and the subjective attribute that is similar to at least one of the objective attribute and the subjective attribute of the patient profile for the patient. Preferably, the system identifies one or more medication therapies for the patient based on the comparison.

In one embodiment, the present invention is directed to a system for selecting a medication therapy of a patient. In one aspect, the system includes an iterative database that stores a plurality of patient profiles for a plurality of patients where each of the plurality of patient profiles includes at least one of an objective attribute and a subjective attribute. In another aspect, the system further includes a processor that receives input data relating to the patient profile of the patient, compares the patient profile including at least one of the objective attribute and the subjective attribute to a subset of the plurality of patient profiles including at least one of the objective attribute and the subjective attribute that is similar to at least one of the objective attribute and the subjective attribute of the patient profile, and selects the medication therapy for the patient based on the comparison.

In one embodiment, the present invention is directed to a network system for selecting and distributing a medication therapy for a patient. In one aspect, the network system includes an iterative database that stores a plurality of patient profiles for a plurality of patients where each of the plurality of patient profiles includes at least one of an objective attribute and a subjective attribute. In another aspect, the network system also includes a wireless device that transmits input data including at least one of the objective attribute and the subjective attribute for the patient profile of the patient. Preferably, the wireless device also receives output data. In yet another aspect, the network system further includes a processor that receives the input data including the profile of the patient, compares the patient profile of the patient to a subset of the plurality of the patient profiles where each of the patient profile from the subset includes at least one of the objective attribute and the subjective attribute that is similar to at least one of the objective attribute and the subjective attribute of the patient profile of the patient, selects the medication therapy for the patient based on the comparison, and transmits the output data that includes the medication therapy for the patient.

In one embodiment, the present invention is directed to a system and method for selecting a medication therapy for a patient. Preferably, the system presents a graphical user interface to a caregiver of the patient, and receives via the graphical user interface input data relating to a patient profile of the patient from the caregiver where the patient profile includes at least one of an objective attribute and a subjective attribute. Preferably, the system compares the patient profile of the patient to patient data that includes a plurality of patient profiles for a plurality of patients where each of the patient profiles includes at least one of the objective attribute and the subjective attribute that is similar to at least one of the objective attribute and the subjective attribute of the patient. Preferably, the system predicts outcomes of administering the medication therapy for the patient based on the comparison, and displays the outcomes to the caregiver via the graphical user interface.

### BRIEF DESCRIPTION OF THE DRAWINGS

FIG. 1 a diagram of one embodiment showing a medication selection and distribution network system of the present invention.

FIG. 2 a block diagram showing various components of the computer system of the present invention.

FIG. 3 is a block diagram showing the components of an integrated, on-line interactive system of the present invention.

FIG. 4A is a flowchart illustrating one embodiment of functions of a network system in accordance of the present invention.

FIG. 4B is a flowchart illustrating one embodiment of the present invention.

FIG. 4C is a flowchart illustrating another embodiment of the present invention.

FIGS. 5A-5F show flowcharts illustrating one embodiment of the present invention using an on-line interactive system of the present invention.

FIGS. 5G-5Y depict exemplary web pages showing the embodiment of FIGS. 5A-5F.

FIGS. 6A-6F show flowcharts illustrating another embodiment of present the invention where caregivers use an on-line interactive system of the present invention.

FIGS. 6G-6T depict exemplary web pages of the embodiment of FIGS. 6A-6F.

FIG. 7A shows a flowchart illustrating yet another embodiment of the invention where caregivers use on-line interactive system of the present invention.

FIGS. 7B-7F depict exemplary web pages of the embodiment of FIG. 7A.

FIG. 8 shows a flowchart illustrating yet another embodiment of the invention where caregivers use on-line interactive system of the present invention.

FIG. 9A shows a flowchart illustrating yet another embodiment of the invention where caregivers use on-line interactive system of the present invention.

FIGS. 9B-9E depict exemplary web pages of the embodiment of FIG. 9A.

FIGS. 10A-10C show flowcharts illustrating yet another embodiment of the invention where caregivers use on-line interactive system of the present invention.

FIGS. 10D-10K depict exemplary web pages of the embodiment of FIGS. 10A-10C.

FIG. 11A shows a flowchart illustrating yet another embodiment of the invention where caregivers use on-line interactive system of the present invention.

FIGS. 11B-11J depict exemplary web pages of the embodiment of FIG. 11A.

FIG. 12A shows a flowchart illustrating yet another embodiment of the invention where caregivers use on-line interactive system of the present invention.

FIGS. 12B-12E depict exemplary web pages of the embodiment of FIG. 12A.

FIG. 13A shows a flowchart illustrating yet another embodiment of the invention where caregivers use on-line interactive system of the present invention.

FIGS. 13B-13D depict exemplary web pages of the embodiment of FIG. 13A.

FIG. 14 shows a flowchart illustrating yet another embodiment of the invention where caregivers use on-line interactive system of the present invention.

FIG. 15 shows a flowchart illustrating yet another embodiment of the invention where caregivers use on-line interactive system of the present invention.

FIG. 16 shows a flowchart illustrating yet another embodiment of the invention where caregivers use on-line interactive system of the present invention.

FIG. 17 shows a flowchart illustrating yet another embodiment of the invention where caregivers use on-line interactive system of the present invention.

FIG. 18 shows a flowchart illustrating yet another embodiment of the invention where caregivers use on-line interactive system of the present invention.

### DETAILED DESCRIPTION OF THE PREFERRED EMBODIMENTS

Reference will now be made in detail to the preferred embodiments of the present invention, examples of which are illustrated in the accompanying drawings. Wherever possible, the same reference numbers will be used throughout the drawings to refer to the same or like parts and steps. The accompanying figures are illustrative, but not limiting, of the present invention.

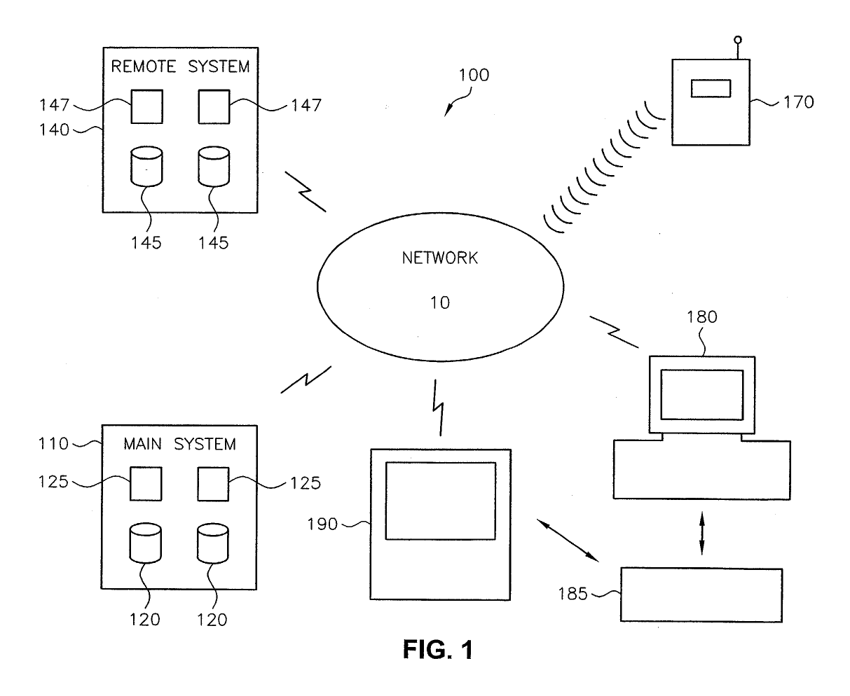

In accordance with one aspect of the present invention, a novel system and method for providing medication management are provided. One embodiment of the present invention relating to a medication management, and medication selection and distribution through a network system, is illustrated in FIG. 1. Network system 100 facilitates providing effective patient care by allowing caregivers to conduct traditional patient medication care activities, such as acquiring and using pertinent patient and medication information, prescribing and distributing medications, and monitoring patient medication uses, at any time and any place using any computer devices and the like such as a personal computer or wireless Internet access device including hand-held devices.

In particular, network system 100 can be used, among other things, to integrate a decentralized medication selection and dispensing processes into a shared, centralized, controlled environment. More specifically, network system 100 serves to integrate the collection process of patient data, medication trial data, actual patient treatment outcome data, and other relevant clinical data by bringing caregivers into a shared, centralized, controlled environment. Using the integrated collection of patient data and medication data, network system 100 can be used to improve medication prescribing and dispensing decisions. The improved decisions, in turn, promote, among others, the safety and efficacy of patient medication uses.

As discussed in detail below, network system 100 includes specialized databases that include patient profiles and other evidenced-based palliative pharmacotherapy data that enable caregivers to find answers to their questions. More in particular, network system 100 allows the caregivers to predict probable outcomes of using a particular medication to a particular patient having a particular medical indication. In other words, using network system 100, caregivers can predict, with evidenced-based accuracy, what outcome is expected when a particular treatment or medication therapy is administered to a particular patient having a particular medical condition. It should be noted that, in accordance with the present invention, the term “caregiver” means any authorized user who has access privilege over network system 100. In general, caregivers would typically include prescribers, physicians, nurses, pharmacists, hospice caretakers, and in certain cases, patients. In some instances, only caregivers who are authorized to prescribe medication or medical attention (i.e., physicians who prescribe medications or medical attention) will have access to use certain aspects of the present invention.

As shown in FIG. 1, it should be apparent that, using network 10, caregivers can remotely access network system 100 securely in real time by using any electronic communication media such as personal computer 180 or Personal Digital Assistant 170. Those skilled in the art will understand that any user interface may be used to input data, including but not limited to a keyboard, mouse, and other peripheral inputting devices of a computer system. Note that personal computer 180 or any other electronic communication media can be connected to displaying devices such as a monitor or printing devices such as printer 185. Using printer 185, all exchanged information over network 10 can be printed in hard copies. Personal computer 180 can be located anywhere including caregiver's home or office. Also, portable, wireless Internet access devices such as Personal Digital Assistant 170 can be used to access network system 100 remotely via network 10. Once connected to network 10, Personal Digital Assistant 170 or personal computer 180 can be used to connect to main system 110 and remote system 140. Note that network 10 can be any type of network systems such as a Local Area Network, Wide Area Network, or a global network such as the Internet.

Also included in network system 100 is an automated medication prescription device such as Access to Medication (ATM) machine 190, which is described further below. ATM 190 is a computing device that is capable of being operatively connected to network system 100 for exchanging information via network 10. Like personal computer 180, ATM 190 comprises processing means for sending, receiving, and processing information via network 10. Furthermore, ATM 190 can also be coupled to a printing device such as printer 185 for printing hard copies, or include a printing device integrated within ATM 190. Those skilled in the art will understand that network system 100 may receive communication signals over any suitable medium such as twisted-pair wire, co-axial cable, fiber optics, radio-frequencies, and so forth.

Network system 100 includes main system 110, which, as described more in detail below, includes one or more processors 125. Main system 110 can be any commercially available computer system such as a server, minicomputer or microcomputer, mainframe, and the like. Main system 110 further includes one or more specialized databases 120 for storing, among other things, patient data, medical reference data, and Medical Use Guidelines (MUGs) data. As shown, one or more processors 125 may be used in connection with executing a number of different computer programs or software applications in carrying out the methods of the present invention. Main system 110, in accordance with one aspect of the present invention, is preferably located at a main facility such as a central data management facility.

In one embodiment of the present invention, main system 110 is located at a centralized contact center (or call center) equipped with on-site pharmacists and medication inventories. Having all relevant medication systems and tools including, pharmacists, medication supplies, and support staff, the contact center can facilitate all traditional medication management functions from prescribing to dispensing from one facility. In one embodiment, as described further below, a contact center equipped with main system 110 can streamline all traditional medication processes such as suggesting medications, ordering of refills, custom medications, or door-to-door delivery for patients.

Network system 100 further includes one or more remote systems 140, which are operatively connected to main system 110 via a global network such as the Internet. As shown in FIG. 1, remote system 140 is connected to network system 100 via network 10. Remote system 140 is similar to main system 110. Remote system 140 can be a computer system having one or more processors 147 and one or more specialized databases 145. In accordance with one embodiment of the present invention, remote system 140 is preferably located at one or more medication care facilities such as a hospital, pharmacy, hospice, and medication dispense center.

It should be apparent from the foregoing description that processors 125 can access databases 120 using local links such as a bus system. Using network 10, processors 125 may also access databases 145 of remote system 140. Like databases 120 of main system 110, databases 145 may store at least one of patient data, medical reference data, and MUGs data. Thus, processors 125 may access all files included in databases 145 via network 10 and look up data, in addition to data stored in databases 120, as needed in carrying out methods of the present invention. Likewise, processors 147 can access databases 145 within remote system 140 using its local links, or alternatively or additionally, processors 147 can access databases 120 of main system 110 via network 10. Of course, processors 147 of a remote system 140 can also access databases 145 of another remote system 140 via network 10.

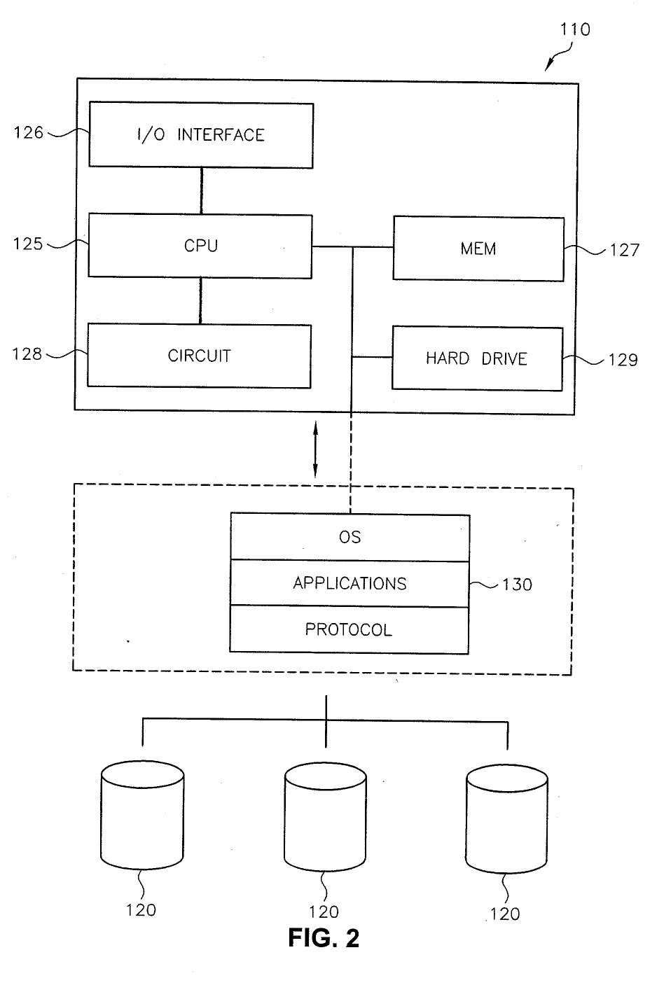

FIG. 2 shows one embodiment of components of main system 110, which can be any commercially available computer system, such as a server, mainframe, or microcomputer. As illustrated earlier, main system 110 includes at least one processor (or CPU) 125. Processor 125 is operable with, among other things, a main memory 127, an input/output (I/O) device 126, and such well-known support circuits 128 as power supplies, clocks, caches, displays, and the like. I/O device 126 receives and transmits electrical signals corresponding to an electrical signal that passes over network 10. Main memory 127 includes instructions that processor 125 executes to facilitate the processing, storage, transfer, and control of data transfer and storage. Instructions in memory 127 are in the form of program code. Main system 110 further comprises hard drive 129 for storing computer programs or application software, in accordance with one aspect of the present invention. Operating system software, application software, and other intelligent protocols or modules, collectively referred to as program modules 130 are stored in hard drive 129 and main memory 127 of main system 110. Using instructions of modules 130, processors 125 communicate with databases 120. As discussed in detail below, using modules 130 and databases 120, in accordance with the present invention, novel ways of collecting and storing patient data and medication data, accessing and using patient data and medication data, and predicting outcomes of administering selected medications to patients are provided.

Like main system 110, one or more remote systems 140, and other computer systems such as personal computer 180 that interface with network 10, may also be such servers or microcomputers capable of communicating over a computer network. Accordingly, program modules 130 may also be located in remote systems 140, or personal computer 180, and provide the same or similar functionality and utility as main system 110. Those of ordinary skill in the art will recognize network system 100 (shown in FIG. 1) may connect to any number of additional computer systems that are capable of providing the functions of main system 110.

Referring again to FIG. 1, main system 110 includes one or more databases 120 that facilitate carrying out the methods of the present invention. Databases 120 include, as described further below, a patient database. The patient database comprises information relating to patients profiled in network system 100. Preferably, the patient database comprises information from all patients ever profiled in network system 100 and each patient profile is comprehensive, i.e., it includes all relevant patient and/or medication records. Thus, the term “profiling” is used to describe the process of recording what a patient is taking (i.e., what was already prescribed and dispensed as well as the over-the-counter products). Using network system 100, in accordance with one embodiment of the present invention, the source of the information that forms the basis of patient profile comes from one or more sources including the patient, pharmacy, hospice, hospital, lab, nurses, physicians, etc. The patient database of the present invention is preferably comprehensive.

As described in detail below, the comprehensive patient database means, as used in this disclosure, the patient database that includes information representing both objective attributes and subjective attributes. In accordance with the present invention, the term “objective” is used to refer to those attributes that are readily observable or measurable, and that can be easily compared among all patients. The objective attributes of the patient database include, for instance, the patient's gender, which can be easily compared from one patient to another. The term “subjective” is used to refer to those attributes that may not be equally applicable to all patients. The subjective attributes define, for instance, a patient's pain level, mobility, or personal satisfaction with a particular treatment or medication. The subjective attributes may also include an individualized result of treatment—e.g., the measure of how well a particular medication worked when administered to a patient having a particular symptom. These subjective attributes may not be easily compared from one patient to another. In other words, the subjective attributes define a “quality of life” of a patient by quantifying otherwise immeasurable factors. The subjective attributes may include, among others, subjective attributes obtained using objective tests such as the Wisconsin Pair Inventory.

Accordingly, the patient database includes, among others, objective patient profile attributes such as patient's demographic profile and medical history, all tailored to each patient. Medical history includes all pertinent medical information such as the patient's treating physician information, medication history including current prescription and over-the-counter medications, lab results, generic history, hospital and hospice records, recent diagnosis, existing allergy, etc. Medical history may also include a physician's (or any other qualified caretaker's) observation of using a particular medication on a patient. Demographic profile includes all other relevant information such as patient's age, contact information, race, geographic information, etc. The patient database also includes the subjective patient profile attributes such as the pain level indicated by the patient and the pain level diagnosed by a treating physician. The subjective attributes further include a patient's opinion, such as one's satisfaction, regarding using a particular medication. In should be apparent from the foregoing description that the patient database of the present invention represents a unique combination of both patient inputs and non-patient inputs.

Databases 120 also include a general medical reference database. The medical reference database is a database containing relevant medication and therapeutic information. In one embodiment of the present invention, the medical reference database includes First DataBank (FDB) database, which includes descriptive, economic and clinical information relating to over 200,000 drug products. As noted earlier, databases 120 may further include a Medical Use Guidelines (MUGs) database. MUGs database, in accordance with one aspect of the present invention, includes evidenced-based and clinician-based, clinical trial results of selected medications that serve as a guide for prescribing medication for certain medical indications.

MUGs database further includes peer-reviewed, step-care protocols relating to all relevant aspects of selected medications. The relevant aspects include, among other things, the efficacy, safety including any side effects, long term effect, cost information, and patient's unique and general response or reaction to selected medications. For instance, some of the protocols included in MUGs database may show the efficacy and safety of medications and treatments relating to Congestive Heart Failure, End Stage Renal Disease, and Palliative Sedation.

Furthermore, MUGs database can be organized into multiple representations. For instance, MUGs database can organize selected medications based on their efficacy relating to particular indications. In one embodiment, selected medications within the protocols of MUGs database are sorted by diagnosis coverage code in the index. In yet another embodiment, a brand and generic list of medications of over 75 compounds and an injectable medications list are included in MUGs database. It should be noted that MUGs database is dynamic; it is constantly updated by a medical professional committee to reflect new findings and guidelines relating to selected medications.

In accordance with one aspect of the present invention, databases 120, 145 include a database system using a query language such as Structured Query Language (SQL) database. SQL database can be used to extract data from databases 120, 145. SQL database system facilitates the utility and functionality of network system 100 since, in one embodiment of the present invention, databases 120 and 145 are spread out over two or more computer systems over network system 100. Using SQL database system allows multiple caregivers on network system 100 to simultaneously access databases 120, 145.

In accordance with another aspect of the present invention, databases 120, 145 are iterative. That is, databases 120, 145 may comprise an iterative database of empirical data on the effects of medication therapies on a plurality of patients whereby the patients can be stratified based on patient profile parameters, including subjective and/or objective attribute. Databases 120, 145 are updated after each access.

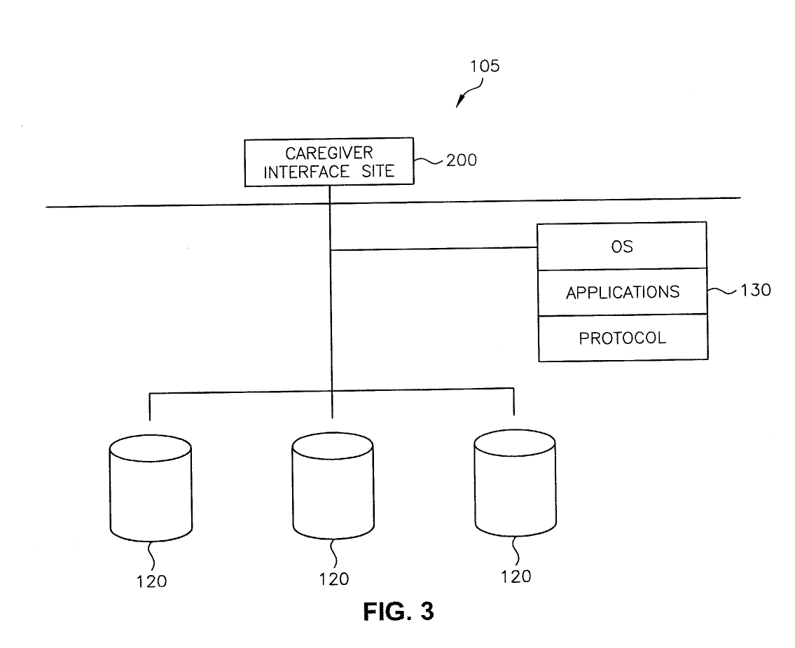

FIG. 3 shows one aspect of the present invention illustrating a novel system and method of using network system 100 called Predictive Pharmacotherapy Outcome System (PPOS) 105. PPOS 105, shown in FIG. 3, comprises major components of main system 110. Thus, PPOS 105 includes program modules 130 and databases 120. In one embodiment as shown in FIG. 3, PPOS 105 represents a logical construction of an integrated, online caregiver interactive interface 200 using main system 110 according to the present invention. As shown, PPOS 105 includes caregiver interface site 200. Caregiver interface site 200 represents one aspect of active server pages (ASP) that are accessed using an interactive programming language or forum such as an Intranet or Extranet site, or query program such as Microsoft's Analysis Services. In one embodiment, caregiver interface site 200 comprise a dynamically created web page site that utilizes Object Linking and Embedding (OLE) or Component Object Model (COM) technologies such as ActiveX scripting—usually VB Script or Jscript code.

Caregiver interface site 200 is used in many aspects to carry out the methods of the present invention. For instance, caregiver interface site 200 can be used to facilitate information exchange between and among caregivers, main system 110, and remote system 140. Caregivers, using a network browser on their personal computers or wireless, hand-held Internet devices, request caregiver interface site 200, and then main system 110 generates a page with web-based authoring tools such as HTML (or XML) code and sends it back to the browser. In effect, in accordance with one aspect of the present invention, caregiver interface site 200 represents a centralized server site for caregivers to, among other things, conduct all relevant communications between and among each other, main system 110, and remote system 140. Thus, PPOS 105, while using caregiver interface site 200, is used to carry out multiple embodiments of the present invention.

As noted earlier, in one embodiment network system 100 facilitates providing effective patient care by allowing caregivers to conduct traditional patient medication care activities, such as acquiring and using all pertinent patient information, prescribing and distributing medications, and monitoring patient medication uses, etc., electronically at any time and any place using any computer devices and the like such as a personal computer or wireless Internet access device including hand-held devices. Caregiver interface site 200 provides one aspect of the present invention that facilitates interactions between caregivers and network system 100. Thus, caregivers use caregiver interface site 200 to submit input data to network system 100 and to receive output data from network system 100.

For instance, in the embodiment shown in FIG. 1, caregivers log in to main system 110 using personal computer 180 via network 10. Once logged in to main system 110, a caregiver will be directed to caregiver interface site 200 as shown in FIG. 3. Caregiver interface site 200 can be used by authorized caregivers to access sensitive and/or privileged information stored in databases 120, 145. Therefore, in one embodiment of the present invention, after a caregiver has logged into main system 110 via network 10, it is necessary to enter a caregiver identification number along with a password. Thereafter, with the aid of program modules 130 of main system 110 or remote system 140, the caregiver can access the contents of databases 120, 145.

It should be apparent that the present invention provides a secure environment for these caregiver interactions. In all embodiments of the present invention, the system includes software and hardware that can be used to secure all data and transactions in the present invention. For example, all data and information transmitted and received using network system 100, and stored in main system 110 or remote system 140 may be encrypted and/or password (or access code) protected. Further, any user's (e.g., caregiver's) access may be restricted to certain data and certain information by appropriate password (or access code) and/or encryption protection.

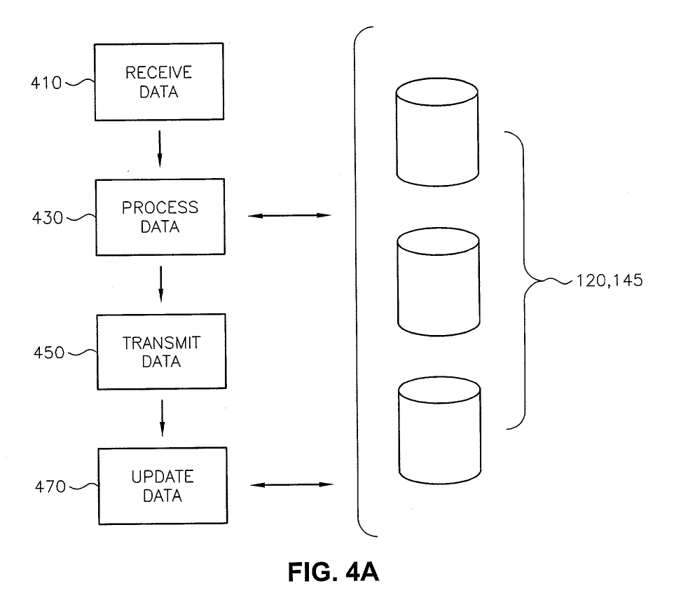

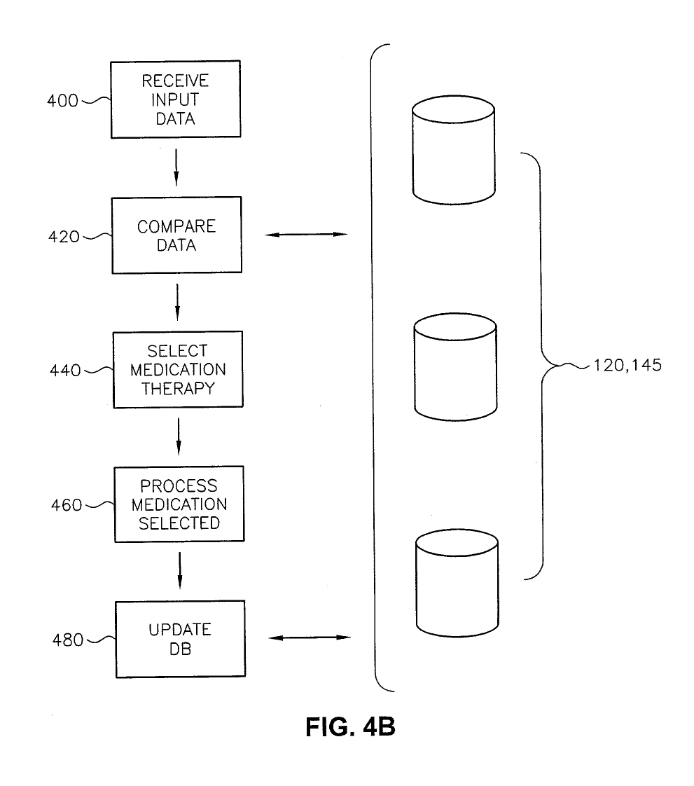

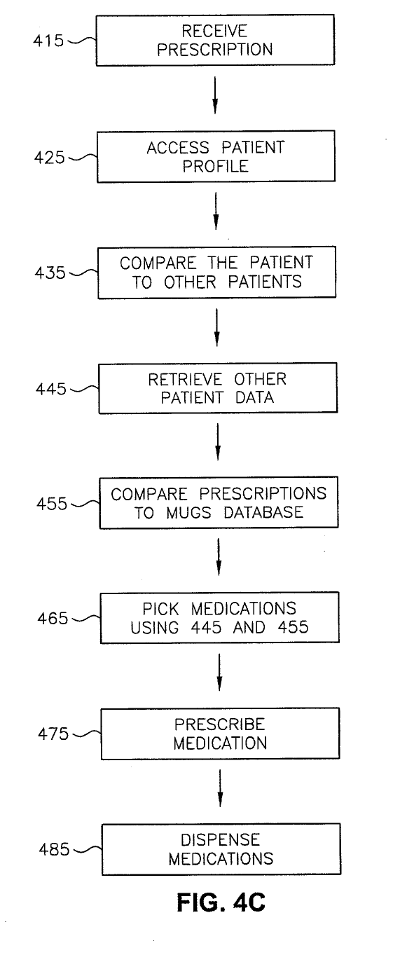

FIG. 4A shows a flowchart illustrating the overall function of network system 100 in accordance with one aspect of the present invention. As shown in FIG. 4A, a caregiver may transmit data over network 10. In step 410, input data is received by main system 110 or in some selected cases, by remote system 140. Input data includes, among other things, one or more records relating to patients and or medications. Input data also includes instructions or commands with respect to records. Input data is processed per instructions or commands in step 430. That is, using program modules 130, main system 110 reads the instructions or commands and processes records received. The instructions include algorithms, which are used to compare an individual patient profile and medical indication of that patient to the profiles of all other patients or a select set of patients in databases 120, 145. Thus, databases 120, 145 are referenced during the data processing step.

After processing input data, output data is transmitted to the caregiver in step 450. Output data contains, among other things, information sought by the caregiver. In step 470, databases 120, 145 are updated reflecting the current transaction.

In accordance with one aspect the present invention, using databases 120, 145, including patient records database, medical reference database, and MUGs database, a novel way of predicting medication outcomes using PPOS 105 is provided to caregivers. As noted, PPOS 105 represents a novel system and method of the present invention. Using network system 100, caregivers can make informed decisions relating to prescribing medications. Network system 100 allows caregivers to use PPOS 105 and make better, informed prescription judgment for a patient by enabling caregivers to query “like patients” and then use the information to predict outcomes for the patient.

FIG. 4B shows a flowchart illustrating a novel method, in accordance with one aspect of the present invention, to improve medication selection and dispensing by allowing caregivers to predict with improved accuracy and reliability the outcomes of using a given medication. That is, using PPOS 105, caregivers can predict the probable outcome of administering a particular medication or treatment to a particular patient showing a particular indication. In step 400, input data including one or more patient parameters are received. The patient parameters include pertinent information relating to a patient, including but not limited to, one or more objective and subjective attributes and/or outcomes of the patient. The objective and subjective attributes, as noted earlier, may include one or more of patient's medical history, demographic information, medication history, allergies, pharmacogenomic data, pain level, etc. The medical history may include the patient's current disease and stage of the disease. As noted earlier, the patient parameter having at least one objective and/or subjective attribute can be submitted to network system 100 using any computing means such as personal computer 180 or Personal Digital Assistant 170. For instance, using personal computer 180, a caregiver may connect to main system 110 via network 10. Thereafter, the caregiver can access PPOS 105 via caregiver interface site 200.

As discussed further below, using databases 120, 145, one or more patient parameters are then compared to patient data of other patients who have substantially similar profiles and/or substantially similar medication indications as the patient, in step 420. It should be noted that, as described above, databases 120, 145 may comprise an iterative database of empirical data on the effects of medication therapies on a plurality of patients whereby the patients can be stratified based on patient profile parameters, including subjective and/or objective attribute. It should also be noted that, as described further below, in accordance with one aspect of the present invention, the term “medical condition” refers to any cause, pathology, treatment or issue of an attack of disease, that which points out, or that which serves as a guide or warning. As such, unless otherwise specified, the term is used interchangeably with the term “diagnosis,” which generally indicates the determination of the nature of a case of disease.

The comparison (i.e., of the patient profile to other patient profiles) is used to predict probable outcomes (e.g., efficacy and safety), of administering one or more selected medications and/or treatments to the patient. In one embodiment, to predict the outcomes of administering a selected medication to the patient, PPOS 105 uses databases 120 and program modules 130 to assess the genetic basis for differences in medication efficacy and toxicity, and PPOS 105 further assesses and tracks genes that govern the patient's likely response to medication therapy. In this embodiment, PPOS 105 defines the contributions of genetic differences in medication disposition, and uses the results to significantly improve the safety and efficacy of medication therapy through genetically guided, individualized treatments for the patient.

While carrying out one aspect of the present invention, PPOS 105 specifically compares the patient's current profile, e.g., objective and/or subjective attributes such as demographic information, specific medical history including recent procedures and medications administered to the patient, and classified patients according to medical indications represented in a coding system such as International Classification of Diseases system (i.e., ICD-9), with profiles of other similar patients and their medical profiles along with their outcomes of using different treatments and medications. Using the comparison, PPOS 105 derives predictions that illustrate probable outcomes of using a selected medication to the patient within the ICD-9 coding classification. Thus, PPOS 105's predictions are patient-specific. That is, the predictions are not based on using the relevant medication information such as the efficacy and safety of a particular drug according to a clinician derived treatment protocol such as MUGs; rather, the predictions are primarily based on the findings of the medication and its specific results (e.g., its overall effectiveness) achieved on other patients having profiles that are substantially similar to the patient's profile. In step 440, using the comparison from step 420, the most appropriate medication and/or other treatment is selected. The following example illustrates the process of predicting the outcomes of selected medications and to select one or more medications based on the outcome.

Assuming input data that includes a patient parameter of a subject patient showing a medical indication of Congestive Heart Failure are submitted to main system 110 via network 10, PPOS 105 then selects all medications that are proven to effectively treat Congestive Heart Failure. PPOS 105 retrieves this information mainly from databases 120. Alternatively or additionally, PPOS 105 may retrieve the information from databases 145 via network 10. As noted earlier, databases 120, 145 contain MUGs database, which includes all pertinent, evidence-based trial results of selected medications.

Upon identifying the list of selected medications that are proven effective against Congestive Heart Failure, PPOS 105 further searches databases 120, 145 for all patients who have been or are currently being treated using the selected medications. All patients selected may have been treated or are being treated using the selected medications for different purposes as opposed to being used for Congestive Heart Failure. Thus, the list of patients can be adjusted to include only those patients who used or are using the treatments for Congestive Heart Failure. Those patients who have used or are using the selected medications and/or treatments for different purposes can be effectively screened out.

In one embodiment, however, the screened out patients from the above step may be included for further analysis. That is, the screened out patients (i.e., the patients with substantially similar profiles with the subject patient but are taking the selected medication(s) for different indications), are monitored and the efficacy and safety of administering the selected medication(s) for their purposes, other than for Congestive Heart Failure, are evaluated and further considered in the present analysis.

Assuming that the above patients who are taking the medications for different indications are screened out, the remaining patients are further narrowed by using their profiles. That is, only those patients having substantially similar profiles to the profile of the subject patient are selected. As noted, a patient profile contains some or all pertinent information relating to a patient such as objective and subjective attributes. As noted, the patient profile database contains information relating to multiple patients' profiles including both subjective and objective attributes. The objective attribute may also include, as noted, one or more ICD-9 classification, and demographic information including the patient's race, age, sex, etc. The subjective attribute may include, as noted, a patient's pain level indicated by the patient and/or by a treating physician, and a satisfaction of using a selected medication by the patient. As noted, the subjective attributes may also include the result (e.g., rankings that measure the success rate) of using a selected medication to treat a particular patient having a particular medication condition or diagnosis. Either objective attributes or subjective attributes, or both, may be considered during the process of comparing patient profiles.

Thus, for instance, patients may be selected based on sex, race, or combination of both. Different weight may be assigned to different patient parameters depending on the particular medical indication being treated and on the particular patient being treated. Alternatively or additionally, patients may be selected based primarily on subjective attributes. Thus, only those patients having (or have had) similar pain levels as the subject patient, for instance, may be selected. Ultimately, the list of patients contains only those patients who meet the profile of the subject patient. Thereafter, PPOS 105 automatically analyzes the selected medications administered to the selected patients to treat Congestive Heart Failure. Depending on the analysis, PPOS 105 selects one or more medications that are most appropriate for the subject patient. In addition, other pertinent directions relating to the selected medication, such as the particular dosage, form (e.g., liquid, solid, or powder) and method of administration (e.g., intravenous, oral, or suppository) and length of treatment, are identified.

In accordance with one aspect of the present invention, the method of ultimately selecting one or more medications that are most appropriate for the subject patient can be approached from different perspectives. For instance, in the above example, PPOS 105 began the analysis by first looking up all medications that are used to treat Congestive Heart Failure, for example, in Physician Desk Reference. In another embodiment, PPOS 105 may begin the analysis by first looking up all patients in databases 120, 145 who match the profile of the subject patient, especially those who match the current indication of the subject patient. Thereafter, PPOS 105 analyzes the medications that are used or are being used by the selected patients to treat Congestive Heart Failure in. Ultimately, PPOS 105 selects one or more medications, and their dosages, method of administration and duration of treatment that are most appropriate for the subject patient.

In accordance with one aspect of the present invention, patients with a high match based on profile but who were not medicated may also be selected to see the effects of non-medication. Based on further analysis by a caregiver, an outcome could be that the caregiver prescribers no medication.

One of the features from above examples is that the method of the present invention is primarily based, not on how particular medication reacts in general, but on how the medication reacts to a particular patient having a particular indication. Using this feature, it is possible to predict with enhanced accuracy and reliability, for instance, how a particular medication (e.g., Hydralazine) would work in a particular patient having a particular medical indication. The patient parameter for the subject patient may indicate, among other things, that the patient is a 45-year-old, Caucasian male who is 5′ 10″ tall and weighs 185 lbs. Also, the patient suffers from headaches, rapid heartbeat, and joint pain. The patient profile stored in databases 120, 145 may further indicate the relevant medical history, such as his medical condition, other medications used or currently being used, an ICD-9 classification, etc. Note that while above examples illustrate certain embodiments of using PPOS 105, they are not meant to be inclusive of all embodiments. PPOS 105 can be used in many inventive ways to facilitate the methods of the present invention.

Referring again to FIG. 4B, in one embodiment, upon selecting the medication for the subject patient in step 440, the prescription can be processed automatically in step 460. For instance, using caregiver interface site 200, a caregiver can directly submit a request to fill the selected medication. The selected medication is then delivered directly to a destination chosen by the caregiver. The destination could be a hospice, pharmacy, or patient's home.

The medication selection and patient profile from above transaction is automatically updated to databases 120, 145, thereby updating and expanding the iterative database for each successive use. This is shown in step 480 of FIG. 4B. In this way, after selecting the medication and/or processing the selected medication, databases 120, 145 are updated to capture accurate and comprehensive information. The information updated, for instance, includes patient profile, medication files, medication inventory files, and other relevant files in databases 120, 145. Thus, the automatic update process facilitates more accurate functions of the present invention of using PPOS 105. For instance, as a particular medication is administered over time to a particular patient showing a particular indication, its result showing relevant and available (i.e., both objective and subjective) information including, for instance, medication's safety and efficacy along with subjective data such as patient's satisfaction towards the medication, is further discussed and studied by medical professional committee, and then may be used to update the treatment protocols of MUGs database.

As noted, in one embodiment, PPOS 105 uses, among other things, MUGs database to provide caregivers the ability to predict probable outcomes of using a given medication. Over time, MUGs database becomes more comprehensive and detailed with respect to selected medications. Accordingly, as the information stored in the MUGs database relating to selected medications and their safety and efficacy relating to multiple pools of patients becomes more detailed and complete over time, the utility of PPOS 105 will be further enhanced from the point of view of caregivers because they will be able to predict more rapidly, more accurately and with higher confidence the likely outcomes of using a particular medication to treat a particular patient having a particular indication.

FIG. 4C shows a flowchart illustrating a novel method, in accordance with another aspect of the present invention, to improve medication selection and dispensing for a subject patient by allowing caregivers to compare a profile of the patient to the profiles of other patients. As shown, in step 415, prescription data for a patient is received from a caregiver. The prescription data is submitted to network system 100 using any computing means such as personal computer 180 or Personal Digital Assistant 170. Using personal computer 180 or PDA 170, the caregiver may connect to main system 110 via network 10. Thereafter, the caregiver can access PPOS 105 via caregiver interface site 200.

Using information contained in the prescription data, in step 425, a patient profile of the subject patient if accessed from databases 120, 145. Databases 120, 145 comprise the patient database that includes, among others, patient profiles of each patients. In step 435, the patient profile of the subject patient is compared to patient profiles of other patients stored in databases 120, 145. Thereafter, in step 445, data for other patients having similar patient profiles—i.e., those having one or more subjective and/or objective attributes that are substantially similar to that of the subject patient—are retrieved.

As noted earlier, the objective attributes refer to those attributes that are readily observable or measurable, and that can be easily compared among all patients, whereas the subjective attributes refer to those attributes that may not be equally applicable to all patients. The objective attributes may include, for instance, the patient's medical files, demographic information (including the patient's race, age, sex, height, weight, etc.), ICD-9 primary code or term, ICD-9 secondary code or term, allergies, pharmacogenomic data, and clinical status. Pharmacogenomic data means, as used in this disclosure, genetic traits that expressly influence the transport, receptor action, and metabolism of medication. The subjective attributes may include, among others, a pain level and treatment procedure. The pain level indicates a level of pain expressed by the patient. The treatment procedure indicates all relevant procedures pertaining to using a particular medication. Thus, the treatment procedure for a particular patient may include, for instance, a dosage, a method of medication administration, and form of medication (e.g., liquid, gas, or solid). Using the patient profile, a success rate for that particular patient is determined by comparing the patient profile to other like patients having like indications. For example, the success rate may indicate how well a particular patient having a particular medical condition had reduced a pain level by taking a particular medication administered in a particular way. This information is used to selectively retrieve the data of other patients in step 445.

In step 455, all possible prescriptions for the subject patient are compared to drug choice protocols in MUGs database. As noted, MUGs database includes evidenced-based and clinician-based, clinical trials results of selected medications that serve as a guide for prescribing medication for certain medical indications. In one embodiment of the present invention, drugs used in treatment protocols under MUGs are covered by the per diem payment paid by the hospice, and drugs not part of the MUGs protocols must be paid by the hospice in addition to the per diem payment. In step 465, using the patient specific data retrieved in step 445 and treatment protocols acquired in step 455, appropriate medications for the subject patient are selected.

In step 475, the selected medications are prescribed for the subject patient. The prescription also includes all other pertinent information such as an appropriate dosage, a method of administrating the medication, and a form of medication. In step 485, the selected medication is dispensed to the subject patient.

It should be apparent from the foregoing description that there are multiple embodiments of the present invention for facilitating effective medication management. As noted earlier, the term “profiling” is used to describe the process of recording the medications a patient is taking (i.e., what has been prescribed and dispensed as well as the over-the-counter drugs), and the patient's demographic information, other objective attributes and subjective attributes relating to clinical status and outcomes. The utility of using PPOS 105 of the present invention should be apparent to those skilled in the art. As described further below, other embodiments of the present invention uses PPOS 105, especially caregiver interface site 200, and network system 100 to facilitate other online medication management functions, are within the scope and spirit of the present invention.

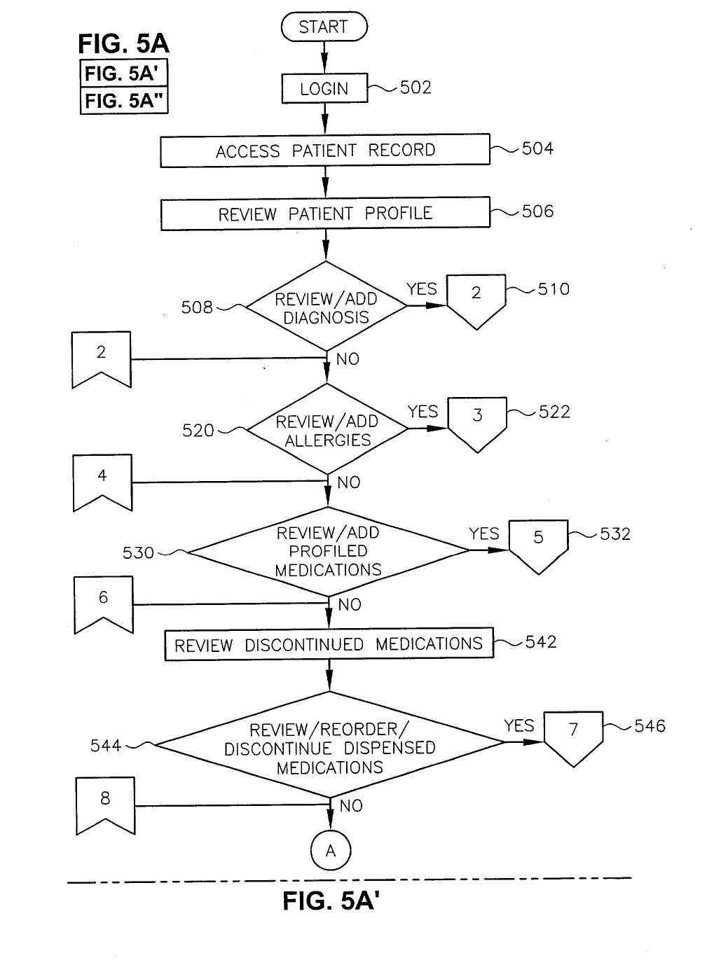

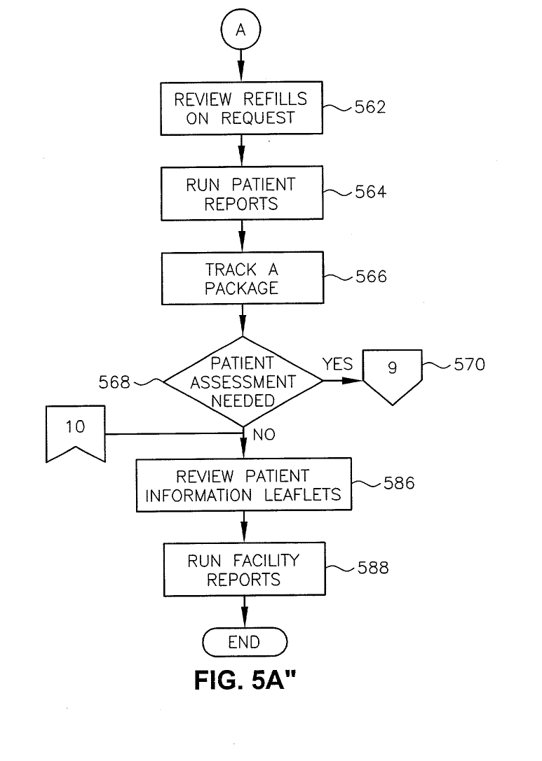

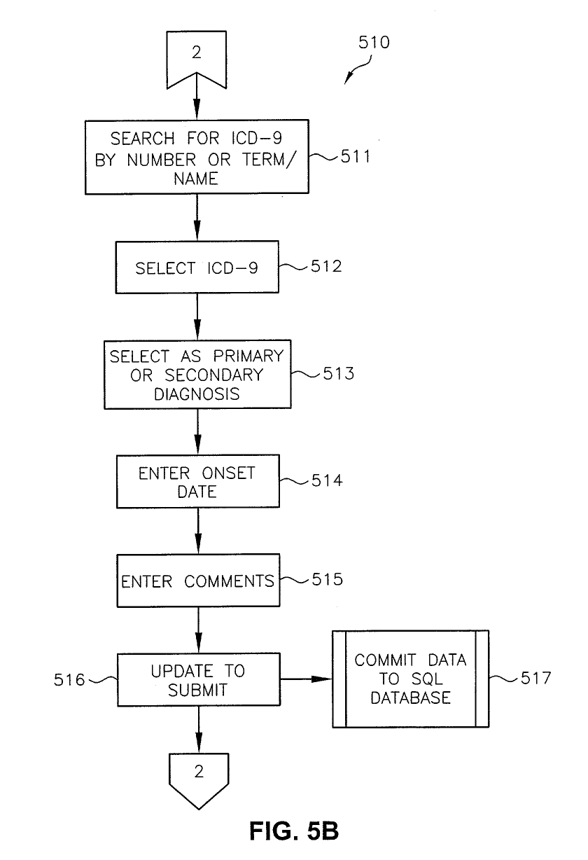

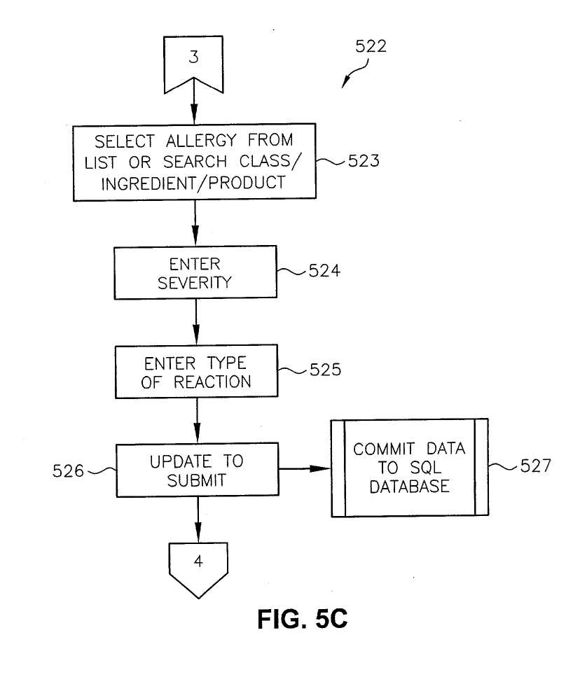

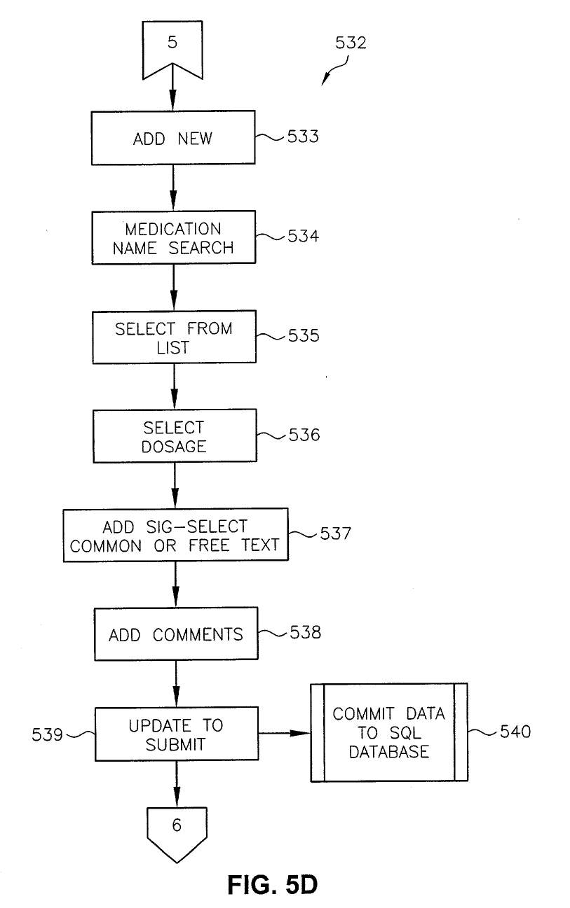

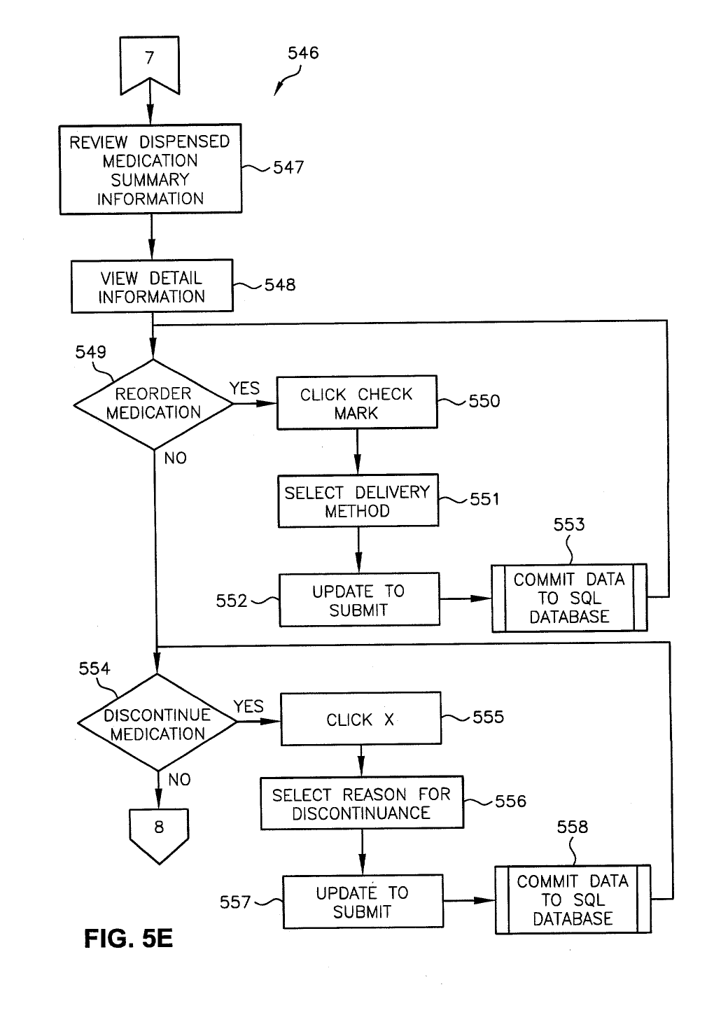

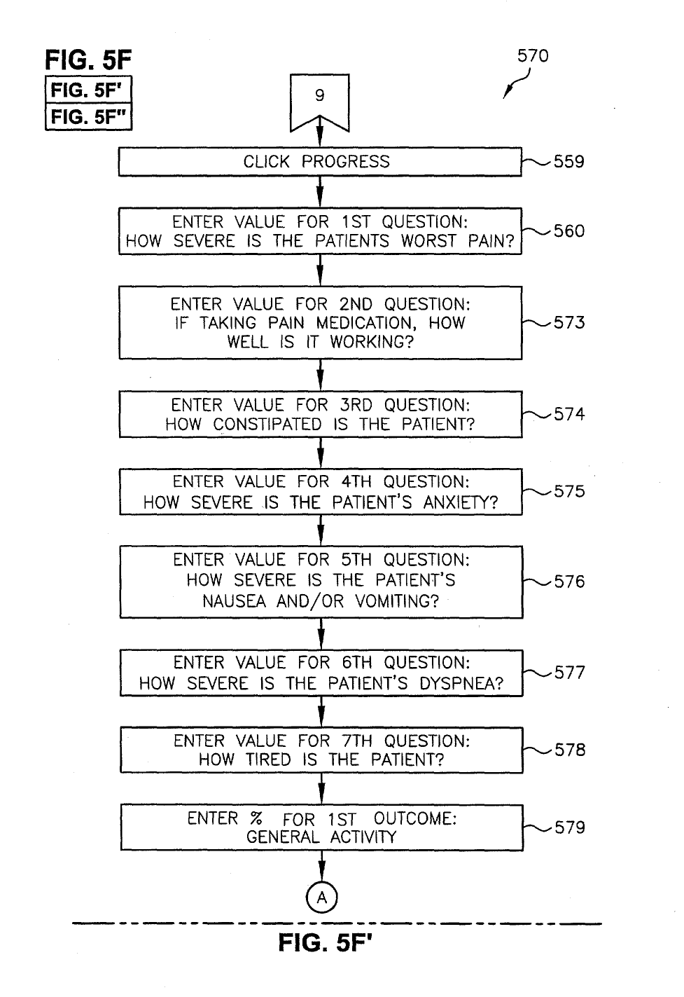

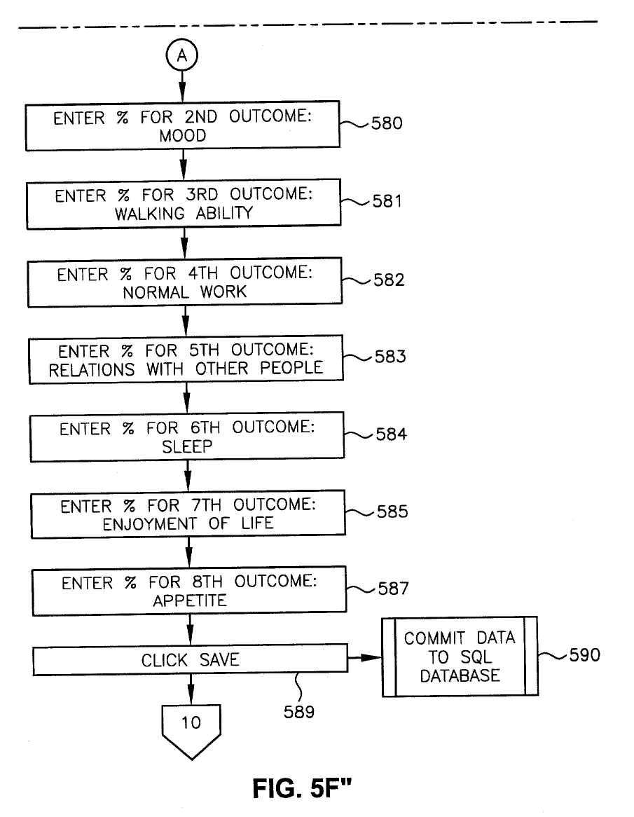

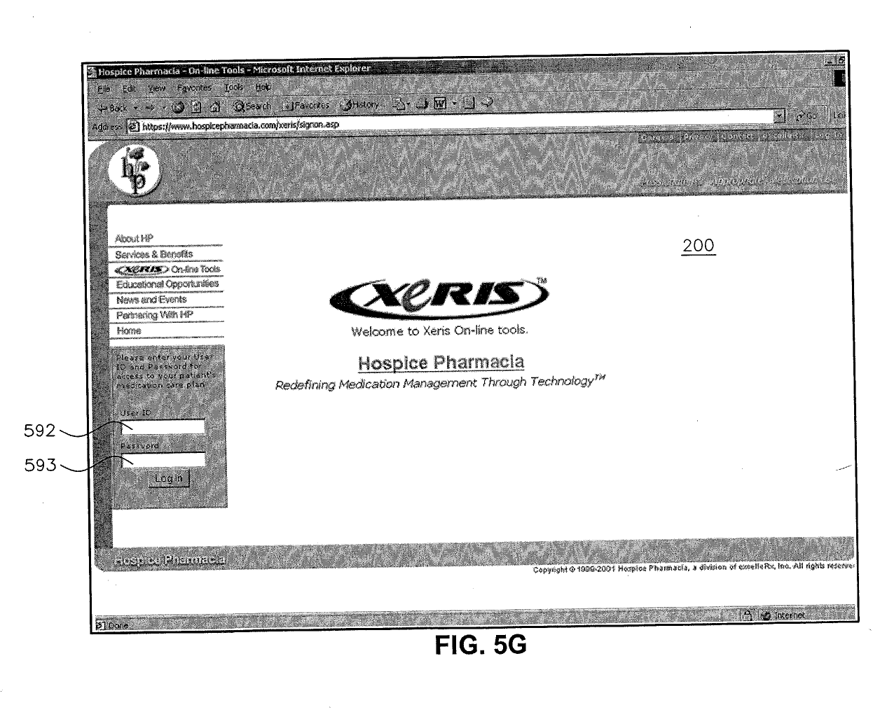

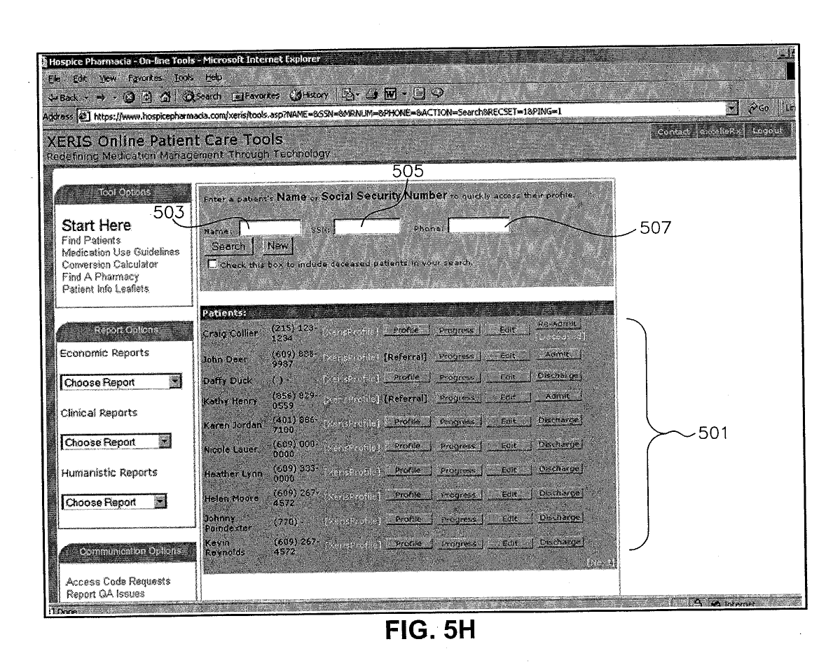

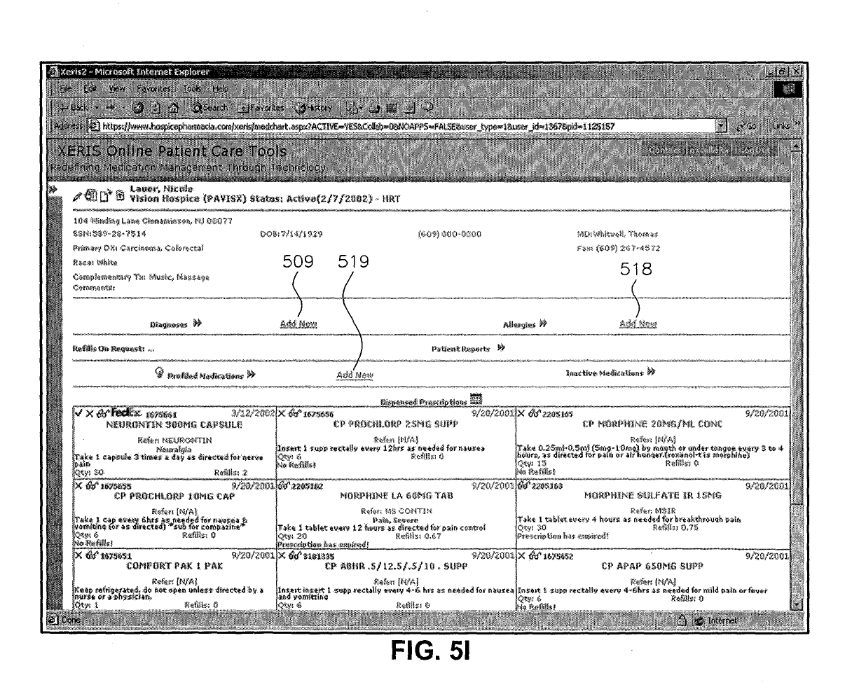

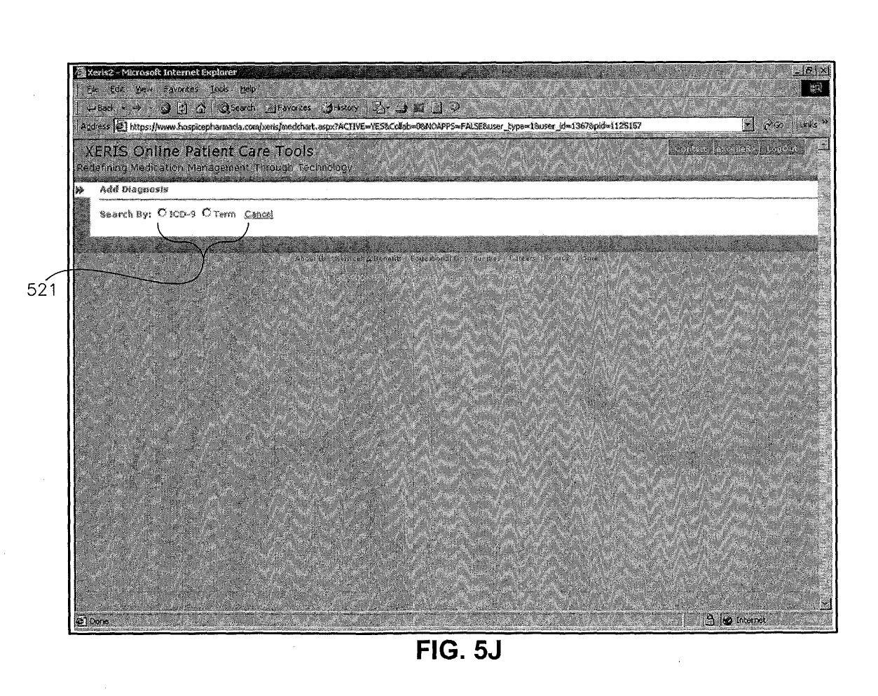

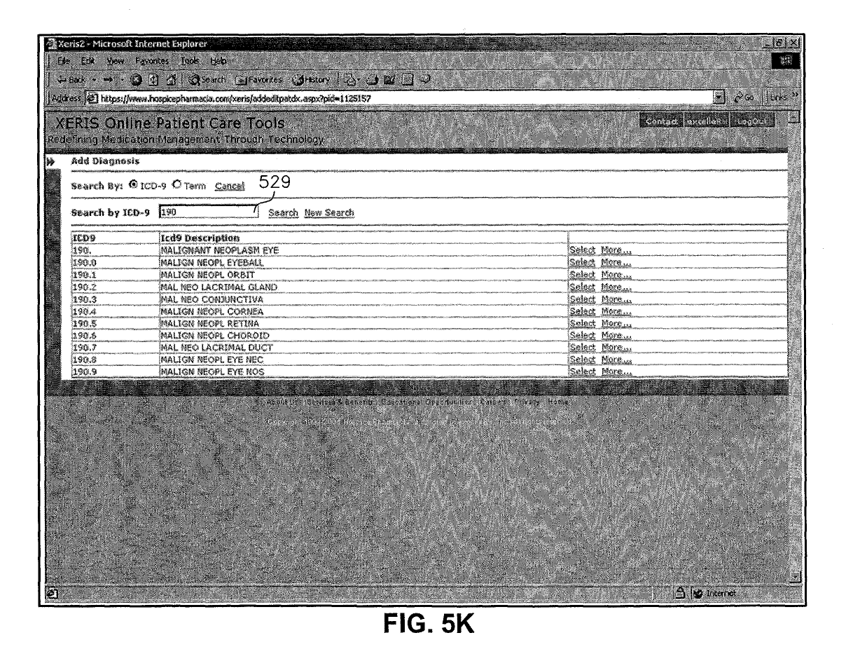

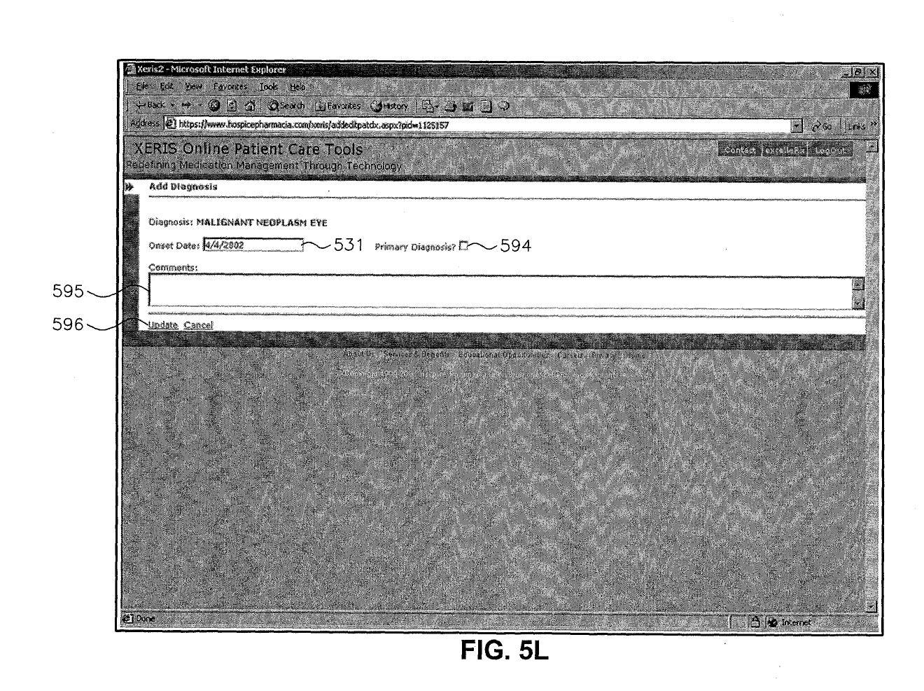

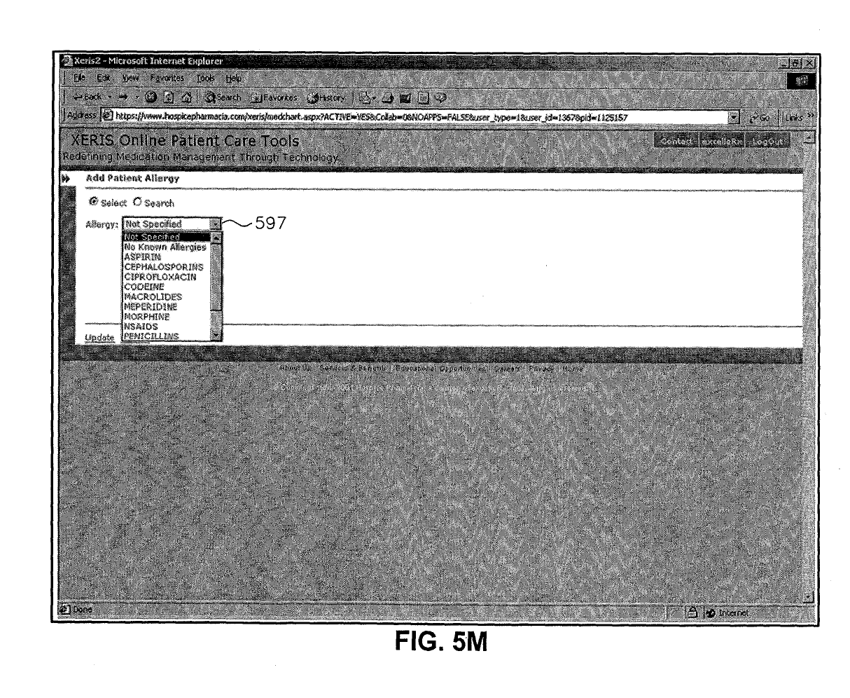

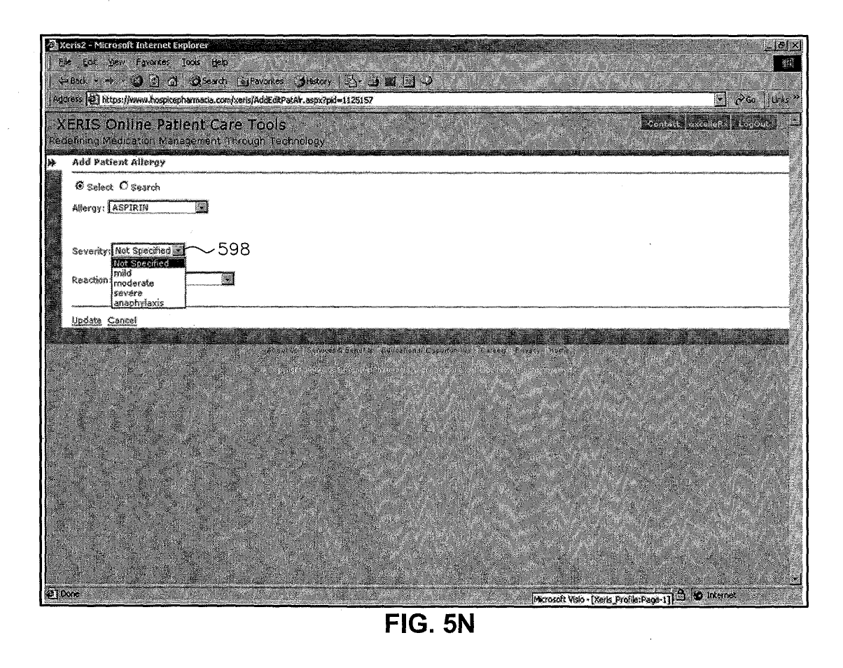

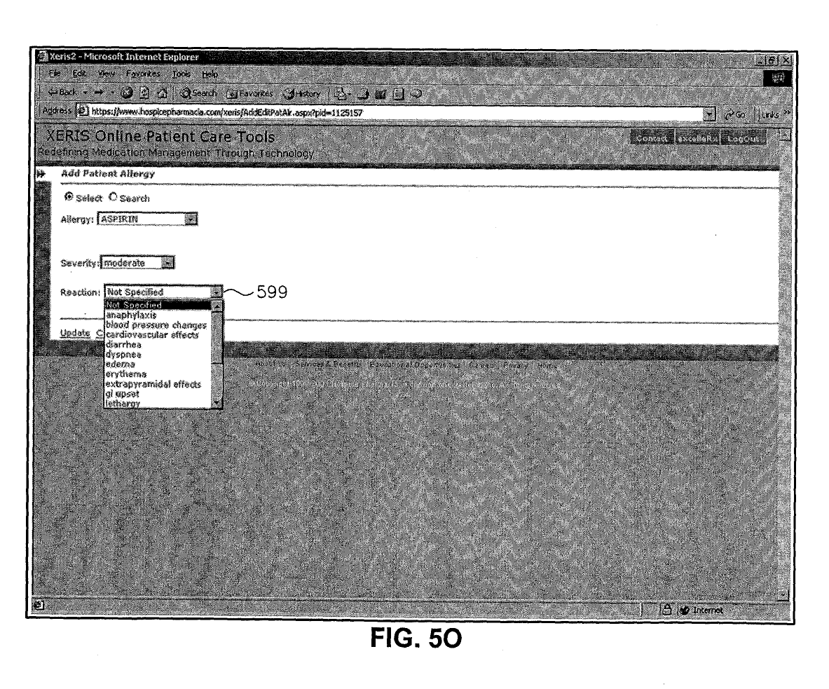

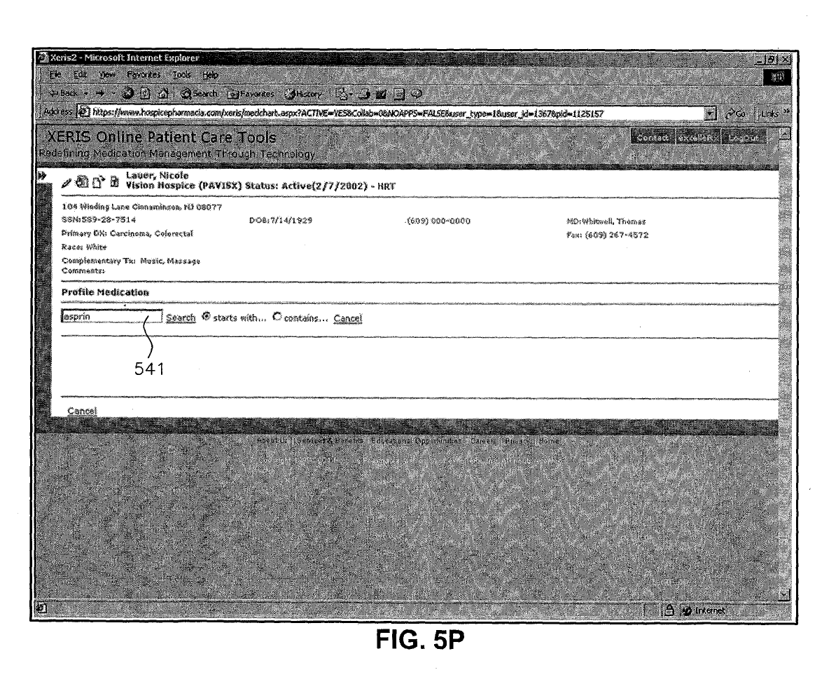

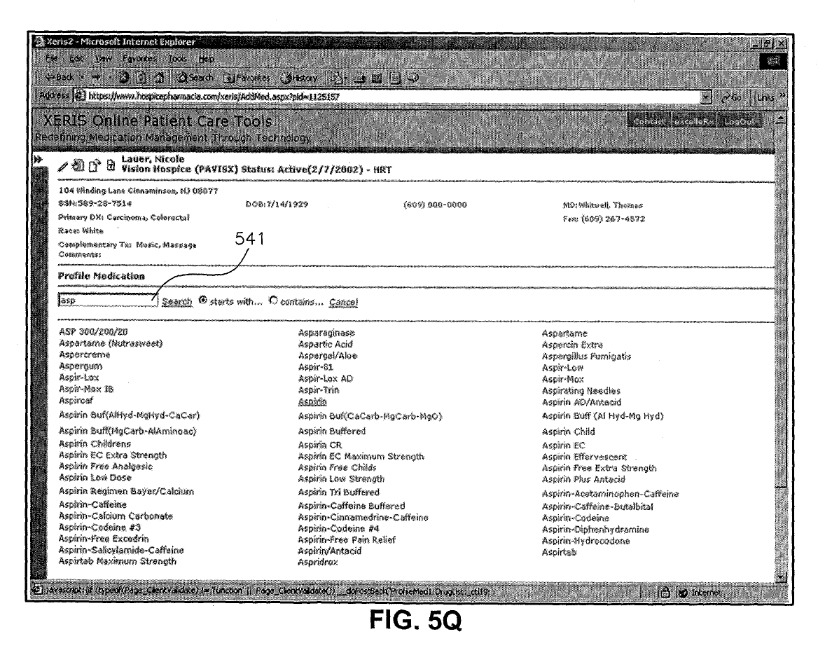

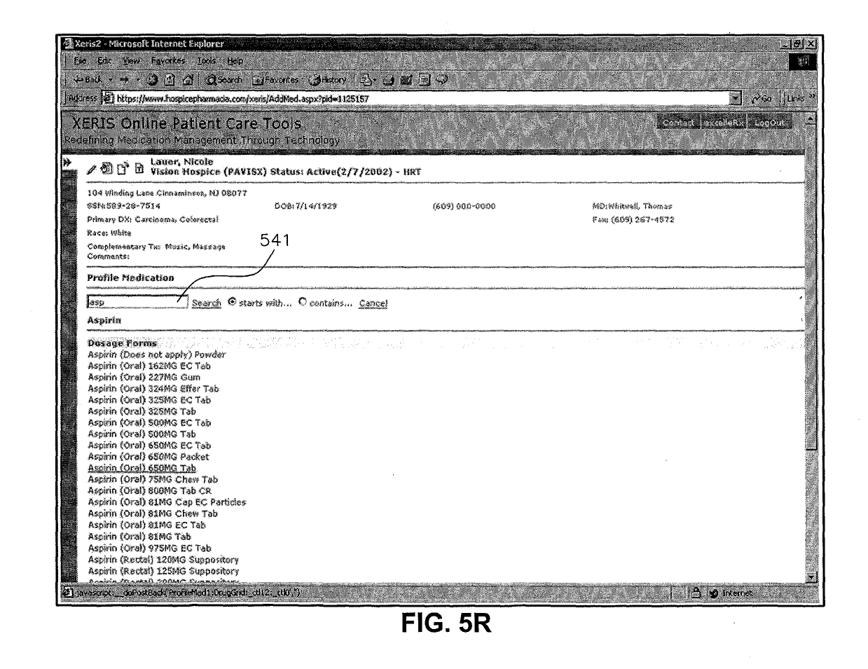

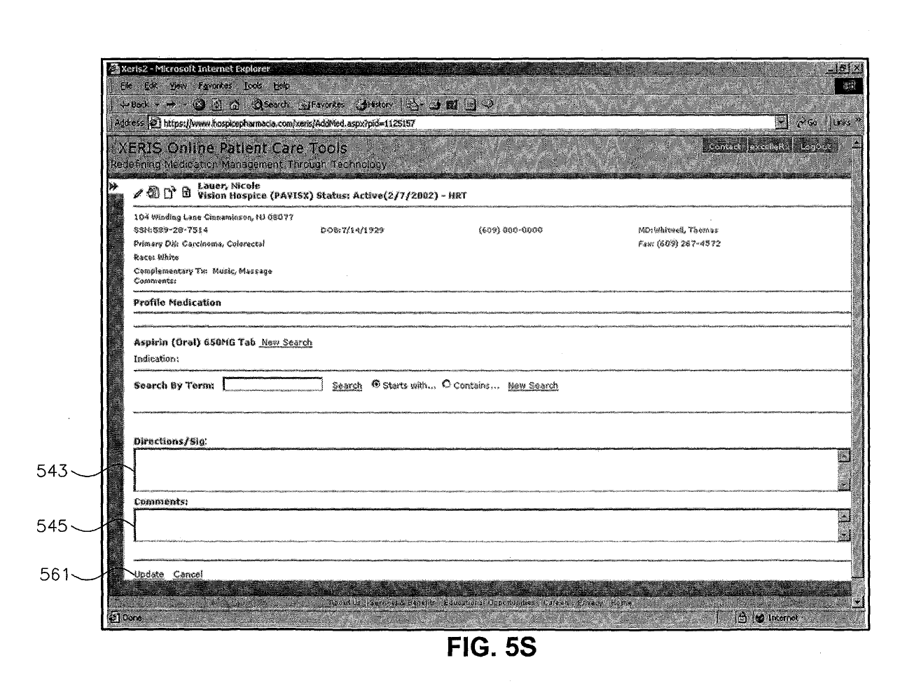

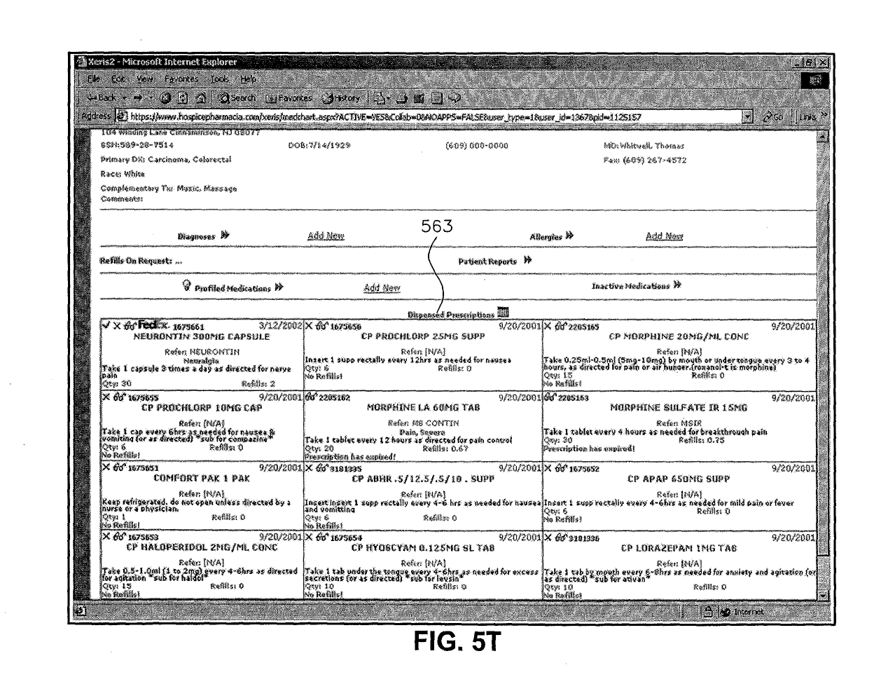

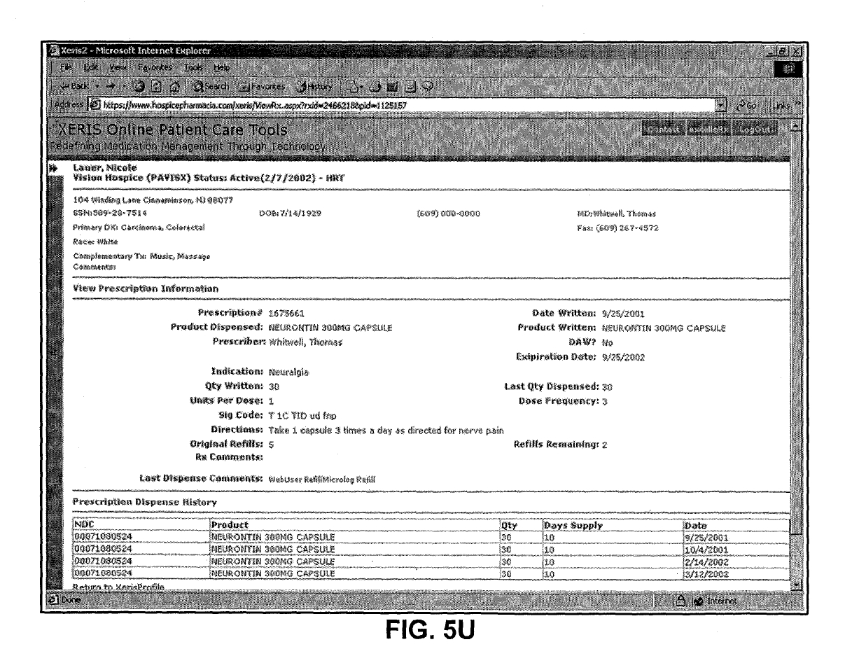

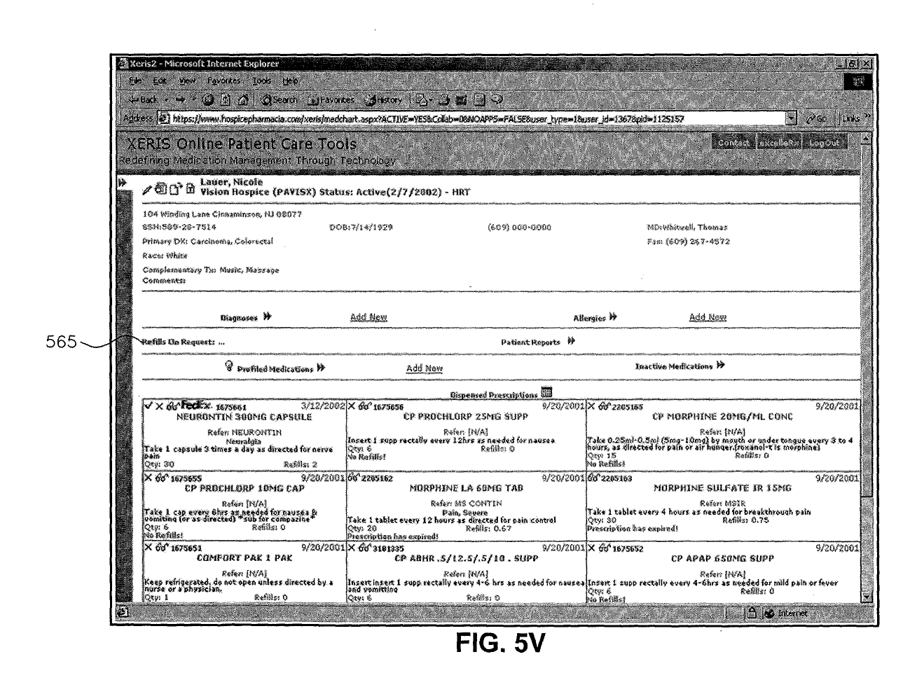

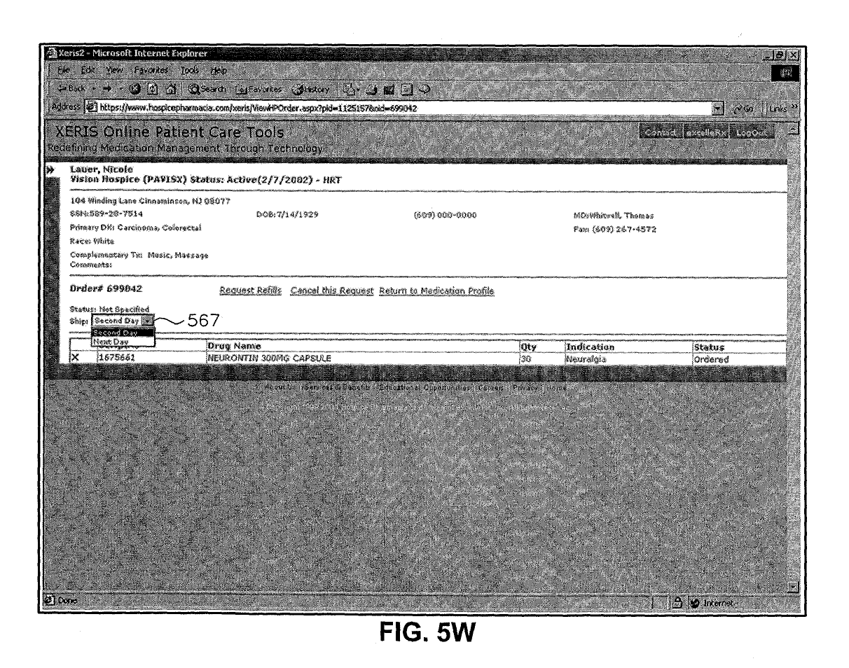

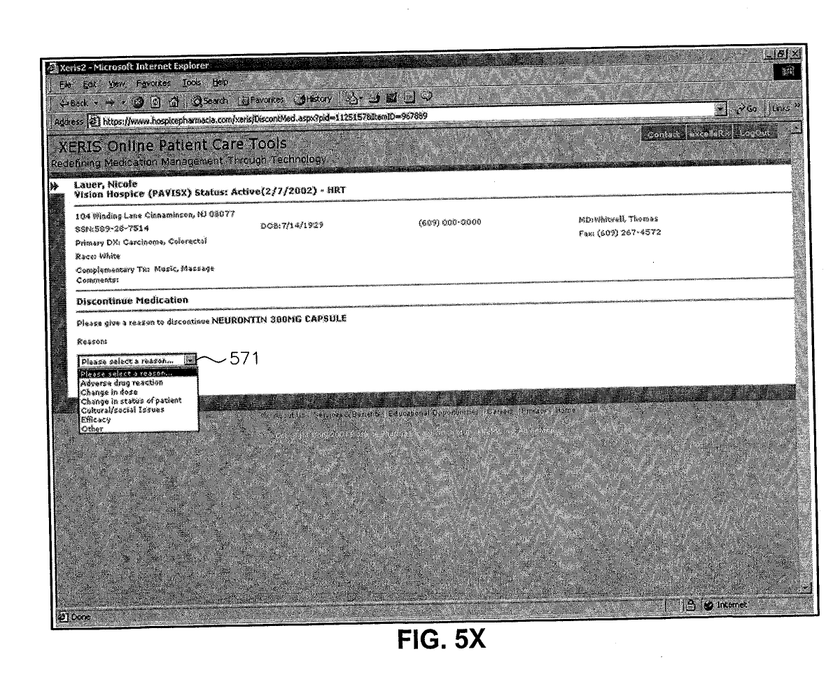

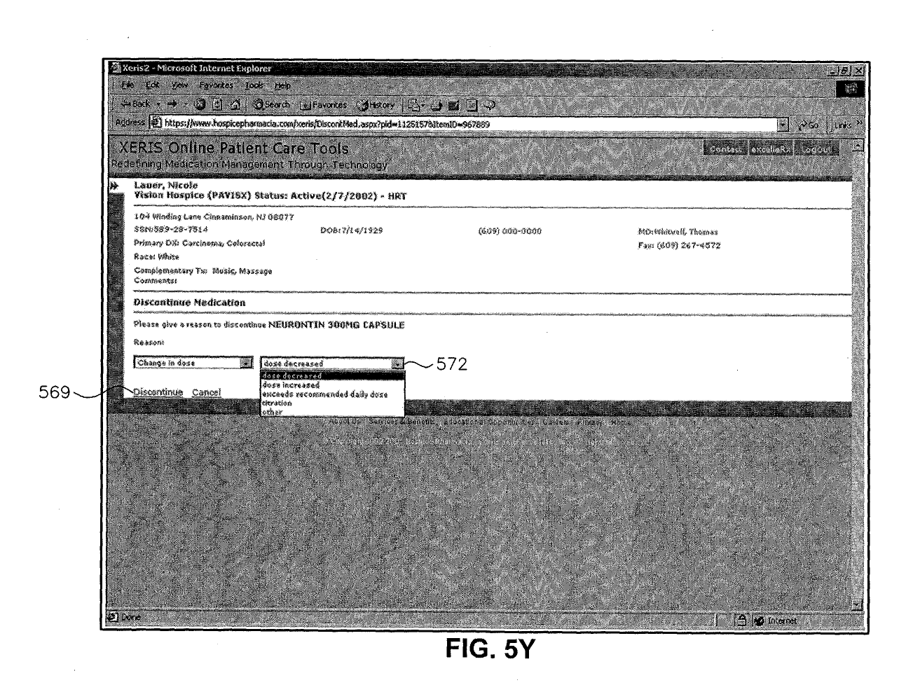

FIGS. 5A-5F show flowcharts illustrating one embodiment of the invention where certain caregivers, such as a physician or nurse in a hospice, use caregiver interface site 200 to directly profile medications using network system 100. Caregivers can use caregiver interface site 200 to view medications profiled, dispensed, discontinued, and also the refills requested in a “shopping cart” manner for all patients profiled. Further, caregiver interface site 200 can be used to update a medication profile, document more specific allergy information including severity, select reasons for discontinuation of therapy. Thus, complementary therapies and all other critical medical information can be documented using this web page

FIG. 5A is a flowchart illustrating an overview of this process according to one embodiment of the present invention. As shown, a caregiver logs in to network system 100 in step 502. The caregiver may log in using personal computer 180 or PDA 170 and gain access to caregiver interface site 200. In accordance with one aspect of the present invention, the caregiver may access all records of patients who are associated with the caregiver in step 504. In step 506, the caregiver may review any individual patient profile from the list of patients associated with the caregiver. In step 508, the caregiver may review and/or edit patient profile, as shown in step 510 and described further below, or just simply go to the next step. In step 520, the caregiver may review and add allergies to the patient profile, as shown in step 522 and described further below, or just simply go to the next step. In step 530, the caregiver may review and/or add profiled medications to the patient profile, as shown in step 532 and described further below, or just simply go to the next step.

Thereafter, the caregiver may review discontinued medications of the selected patient in step 542. That is, the patient profile contains not only the current medications that the patient is using, but also the list of discontinued medications, if any, for the patient. In step 544, the caregiver may review, reorder and/or discontinue dispensed medications, as shown in step 546 and described further below, or simply go to the next step. In step 562, the caregiver may review refills on request. Thereafter, the caregiver may run patient reports in step 564. A medication order can be tracked in step 566.

In step 568, the caregiver decides whether a patient assessment is needed, as shown in step 570 and described further below, or proceeds to the next step. In step 586, the patient information leaflets, which represent on-line documents showing in-depth information relating to specific medications or other medication related materials, can be reviewed. In step 588, facility reports can be generated.

In FIG. 5B, step 510 of FIG. 5A is illustrated in greater detail. As noted earlier, the caregiver may add diagnosis for any selected patient using, for example, one embodiment having web pages of caregiver interface site 200. In step 511, the caregiver may, prior to adding a diagnosis to the selected patient profile, use a search function to facilitate the diagnosis process. In accordance with one aspect of the present invention, the diagnosis process can be facilitated by searching for appropriate medical conditions using the International Classification of Disease (ICD) system. In one embodiment, the invention uses ICD-9 version. Using ICD-9, for instance, the caregiver can search for a medical condition. As noted earlier, the term “medical condition,” in accordance with one aspect of the present invention, refers to any cause, pathology, treatment or issue of an attack of disease, which serves as a guide or warning. The term medication indication is thus used interchangeably with the term disease, and therefore classified as a code in ICD-9 system. The caregiver may search for the medical condition in the ICD-9 system, which in turn facilitates the diagnosis process. As shown, the caregiver may choose to search the ICD-9 system by number or term name.

In step 512, the caregiver selects at least one of the medical conditions from the ICD-9 system. Using the chosen medical condition, in step 513, the caregiver may select the current diagnosis as either primary of secondary. In step 514, the caregiver enters the onset date of the diagnosis. Note that the caregiver can add any comments to the current diagnosis in step 515. Using this feature, the caregiver can explain otherwise unknown information. For instance, the comment can be used to explain why a particular diagnosis has been chosen as primary as opposed to secondary. In step 516, the caregiver updates the patient profile to reflect the recent transaction. Data representing the current transaction is then sent to the patient profile database, and to the SQL database, as shown in step 517.

In FIG. 5C, step 522 of FIG. 5A is illustrated in greater detail. One portion of the information contained in a patient profile is information relating to the patient's allergies. In accordance with one aspect of the present invention, this information can be added and/or edited to the patient profile by a caregiver directly. In step 523, while the caregiver is in the selected patient's profile, allergy information can be added and/or edited. It is possible to select allergy from a comprehensive list of allergies. Alternatively or additionally, it is possible to search for a specific allergy using allergy class, ingredient, and/or product. Upon selecting the allergy, its severity on the patient can be entered in step 524. In step 525, the type of reaction of the allergy can be entered. In step 526, the patient profile can then be updated to reflect the recent addition and/or edit relating to allergy. Data representing this update is sent also to SQL database, as shown in step 527.

In FIG. 5D, step 532 of FIG. 5A is illustrated in greater detail. As noted earlier, it is important to “profile” all medications that are associated with the patient—whether it is prescribed or purchased over-the-counter, whether it is current or discontinued, etc. In accordance with one aspect of the present invention, all medications relating to the patient can be profiled to the patient profile using one or more embodiments. In step 533, a new medication can be added. If exact information, such as a common name, is not known, the medication can be searched from a comprehensive list in step 534. Using the list, a specific medication can be selected in step 535. After the medication is selected, its dosage can be selected in step 536. Additional direction and/or SIG can be added in step 537. SIG can be chosen from common fields, such as “four times a day,” or could be specifically tailored to the patient profile using free text. Any additional information or comments can be added to the patient profile in step 538. In step 539, the patient profile can then be updated to reflect the recent addition and/or edit relating to the profiled medication. Data representing this update is sent to SQL database, as shown in step 540.

In FIG. 5E, step 546 of FIG. 5A is illustrated in greater detail. As noted earlier, the caregiver can monitor and record information relating to dispensed medications of the associated patients. In step 547, the caregiver reviews any dispensed medication summary information of a patient. The caregiver views detailed information relating to the dispensed medication summary in step 548. In step 549, the caregiver may choose to reorder a selected dispensed medication, as shown in step 550 by checking a mark of the selected medication. A delivery method is then selected in step 551. Thereafter, the patient profile is updated to reflect the reorder of the dispensed medication in step 552. Data representing this update is sent to SQL database, as shown in step 553.

Alternatively, as shown, the caregiver may choose to discontinue the selected dispensed medication in step 554. This process (i.e., discontinuing medication) can easily be done by selecting the dispensed medication in step 555. Any reason for discontinuing the medication is entered in step 556. Thereafter, the patient profile is updated to reflect the discontinuance of the dispensed medication in step 557. Data representing this update is sent to SQL database, as shown in step 558.

In FIG. 5F, step 570 of FIG. 5A is illustrated in greater detail. As noted, in accordance with one aspect of the present invention, the patient database includes patient profiles that represent both objective and subjective attributes. Furthermore, the subjective attributes represent otherwise immeasurable subjective factors such as a pain level experienced by a patient. In accordance with one aspect of the present invention, objective tests are generally used to quantify these subjective attributes. One example of the objective tests used in the embodiment, for instance, includes collecting a caregiver's assessment of patient evaluation systematically. While treating a patient, the caregiver systematically records its assessment or evaluation of the treatment on the patient systematically.

In step 559, the caregiver selects a progress option from caregiver interface site 200. The progress option directs the caregiver to one embodiment of caregiver interface site 200 that can be used to populate (i.e., record treatment information relating to a patient) the patient profile with the subjective attributes data relating to the treatment to the patient. Note that, in another embodiment of the present invention, the patient profile can be populated using the objective attribute data relating to the treatment to the patient.

In step 560, the caregiver enters the answer for the first question. As shown, the first question of the present embodiment relates to the severity of the patient's worst pain. In step 573, the caregiver enters the answer for the second question. As shown, the second question of the present embodiment relates to the type of a medication that the patient is taking and the effectiveness of the medication. In step 574, the caregiver enters the answer for the third question. As shown, the third question of the present embodiment relates to the constipation of the patient. In step 575, the caregiver enters the answer for the fourth question. As shown, the fourth question of the present embodiment relates to the patient's anxiety. In step 576, the caregiver enters the answer for the fifth question. As shown, the fifth question of the present embodiment relates to the severity of the patient's nausea and/or vomiting. In step 577, the caregiver enters the answer for the sixth question. As shown, the sixth question of the present embodiment relates to the severity of the patient's dyspnea. In step 578, the caregiver enters the answer for the seventh question. As shown, the seventh question of the present embodiment relates to the degree of the patient's tiredness. It should be apparent that the questions identified above are examples of one embodiment of the present invention, and as such, there are other embodiments having more or less number of questions and/or with different types of questions.

In step 579, the percentage of the first outcome is entered. As shown, the first outcome for the present embodiment relates to a general activity of the patient. In step 580, the percentage of the second outcome is entered. As shown, the second outcome for the present embodiment relates to the mood of the patient. In step 581, the percentage of the third outcome is entered. As shown, the third outcome for the present embodiment relates to the walking ability of the patient. In step 582, the percentage of the fourth outcome is entered. As shown, the fourth outcome for the present embodiment relates to a normal working ability of the patient. In step 583, the percentage of the fifth outcome is entered. As shown, the fifth outcome for the present embodiment deals with the patient's relations with other people. In step 584, the percentage of the sixth outcome is entered. As shown, the sixth outcome for the present embodiment relates to the patient's sleep. In step 585, the percentage of the seventh outcome is entered. As shown, the seventh outcome for the present embodiment relates to the enjoyment of life of the patient. In step 587, the percentage of the eighth outcome is entered. As shown, the eighth outcome for the present embodiment relates to the appetite of the patient.

It should be apparent that the outcomes identified above are examples of one embodiment of the present invention, and as such, there are other embodiments having more or less number of outcomes and/or with different types of outcomes. Using the subjective attribute data collected from the above process, the patient profile is populated. In step 589, the patient profile having all the answers and the percentage of outcomes are saved by clicking save on the present embodiment of caregiver interface site 200. In step 590, data representing this patient (i.e., patient profile having all the answers from the above process) is sent to SQL database.

FIGS. 5G-5Y depict exemplary web pages of the embodiment described above, showing caregiver interface site 200 that can be used by caregivers, such as a hospice, to directly profile medications using network system 100. As shown in FIG. 5G, a caregiver may log in to network system 100 from caregiver interface site 200. The embodiment of caregiver interface site 200 contains user ID box 592 and password box 593 that can be used to facilitate providing secure access only to authorized caregivers.

After logging into network system 100 using caregiver interface site 200, the caregiver can access the records of all patients associated with the caregiver. In FIG. 5H, a list of patients associated with the caregiver is shown in patient list area 501. Caregiver interface site 200, as shown in FIG. 5H, further include name box 503, Social Security Number (SSN) box 505, and phone box 507 that facilitate searching of patients. The caregiver can find a patient, for example, by entering appropriate terms in the boxes. Note that the names of patients shown in patient list area 501 appear in hyperlinks, which can be “drilled down” (i.e., retrieving more information that may be placed in the same page or other entirely different pages by simply clicking on the hyperlinks) further to view information relating to a particular patient by clicking the patient's name shown from patient list area 501. A patient profile of the selected patient is shown in FIG. 5I. As shown, a new diagnosis or new allergy can be added by clicking on add new diagnosis link 509 or add new allergy link 518, respectively. New medications can also be added by using add new profiled medications link 519. Also, the caregiver can search for an appropriate diagnosis using search function. As shown in FIG. 5J, the caregiver can search for a diagnosis by selecting either ICD-9 code list or term names used in ICD-9 classification. This option can be selected by checking appropriate boxes in search diagnosis area 521.

FIG. 5K shows a list of ICD-9 codes having their descriptive terms placed next to the codes. Note that the search for the list shown is done by using ICD-9 code list. In this example, code 190 was entered in search term box 529. For example, in FIG. 5K, the search result shows all ICD-9 codes containing code 190 (e.g., Malignant Neoplasm Eye) is displayed as a result. Note that each code shown in the list is in hyperlinks, which can be selected by clicking on any particular code link. Once a particular ICD-9 code is selected and added to the patient profile, more information that defines the code specifically can be added. As shown in FIG. 5L, for instance, by using primary diagnosis box 594, the caregiver can designate whether the diagnosis is primary or secondary. The embodiment of caregiver interface site 200 shown in FIG. 5L further includes onset date box 531 and diagnosis comments box 595 that facilitate capturing of more detailed information relating to the selected diagnosis. Once the diagnosis is profiled, it can be updated using update link 596.

As noted earlier, in accordance with one aspect of the present invention, a patient profile also includes information relating to patient's allergy. FIG. 5M shows one embodiment of caregiver interface site 200 illustrating allergy drop down menu 597, reached through add new allergy link 518, that can be used to select a type of allergy. FIG. 5N shows another embodiment of caregiver interface site 200 illustrating severity drop down menu 598 that can be used to select a severity for the chosen allergy. FIG. 5O shows yet another embodiment of caregiver interface site 200 illustrating allergy reaction drop down menu 599 that can be used to select a reaction for the chosen allergy. All of the allergy information associated with the selected patient can be profiled in the patient profile.

Further, a patient profile also includes information relating to medication. The medication information, in accordance with one aspect of the present invention, is “profiled” to the patient's profile. FIG. 5P shows one embodiment of caregiver interface site 200 that can be used to profile a medication, to check whether the patient is currently taking a particular medication, and to add the medication to the patient profile. As shown, a medication to be profiled can be searched by entering a letter(s) in search medication box 541. FIG. 5Q shows a list of medications matching the search criteria. Note that names of the medications displayed in FIG. 5Q are shown in hyperlinks. Thus, by clicking on the name itself, the medication can be selected for profiling. While selecting the medication, appropriate dosage, form and method of administration for the medication can be selected at the same time. As shown in FIG. 5R, the caregiver can select, for example, Aspirin (Oral) Tablet having 650 MG by clicking on the name. Also, the selected medication can be profiled further by adding all relevant information associated to the medication. For instance, as shown in FIG. 5S, further information relating to directions for use of the medication to be written on a prescription label can be included in medication direction/SIG box 543 and medication comments box 545. After the medication has been profiled, it can be updated to databases 120 by clicking medication update link 561.

As shown in FIG. 5T, caregiver interface site 200 can also be used to review dispensed medication information. This can be done by clicking dispensed prescriptions link 563. As shown, all dispensed medications relating to the selected patient are then displayed. Note that the names of the dispensed medications are displayed in hyperlinks, which can be selected by clicking on the name to provide comprehensive information regarding the medication. FIG. 5U shows a profile of the selected medication. As shown, the profile contains in-depth detail information relating to the medication. As shown in FIG. 5V, any of the medication displayed can be selected and refilled or discontinued, if desired, by clicking on medication refill request link 565. As shown in FIG. 5W, a desired delivery method and timing for delivery for the medication can be selected by using medication delivery drop down menu 567. In one embodiment of the present invention, the payor for the medication will receive a discount for selecting a delivery option that is delayed for a period of time (e.g., 2 days vs. 1 day or 1 day vs. same day).

Referring again to FIG. 5V, any medication shown on the list can be discontinued by the caregiver. As shown in FIGS. 5X and 5Y, after selecting a medication, the caregiver can discontinue the medication by clicking on medication discontinue link 569. As shown, specific reasons for discontinuing medication can be profiled by using medication discontinue drop down menu 571 and secondary discontinue drop down menu 572.

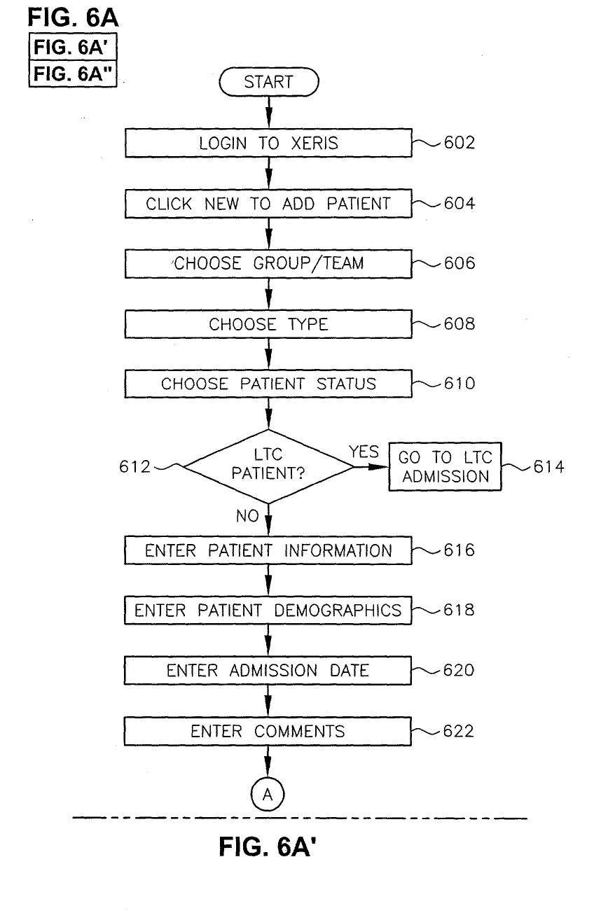

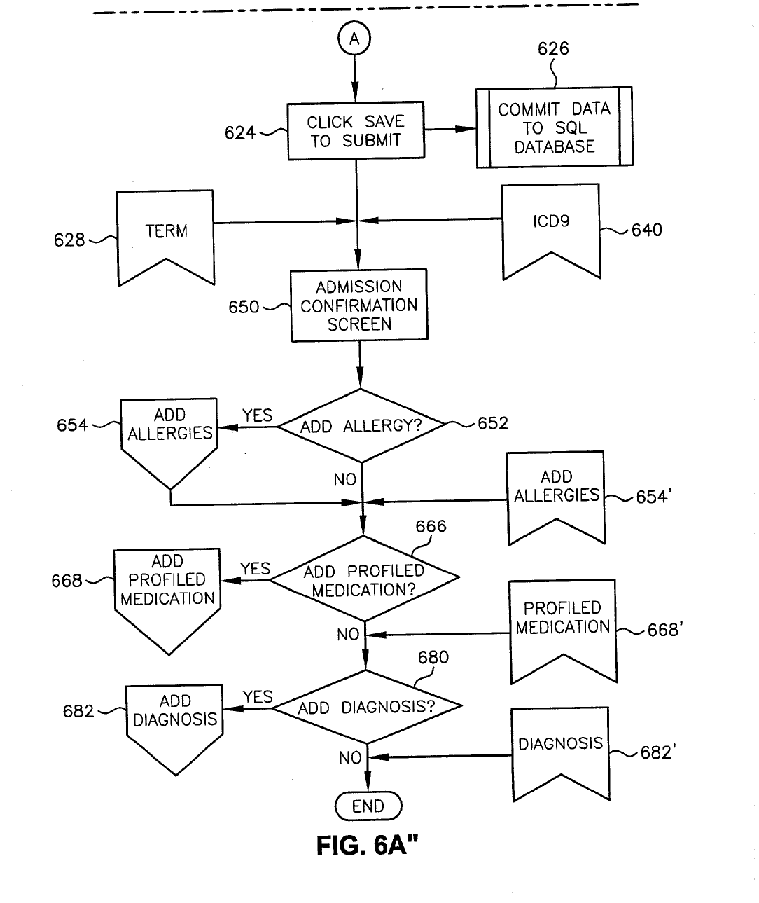

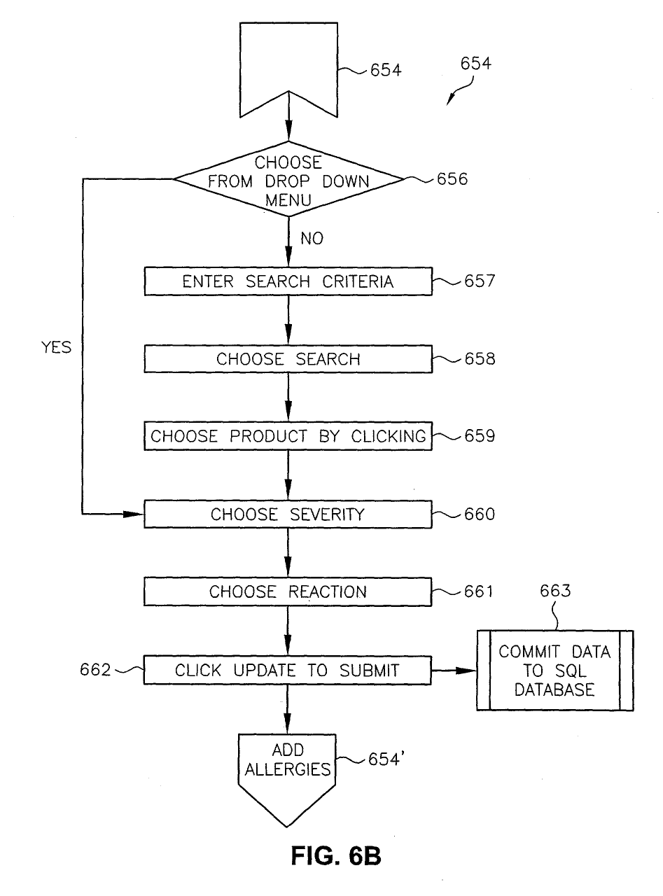

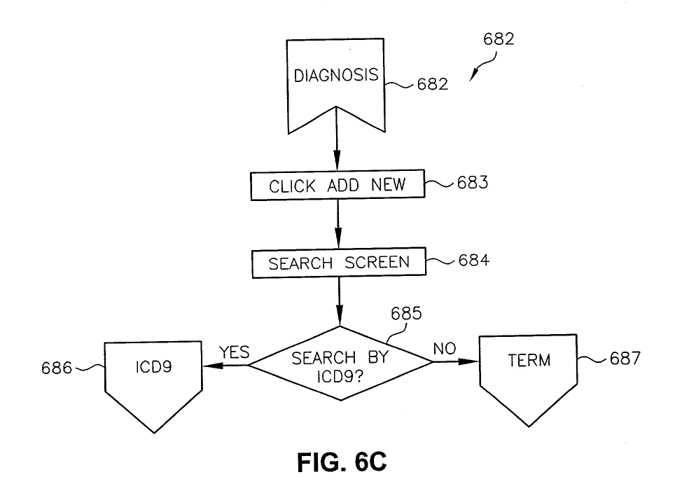

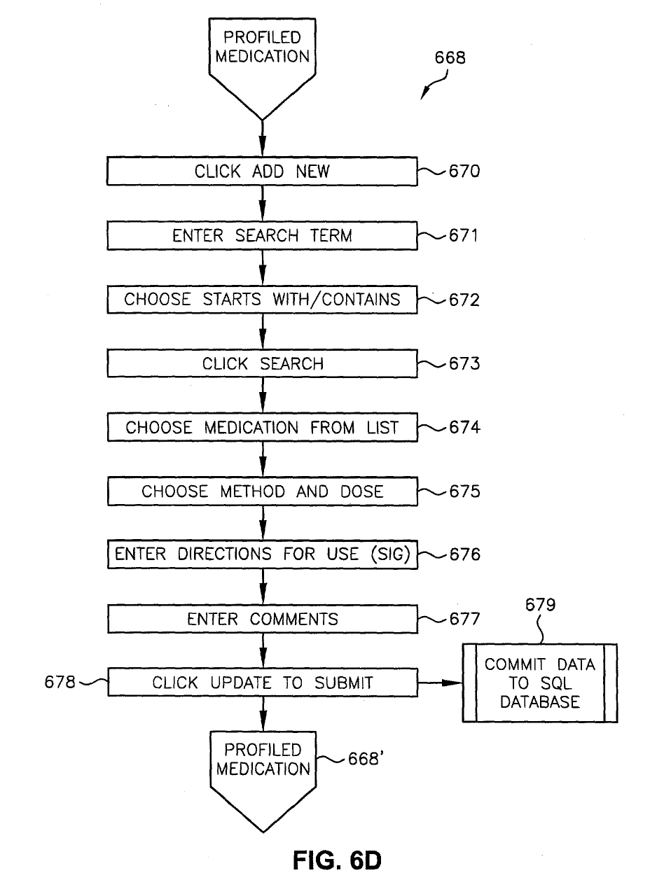

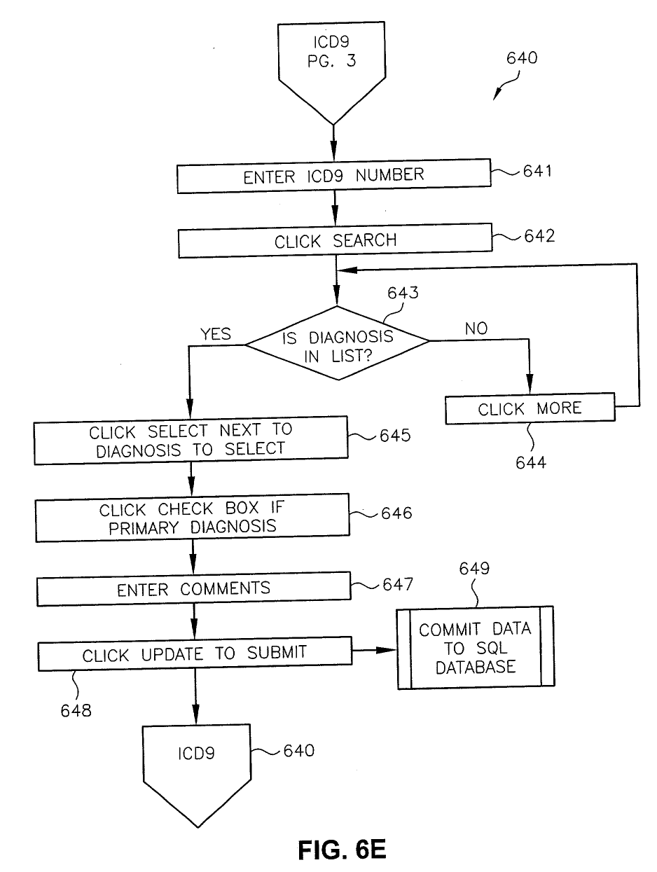

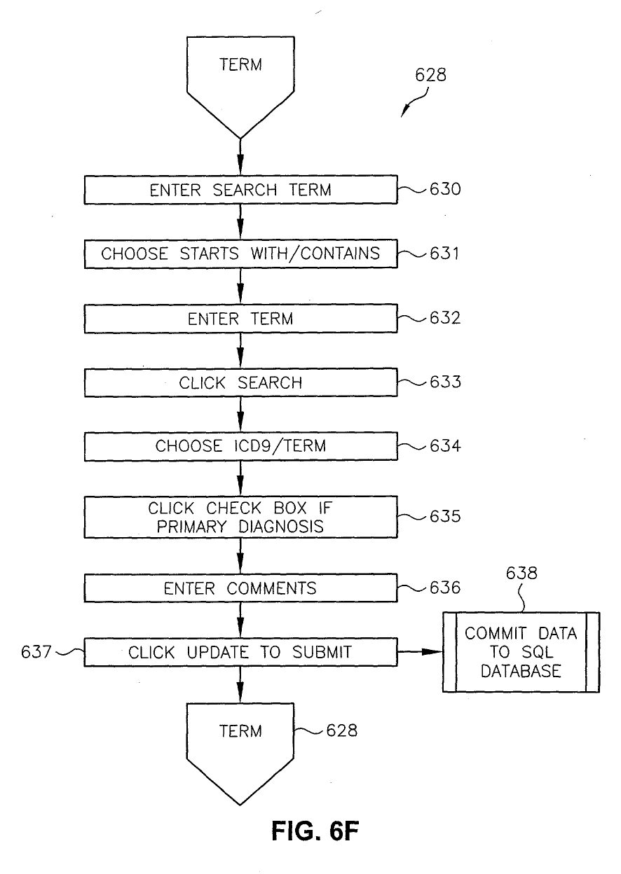

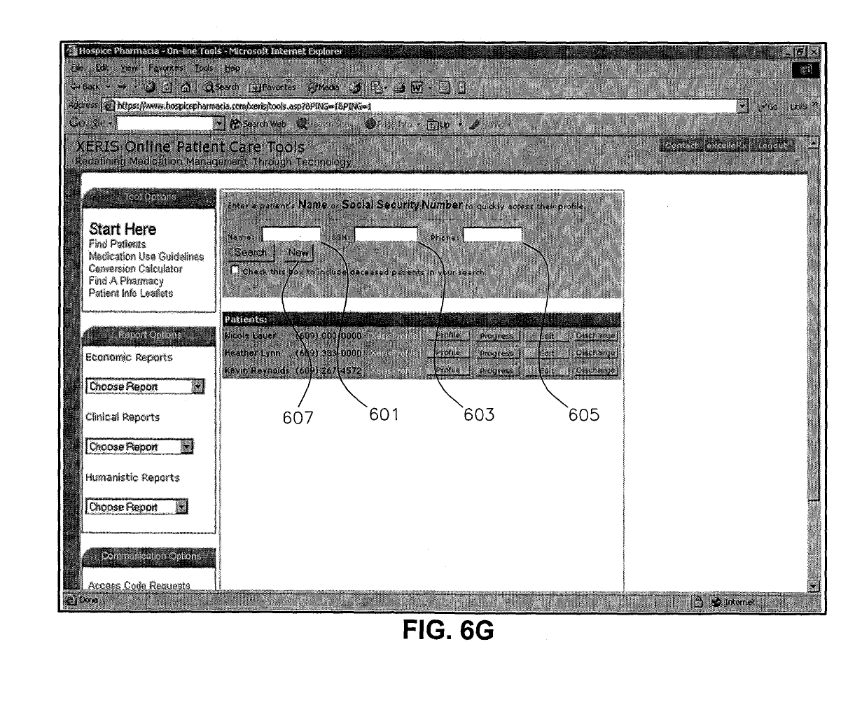

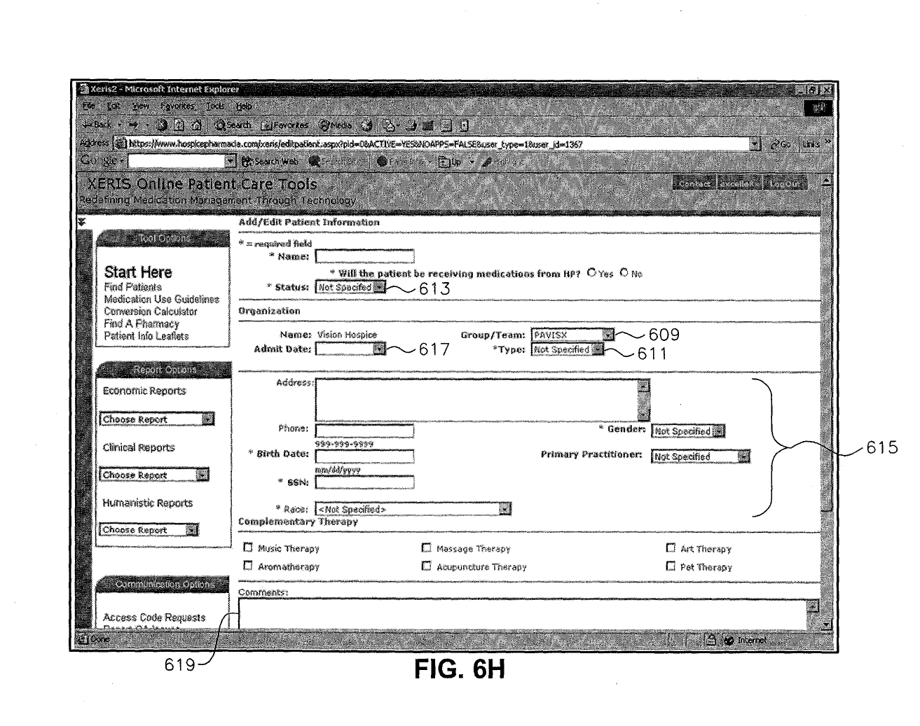

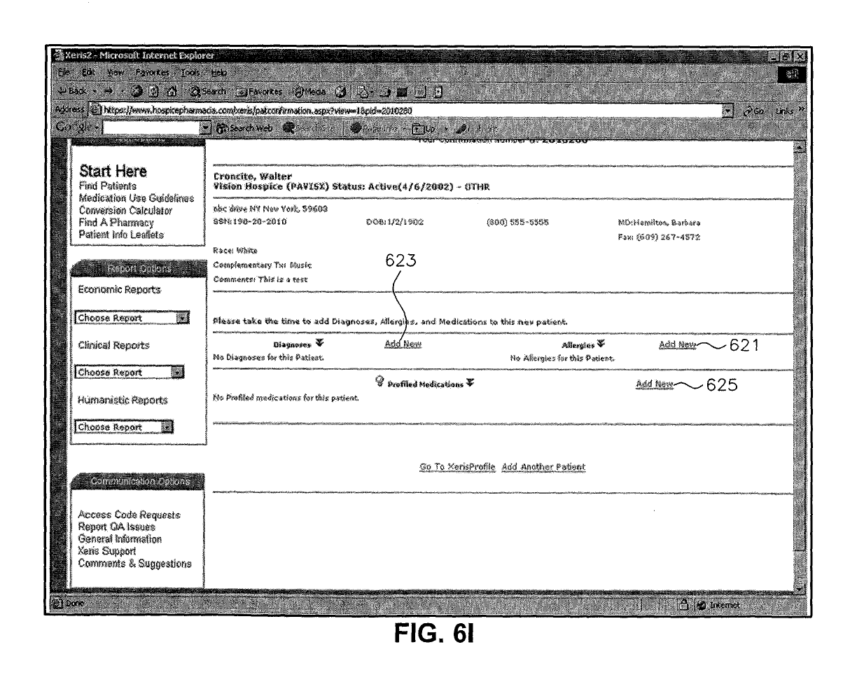

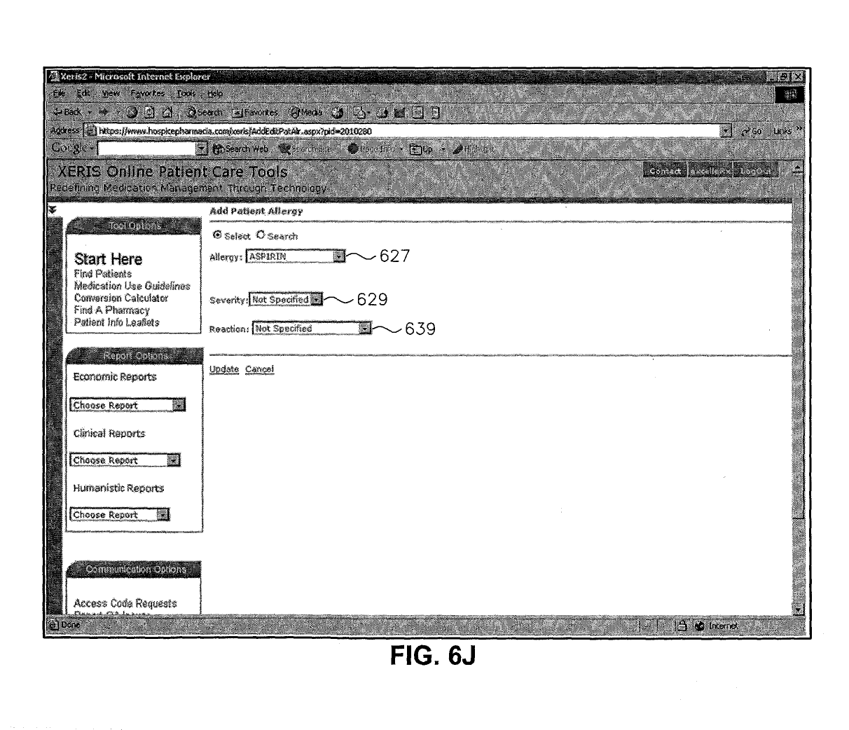

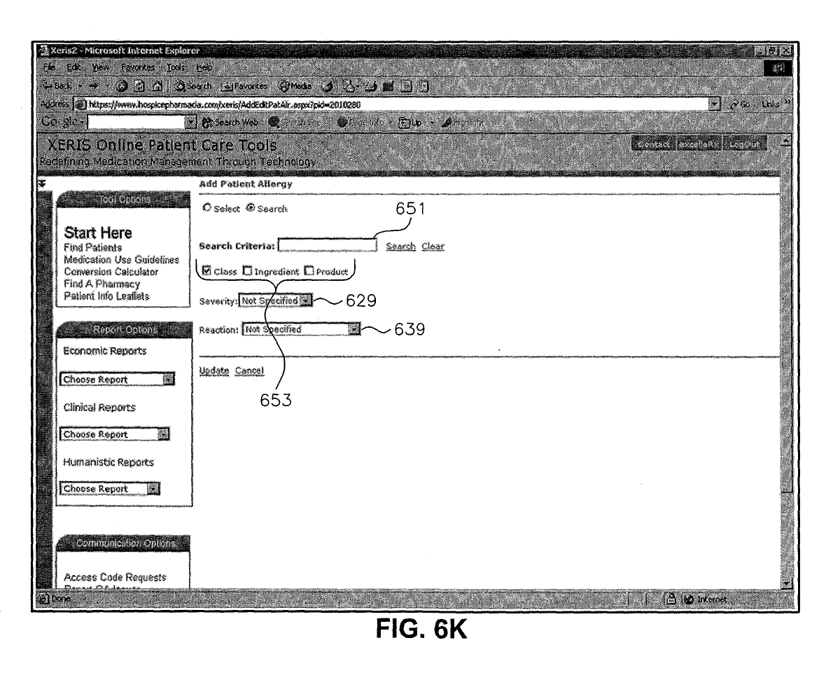

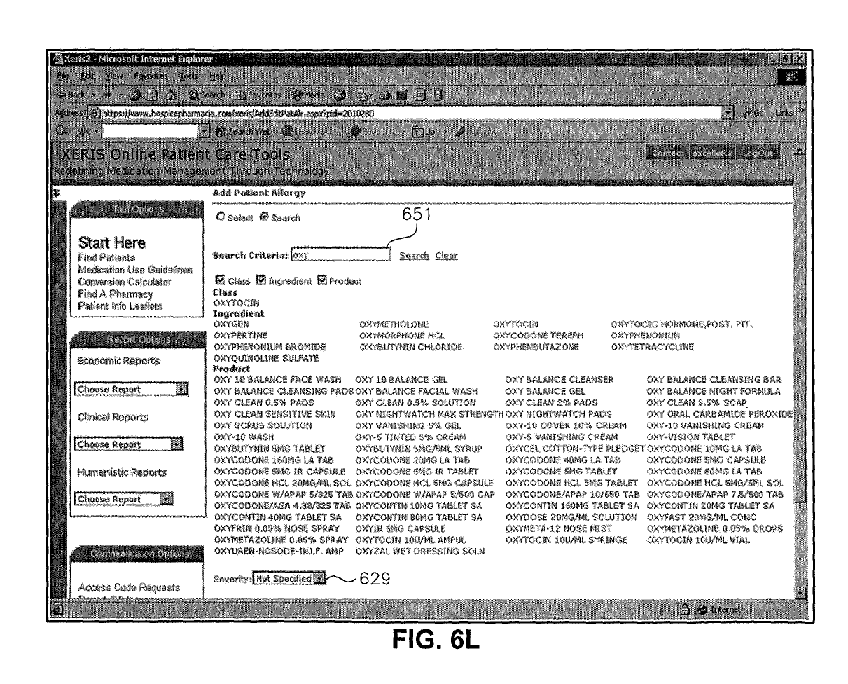

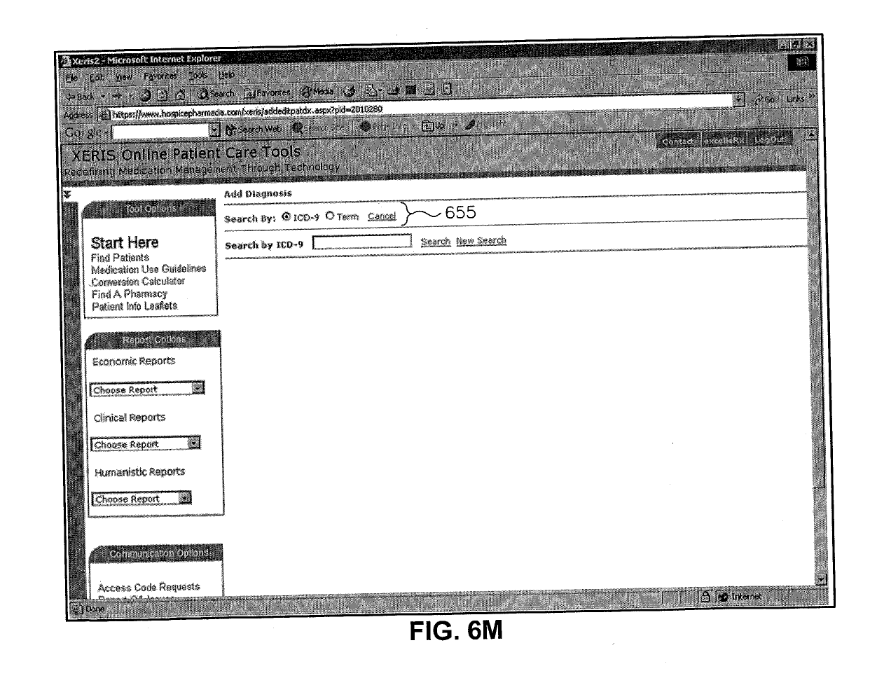

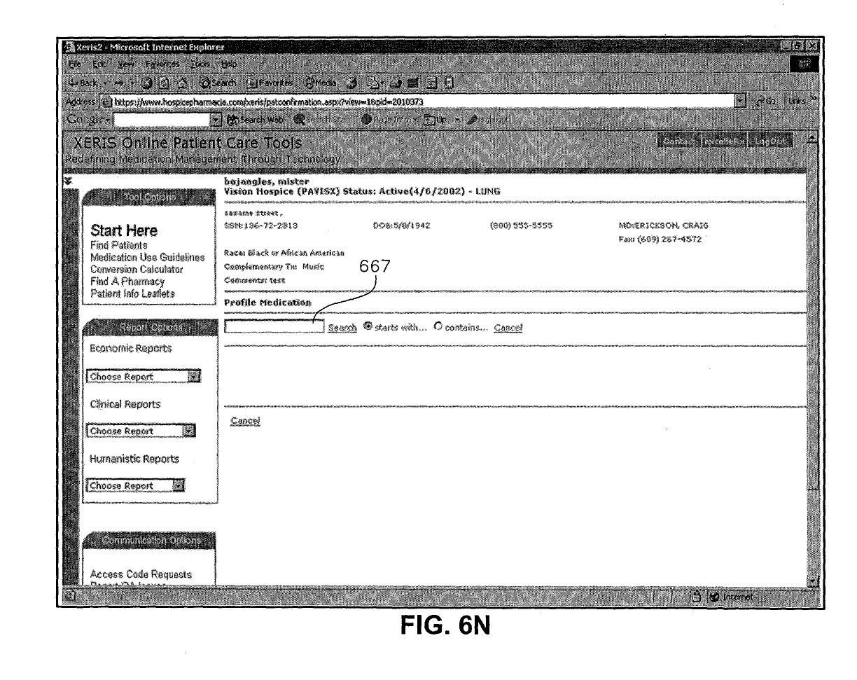

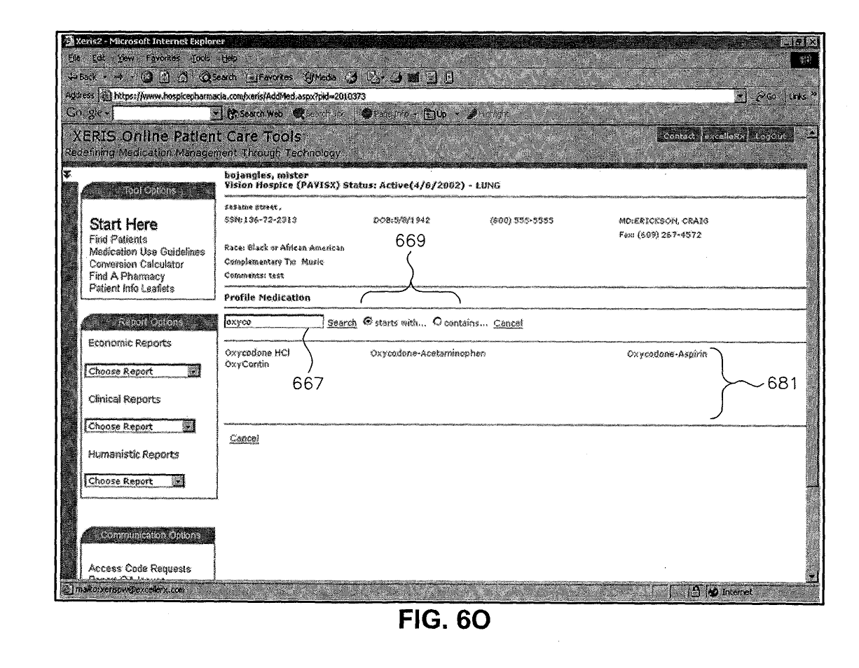

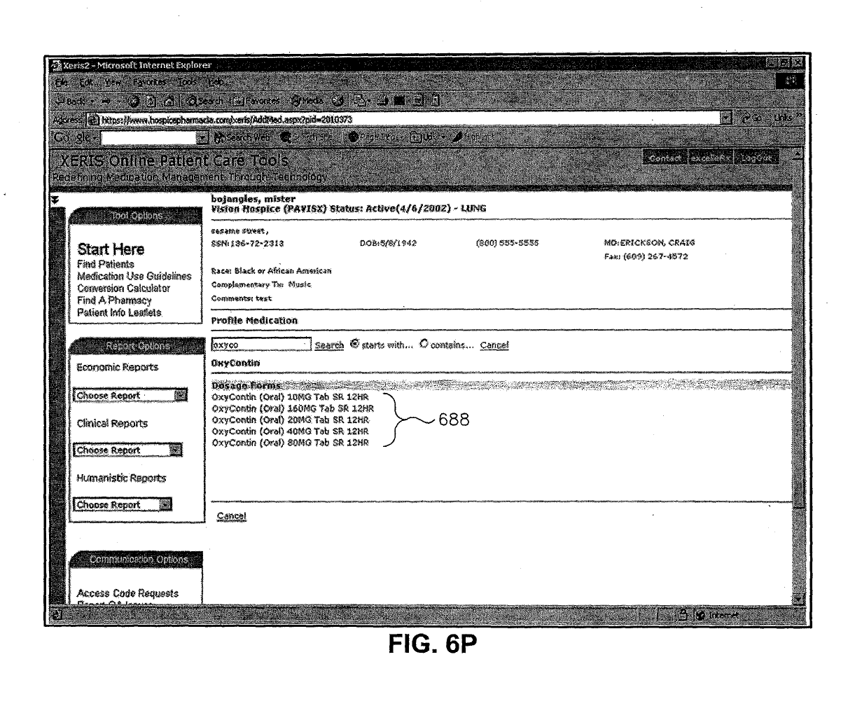

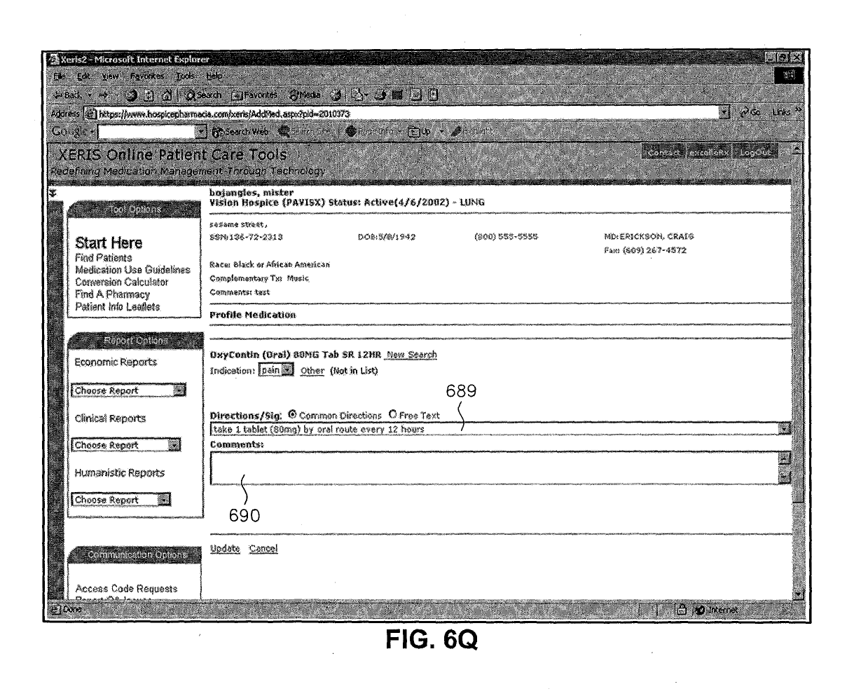

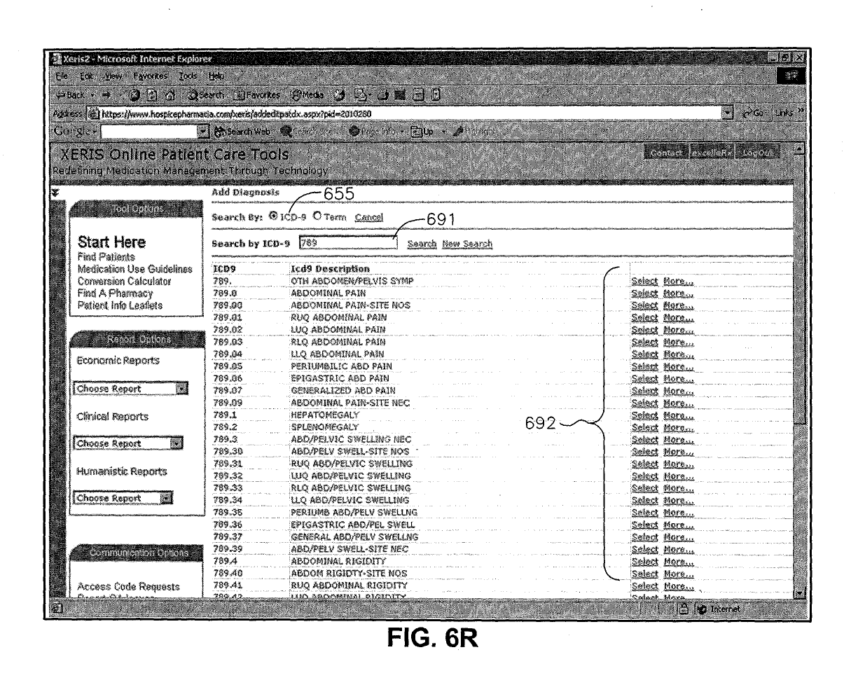

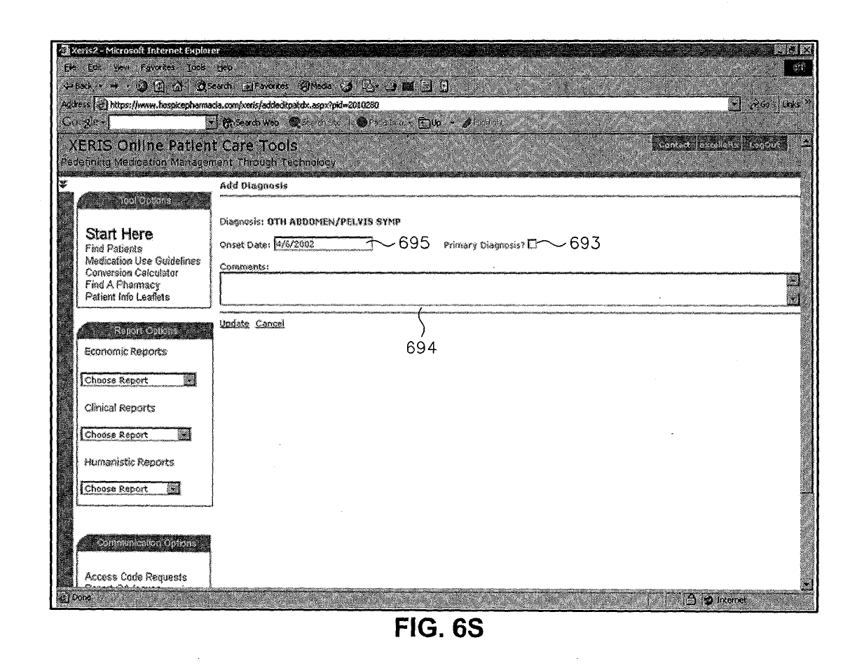

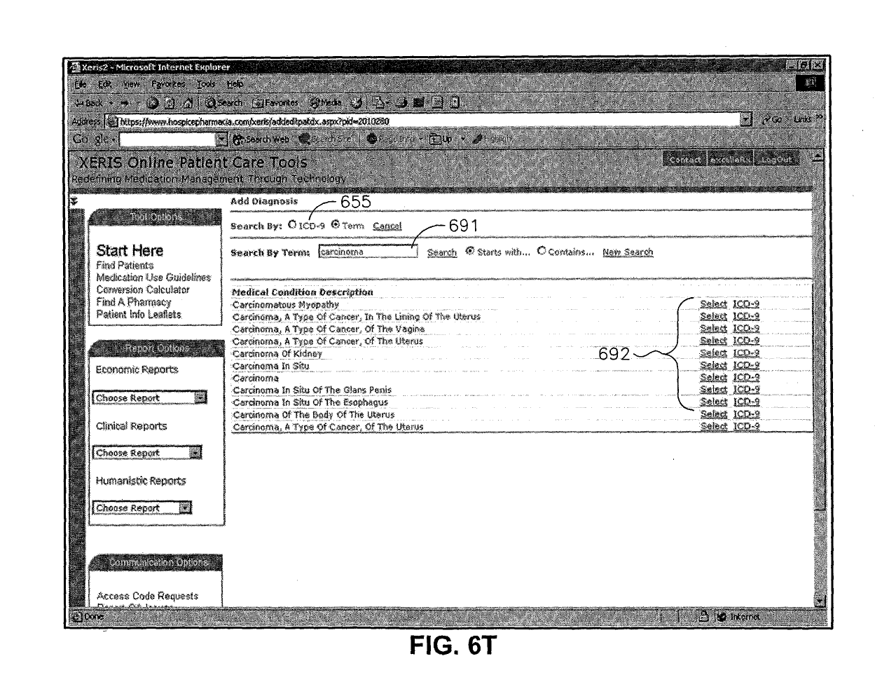

FIGS. 6A-6F show flowcharts illustrating one embodiment of the invention where certain caregivers, such as a hospice, use caregiver interface site 200 to collect and/or exchange patient information using a centralized network such as network system 100. In particular, in accordance with one aspect of the present invention, one embodiment can be used to distinguish between long-term care and homecare patients while entering patient profile online using network system 100.

FIG. 6A is a flowchart illustrating an overview of this process. As shown, a caregiver logs in to network system 100 in step 602. The caregiver may log in using any electronic user interface including personal computer 180 or PDA 170 and gain access network system 100. As noted earlier, caregiver interface site 200 provides a secure and convenient interface between the caregiver and network system 100. Thereafter, a new patient can be added in step 604. In one embodiment, after entering the patient information, such as the last name and/or SSN, it is possible to check whether the patient exists in the system already. This feature ensures avoiding adding redundant profile or having two incomplete patient profiles for the same patient. After entering the basic patient information, a group and/or team of caregivers responsible for particular medical indications and assigned to work with the patient being profiled can be chosen in step 606. Also, a type (i.e., a general diagnosis into which patients are broken down such as heart; lung; cancer aids formulary; and other) is chosen in step 608. In step 610, a patient status as to whether the patient is a long-term care patient is chosen. If in step 612, the patient is determined to be a long-term care patient, the patient profile is directed to long-term-care admission site of the present invention, as shown in step 614. If the patient is not a long-term-care patient, the patient information is entered in step 616. In step 618, specific patient demographic information is entered. Further, the admission date is entered for the patient in step 620. If necessary, any comments can be entered in step 622. Thereafter, in step 624, the patient profile entered is saved. Data representing this patient profile is sent to SQL database, as shown in step 626.

As described further below, the term (i.e., written description associated with a particular ICD-9 code) or ICD-9 information, shown in steps 628 and 640, respectively, is entered to the patient profile. Thereafter, the patient admission confirmation screen is shown in step 650. As noted earlier, the patent profile contains all relevant medical data. As such, any allergy information relating to the patient can be considered in step 652. As described further below, the allergy information is added in step 654. Further, any profiled medication information relating to the patient is considered in step 666. As described further below, the profiled medication information is added in step 668. Also, any diagnosis information relating to the patient can be considered in step 680. As described further below, the diagnosis information is added in step 682.

In FIG. 6B, step 654 of FIG. 6A is illustrated in greater detail. As noted earlier, information contained in a patient profile includes information relating to the patient's allergies. In accordance with one aspect of the present invention, this information is added and/or edited in the patient profile. In one embodiment as illustrated in FIG. 6B, any information relating to patient allergies can be entered manually or automatically using a drop down menu. For instance, in step 656, it is determined whether a patient allergy exists in the drop down menu. If the patient allergy does not exist in the drop down menu, it can be searched several ways. For instance, search criteria can be entered in step 657. Some of the search criteria may be classified by a class, ingredient, and/or product. Thereafter, a list of allergy products is displayed. In step 659, one of the allergy products is chosen from the list.

Upon selecting the allergy, its severity can be chosen in step 660. In step 661, the type of reaction of the allergy can be chosen. In accordance with one aspect of the present invention, in step 662, the patient profile is then updated to reflect the recent addition and/or edit relating to the allergy. Furthermore, data representing this update is sent also to SQL database, as shown in step 663. Thereafter, allergy is added in step 654′.

In FIG. 6C, step 682 of FIG. 6A is illustrated in greater detail. As noted earlier, the patient profile may contain information relating to diagnosis. In step 683, a new diagnosis for the patient is chosen. As shown, in step 684, a search method is decided. The diagnosis search can be by ICD-9 code as shown in step 686, or by term names as shown in step 687. Thereafter, chosen diagnosis is profiled in step 682′, as shown in FIG. 6A.

In FIG. 6D, step 668 of FIG. 6A is illustrated in greater detail. As noted earlier, the present invention allows caregivers to profile all medications that are associated with a patient. The medications can be prescribed or over-the-counter brands. Further, the medications can be current or discontinued. In accordance with one aspect of the present invention, all medications relating to a patient are profiled during the admission process for the patient. In step 670, the medication profile process can begin by selecting the medication. Alternatively or additionally, any new medication is profiled by using a search function in step 671. In one embodiment, the search function is used to look up all medications from a comprehensive list. Note that, as shown in step 672, one feature of the present invention allows medications to be searched by using parts of their names. For instance, a medication can be searched based on the letter(s) it starts with or contains. Thus, all medications that start with the letters “oxyco,” for example, can be searched by entering the letters.

The search function proceeds in step 673, at which point all medications matching the search term will appear. Thereafter, a medication is chosen in step 674. After the medication is chosen, any additional information relating to the medication is added to the patient profile. For instance, in step 675, the method and dosage relating to the medication is added to the patient profile. Also, additional directions for use relating to the medication, such as SIG, are profiled in step 676. If necessary, any additional comments relating to the medication is profiled in step 677. This feature is useful for explaining further information about the medication. For instance, using the feature, any particular reason for selecting the chosen medication is added to the patient profile. In step 678, the patient profile is then updated to reflect the transaction relating to the medication being added to the patient profile. Data representing this update is sent to SQL database, as shown in step 679. Thereafter, in step 668′, the profiled medication is added as shown in FIG. 6A.

In FIG. 6E, step 640 of FIG. 6A is illustrated in greater detail. In accordance with one aspect of the present invention, any diagnosis relating to the patient is added to the profile. As noted in FIG. 6A, during the patient admission process all relevant patient information is added to the patient profile. Along with other features, the profiled diagnosis forms the basis of comprehensive database used in the present invention. Any diagnosis relating to the patient during patient admission process is profiled in step 640.

In one embodiment, as shown in FIG. 6E, any diagnosis (or medical condition as it is used in this application) can be searched by using ICD-9 system by code or terms. In step 641, ICD-9 list of codes is chosen to facilitate the searching process. The search for a diagnosis using ICD-9 code proceeds in step 642, at which point the comprehensive list of ICD-9 codes is displayed. In step 643, the list containing parts of ICD-9 can be viewed. If the list shown does not contain an applicable code for a proper diagnosis, additional codes can be viewed in step 644. When a proper code for the diagnosis is in the list, it is chosen in step 645. The chosen diagnosis (or medical condition) is designated as either primary or secondary in step 646. Any additional comments, if necessary, are entered in step 647. In step 648, the patient profile is then updated to reflect the transaction relating to the diagnosis. Data representing this update is sent to SQL database, as shown in step 649.

In FIG. 6F, step 628 of FIG. 6A is illustrated in greater detail. As noted above, any diagnosis (or medical condition as it is used in this disclosure) is searched using ICD-9 system using the list or terms. In step 630, search by the medical diagnosis term can be chosen. Using the medical diagnosis term search function, in step 631, a diagnosis can be searched by using parts of its name. For instance, a diagnosis can be searched based on the letter(s) it starts with or it contains. In step 632, the search term can be entered, and in step 633, the search can proceed. A proper ICD-9 code or term for the diagnosis can be chosen from the search result in step 634. The chosen diagnosis (or medical condition) can be designated as either primary or secondary in step 635. Any additional comments, if necessary, may be entered in step 636. In step 637, the patient profile is then updated to reflect the transaction relating to the diagnosis. Data representing this update is sent to SQL database, as shown in step 638.

FIGS. 6G-6T depict exemplary web pages of several aspects of the embodiment described above, showing caregiver interface site 200 that can be used by caregivers, such as a hospice, to collect and/or exchange patient information using a centralized network such as network system 100. Furthermore, more specific information, including, among others, medications and complementary therapies relating to specific patients can be profiled upon admission and updated using the features of the embodiment.

Caregivers connect to network system 100 via network 10. Upon connecting, the caregivers will be directed to login screen of caregiver interface site 200. The login screen is same as that shown in FIG. 5G. As noted earlier, using this login screen ensures a secure operating environment to authorized caregivers. As shown in FIG. 6G, a caregiver views a list of current patients associated with the caregiver. Note that any other patients not shown on the list, if any, can be quickly searched by using name box 601, SSN box 603, and/or phone box 605. Also, a new patient can be added by entering appropriate information in boxes 601, 603, 605, and clicking on new patient tab 607.

As shown in FIG. 6H, a subsequent page of caregiver interface site 200 includes group/team drop down menu 609, type drop down menu 611, admission date drop down menu 617, and status drop down menu 613 that are used to add additional pertinent information to the patient profile. Other pertinent information relating to the patient, such as gender, race, birth date, etc., can also be added using appropriate drop down menus in patient information area 615. Any other information that is pertinent can be added in new patient comments box 619. FIG. 6I shows an exemplary web page of caregiver interface site 200 that contains the patient profile of a newly admitted patient. Note that the page shows several hyperlinks including, among others, add new allergies link 621, add new diagnoses link 623, and add new profiled medications link 625. Using the links, the caregiver can add allergies, diagnoses, and profiled medications to the patient profile, and to review profiled medications.

For instance, by clicking on add new allergies link 621, the caregiver will be directed to one embodiment of caregiver interface site 200 containing appropriate area for adding new allergy information to the patient profile. FIGS. 6J and 6K show one embodiment of caregiver interface site 200 that can be used to add allergy information to the patient profile. As shown, the embodiment of caregiver interface site 200 for adding allergies has add new allergy drop down menu 627, add allergy severity drop down menu 629, and add allergy reaction drop down menu 639 that can be used to add, new allergy information. Alternatively, a new allergy can be searched by using a search function. For instance, a term can be typed into allergy search term box 651. Note that allergies can be searched by using terms that relate to particular class, ingredient, and/or product, and/or by severity and reaction, by selecting appropriate box in allergy search criteria area 653, 629, and 639. FIG. 6L shows a list of allergies found from the search using all class, ingredient, and product terms. As shown, the term “oxy” was entered into allergy search term box 651. The embodiment of caregiver interface site 200 of FIG. 6L shows all allergies matching the term organized by class, ingredient, and product, respectively. Note that the names of all class, ingredient, and product in the list are displayed in hyperlinks. Thus, a specific item is selected by clicking on its name directly. A severity of the allergy is added by using allergy severity drop down menu 629.

The caregiver can also add diagnosis to the patient profile. As shown in FIG. 6M, the caregiver can search for a diagnosis by selecting either ICD-9 code list or terms used in ICD-9. This option is selected by checking appropriate boxes in search diagnosis area 655.

Further, as noted, the patient profile also includes information relating to medication. The medication information, in accordance with one aspect of the present invention, is added to the patient's profile. FIG. 6N shows one embodiment of caregiver interface site 200 that can be used to add a medication to a patient's profile. As shown, a medication to be added to a patient's profile can be searched by entering a letter(s) in search medication box 667. The features of the present invention allow medications to be searched by using parts of their names. For instance, as shown in FIG. 6O, a medication can be searched based on the letter(s) it starts with or contains by checking appropriate area in search medication criteria area 669. Thus, all medications that start with the letters “oxyco,” for example, can be searched by entering the letters in search medication box 667. The result of search is displayed in medication search result area 681. Note that the names, in medication search result area 681, are displayed in hyperlinks. Thus, a particular medication can be selected by clicking the name directly. FIG. 6P shows the result of one of the medications from FIG. 6O. As shown, medication dosage and method area 688 contains a list of selected medications, organized by the medication's dosage and method. The medications listed in medication dosage and method area 688 are also displayed in hyperlinks. Thus, a particular medication is selected by clicking the medication desired. FIG. 6Q shows one embodiment of caregiver interface site 200 showing the selected medication from FIG. 6P. As shown, once selected, appropriate directions for taking the medication and any additional comments relating to the medication are added to direction/SIG box 689 and comments box 690.

As noted earlier, any diagnosis relating to the patient can be added to the patient profile. Along with other features, the diagnosis for a patient forms the basis of comprehensive databases used in one aspect of the present invention. FIG. 6R shows one embodiment of caregiver interface site 200 that is used to add any diagnosis. As shown in FIG. 6M, any diagnosis can be searched by using ICD-9 system by code list or terms. This option is selected by checking appropriate boxes in search diagnosis area 655. As shown in FIG. 6R, after checking appropriate box in search diagnosis area 655, a search using ICD-9 code or term can be entered in search diagnosis term box 691. If ICD-9 code search is used, as shown, the result of search is organized by ICD-9 code. Note that each ICD-9 code shown has its description next to it. Further note that all ICD-9 codes shown are displayed in hyperlinks. Thus, a code is selected simply by clicking on the code itself. Alternatively, a code is selected by clicking on the link “select” located in select link area 692. FIG. 6S shows one embodiment of caregiver interface site 200 that further includes disease onset data box 695, primary diagnosis box 693 and comments box 694, all of which are used to add more comprehensive information relating to the selected diagnosis.

Alternatively, a search for diagnosis can be done using ICD-9 terms. Thus, as shown in FIG. 6T, a diagnosis can be searched using terms by checking the appropriate box in search diagnosis area 655 and entering a term in search diagnosis term box 691. The result of the search is shown in FIG. 6T. Each medical condition identified in the search is displayed in hyperlink. Thus, a condition can be selected by clicking on the name itself. Alternatively, the condition can be selected by choosing a link in select link area 692. The select link area 692 includes corresponding ICD-9 link for each condition.

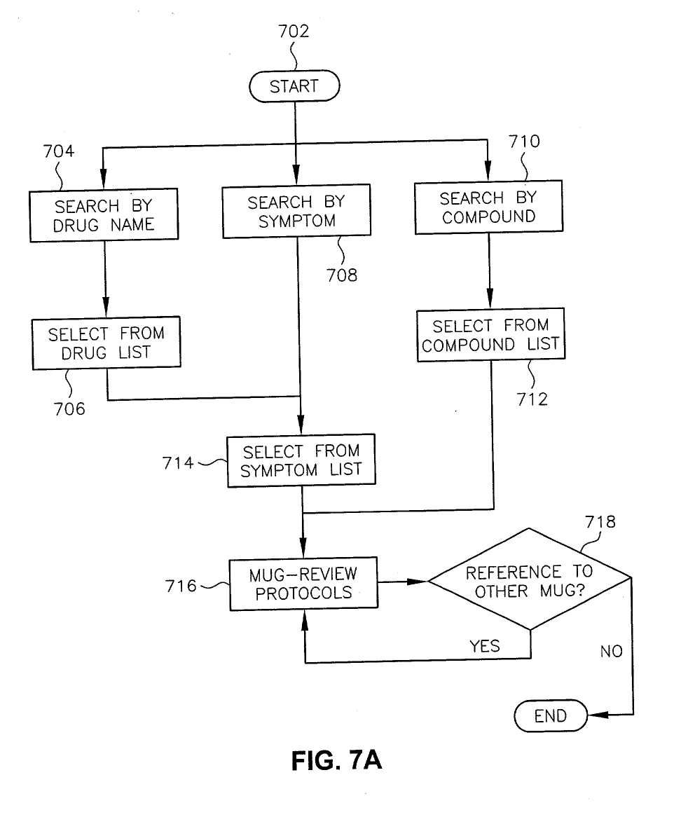

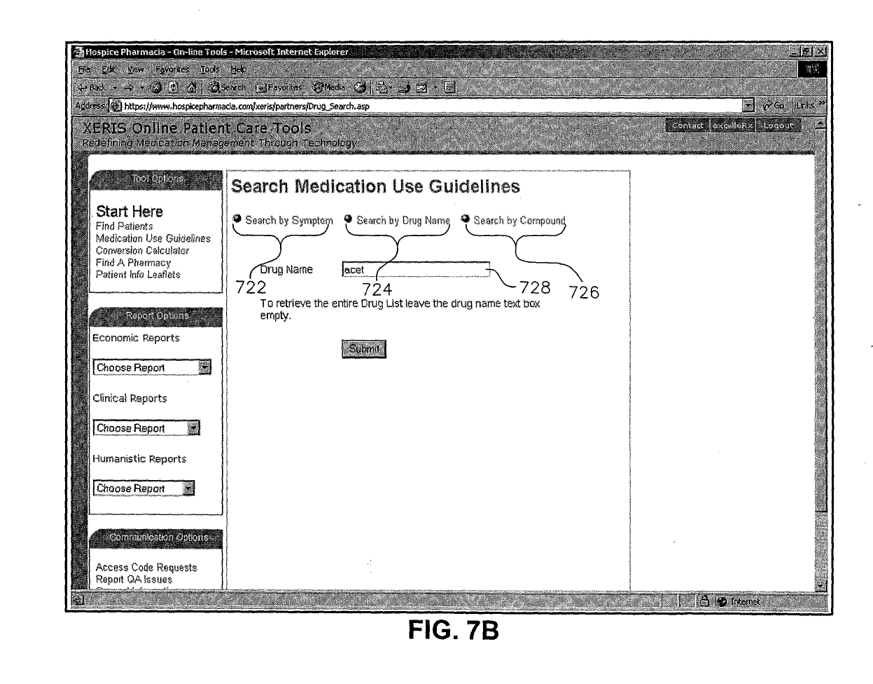

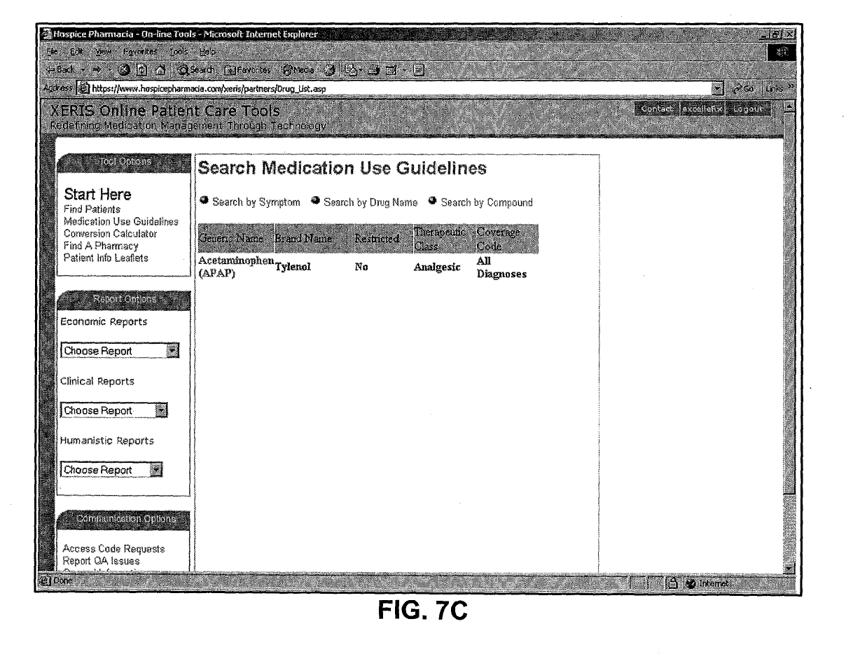

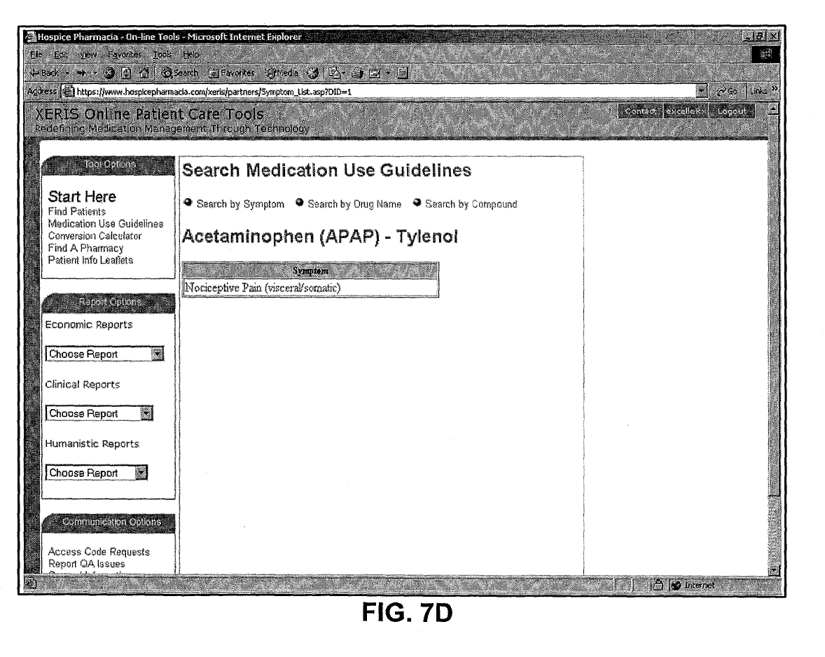

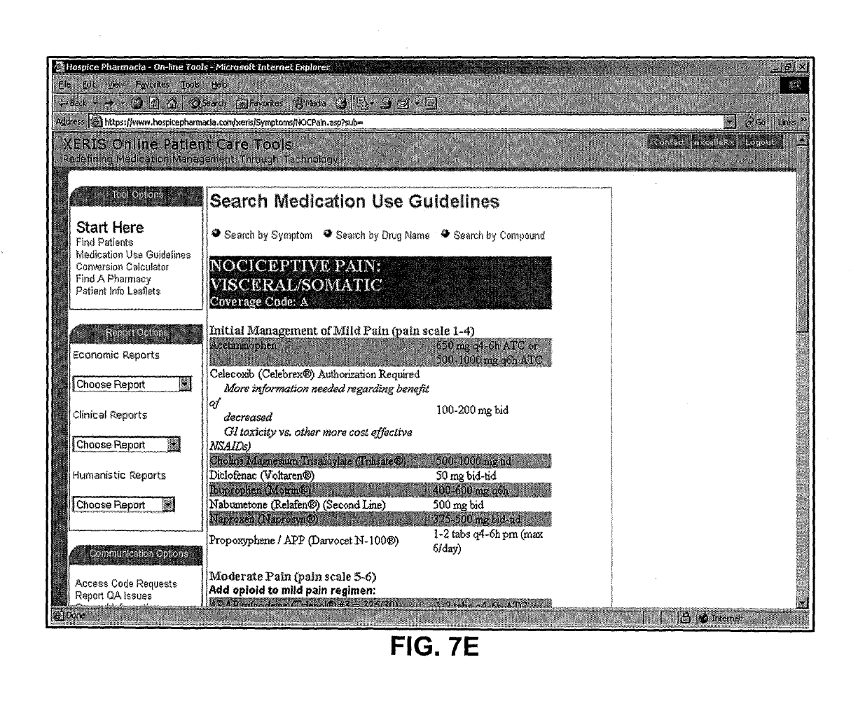

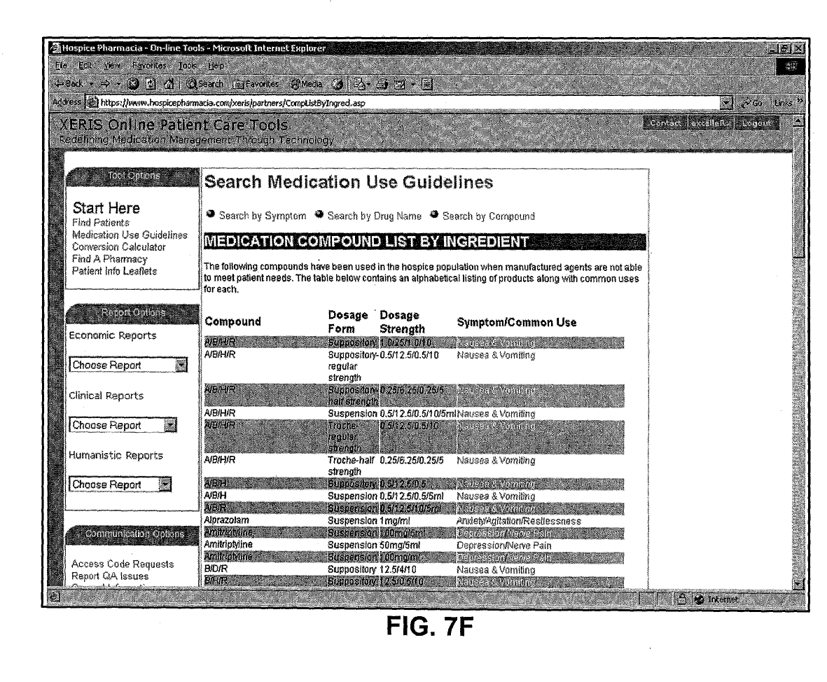

FIG. 7A shows a flowchart illustrating one embodiment of the present invention in which certain caregivers use caregiver interface site 200 to collect and/or exchange patient and/or medication information using a centralized network such as network system 100. In one embodiment, using caregiver interface site 200, caregivers can access databases 120, 145 especially MUGs database, online remotely via network 10. As noted earlier, MUGs database, in accordance with one aspect of the present invention, contains evidenced-based, clinical trials results of selected medications. In other words, MUGs database contains evidence-based, peer-reviewed, step-care protocols relating to all relevant aspects of the selected medications. The relevant aspects include, among other things, the efficacy, safety including any side effects, long term effect, cost information, etc.

As shown in FIG. 7A, MUGs database can be searched several ways. In one embodiment, MUGs database logically follows a relational database structure, and as such, the database can be searched using look up fields search terms and display matching step-care protocols. In step 704, MUGS database can be searched by drug name. Using this feature, matching treatment protocols of the drug can be searched. Thus, if the term “acet” is entered, all drugs that include that term including, for instance, the drug Acetaminophen appear in a list. In step 706, any one of the drugs from the list can be selected. Thereafter, in step 714, the selected drug can be further defined by matching with specific symptoms. This step will ensure that a specific protocol will be matched with the selected drug. Alternatively, in step 708, MUGs database can be searched by symptom. Furthermore, in step 710, MUGs database can be searched by compound. This feature is especially useful to those interested in finding out data relating to a specific compound. For instance, using this feature all medications containing “Amitriptyline” can be searched. From the list of medications containing Amitriptyline, one particular medication can be chosen in step 712.

It is apparent from the foregoing description that a treatment protocol can be searched using any information relating to the protocol. Once the protocol is selected, it can be viewed in step 716. In step 718, the selected treatment protocol is evaluated further to determine whether the protocol references other relevant protocols. If it is determined in step 716 that there are other relevant protocols referenced in the selected protocol, those protocols can also be viewed.

FIGS. 7B-7F depict exemplary web pages of the embodiment described above, showing caregiver interface site 200 that can be used by caregivers to collect and/or exchange patient and/or medication information using a centralized network such as network system 100. As noted, MUGs database can be searched by symptom, drug name, or compound. FIG. 7B, in accordance with one aspect of the present invention, shows search by symptom link 722, search by drug name link 724, and search by compound link 726. A caregiver can search MUGs database by using any one of these links 722, 724, 726. After selecting the desired link, a term can be entered in search box 728. As shown, the term “acet” is entered in search box 728.

FIG. 7C shows the result of search using drug name that starts with the term “acet.” As shown, a drug name Acetaminophen is retrieved. Note that names of drug are shown in hyper links. Thus, as shown, the name Acetaminophen is also shown in hyperlinks, which can be linked to another page containing comprehensive information related to Acetaminophen. By clicking the Acetaminophen name itself, the caregiver can view more information pertaining to that medication. Here, as shown, only one drug “Acetaminophen” matched the search criteria. More information relating to this drug, such as common symptoms treated by the drug, can be viewed by simply clicking on the name. FIG. 7D shows the list of common symptoms treated by Acetaminophen including Nociceptive Pain. Alternatively, as noted, MUGs database can be searched by using compound link 726. FIG. 7F shows the list of all medication compounds searched by using compound link 726. As shown, the list contains an alphabetical listing of products along with dosage forms, dosage strengths, and symptoms/common uses for each. If one of the compounds is chosen, symptoms treated by medications using that compound are displayed, as shown in FIG. 7D. By clicking on the name of symptom shown in FIG. 7D, a caregiver can access treatment protocols relating to that symptom. FIG. 7E shows an exemplary web page containing a protocol relating to Acetaminophen. As shown, the protocol includes comprehensive information relating to Acetaminophen, including, links that can be “drilled down” further to see in-depth detail relating to Acetaminophen such as other possible medications, relative cost comparison information for the other medications, and dosages.

In accordance with another aspect of the present invention, the drug dispensing process can be facilitated using remotely located Access to Medication (ATM) machines 190. Currently, there are many pharmacies that stay open for business 24 hours a day, 7 days a week. However, for many patients who require prescription medications, the extended hours of these pharmacies do not necessarily help as the pharmacies are prohibited from dispensing medications without the requisite prescriptions. Obviously, there is a need to facilitate drug dispensing process 24 hours a day, 7 days per week, for those patients who require prescription medications.

ATM machines 190 serve this purpose by dispensing prescriptions and/or medications 24 hours a day, 7 days a week. ATM machines 190 include a medication dispensing portion that can be used by an authorized caregiver to dispense a medication to an authorized party remotely. The authorized caregiver may be, in accordance with the present invention, one who has the authority to prescribe and/or dispense medications. In one embodiment, ATM machines 190 are operatively coupled to network system 100 using network 10. ATM machines 190 are located through out diverse geographic areas to serve medication needs of many patients. For instance, the machine 190 may be located in an inpatient facility, hospice, pharmacy, or hospital. ATM machine 190 may even be located in non-healthcare facilities such as a super market, shopping mall, street corner, train station, or even in patient's own home. Using ATM machines 190, patients can have access to requisite prescriptions for medications at any time and any place, so long as federal, state, and local regulations and laws permit dispensing medications from such locations.

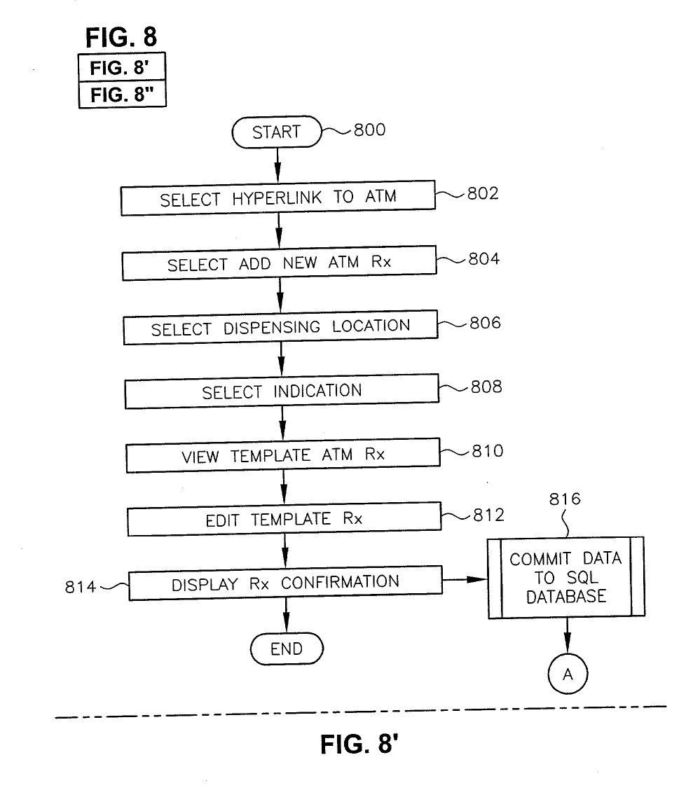

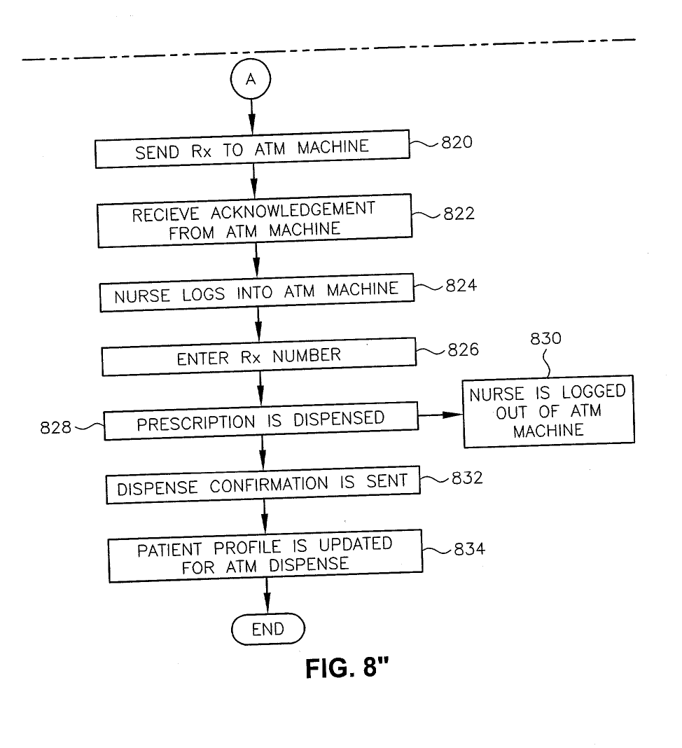

FIG. 8 shows a flowchart illustrating one embodiment of ATM machines 190 that can be used to facilitate drug dispensing process. In step 800 a caregiver, such as a physician, can log on to network system 100 via network 10. Using caregiver interface site 200, the caregiver can select a hyperlink that takes the caregiver to ATM machines options in step 802. There, the caregiver may add new ATM prescription for a patient in step 804. The caregiver may also select a particular location for dispensing the prescription and/or a medication in step 806. An indication (or medical indication) for treatment can be selected in step 808. In step 810, a template for the ATM prescription form is viewed. Also, the template can be written out and/or edited in step 812. In step 814, the prescription information is displayed for confirmation. Data representing this transaction can be sent also to SQL database, as shown in step 816.

In step 820, the prescription is sent to the selected ATM machine 190. As noted, the ATM machine 190 was previously selected by the caregiver in step 806. After the prescription is transmitted to the ATM machine 190, the machine 190 sends back an acknowledgment notice to the caregiver in step 822. In step 824, a nurse logs into the ATM machine 190, for example, using an access code and password. In step 826, the prescription is selected. The prescription form is dispensed in step 828. As noted, the prescription form can be printed using any printing device installed in the ATM machine 190 or any other printing devices coupled to ATM 190 such as printer 185. After the prescription is printed and dispensed, a dispense confirmation is sent to the caregiver and to main system 110 over network 10. Using the dispense confirmation, the patient profile is updated in step 834. The nurse then logs out of the ATM machine 190 in step 830.

In accordance with one aspect of the present invention, patients can check their own patient profiles using network system 100. Using personal computer 180 or PDA 170, the patients can log in to network system 100 via network 10. Thereafter, using caregiver interface site 200, patients can access their own profiles. In particular, the patients can obtain in-depth information relating to specific medications or other medication related materials. Note that other types of caregiver such as physicians can use this feature to review their patients' profiles.

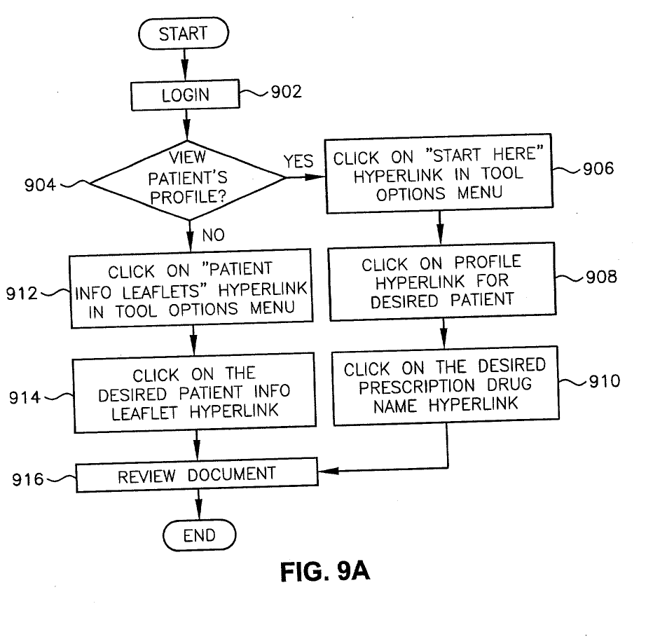

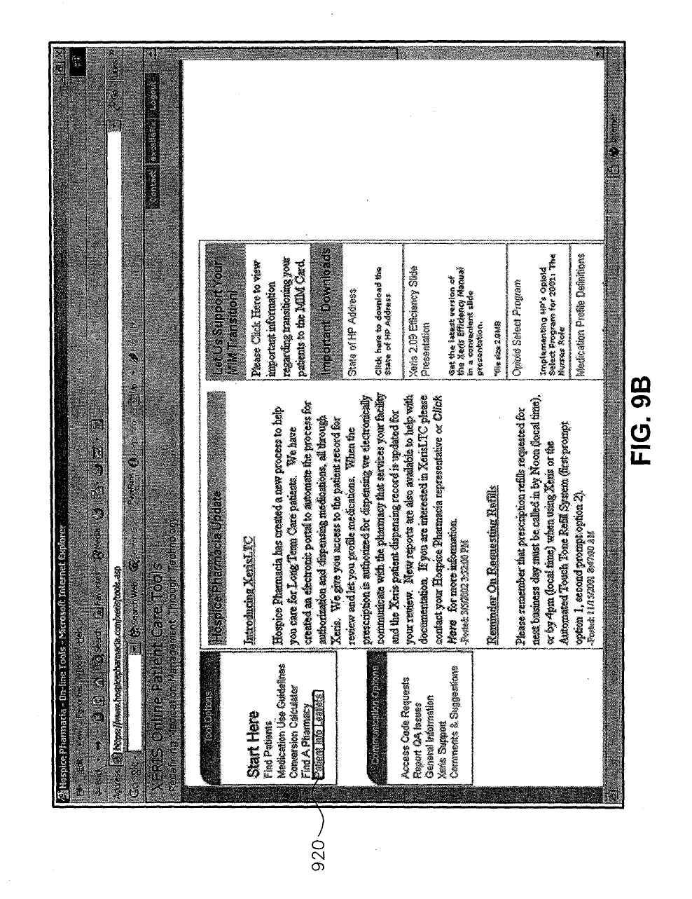

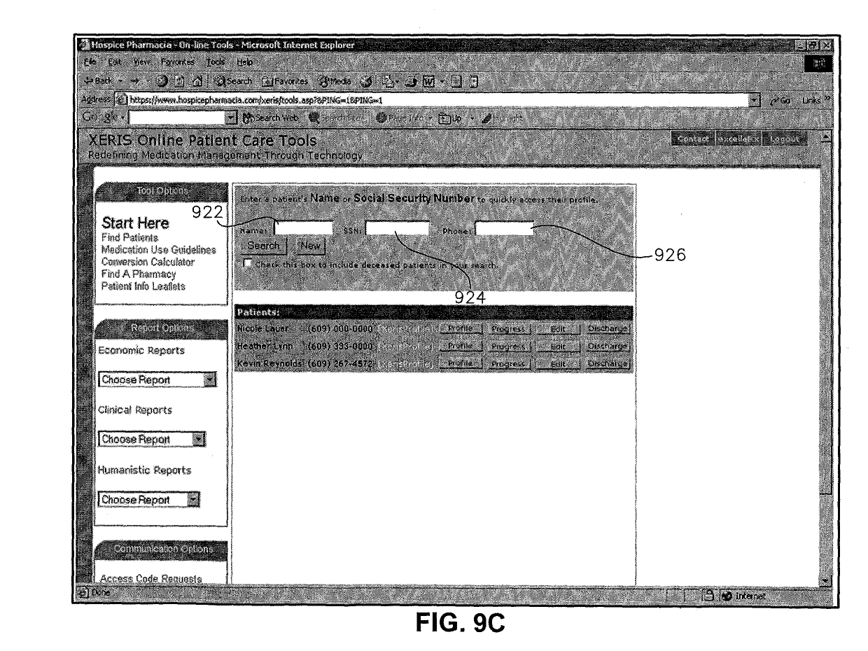

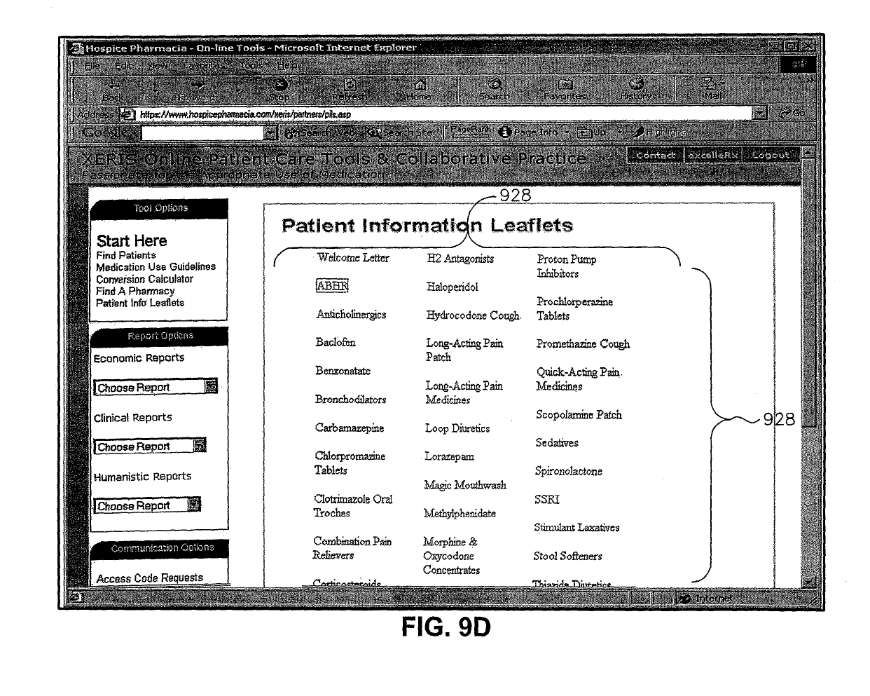

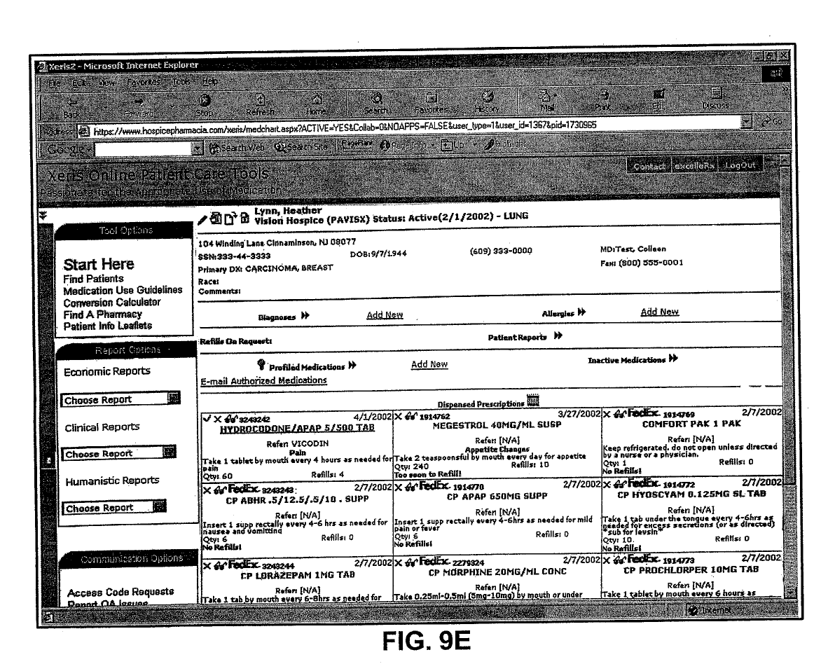

FIG. 9A shows a flowchart illustrating the above process where patients can log in to network system 100 and check their own medical profile or pertinent medical information. In step 902, the patient caregiver logs in to network system 100. Using caregiver interface site 200, the caregiver can choose to view its own patient profile in step 904. If viewing patient profile is desired, the caregiver can proceed by selecting appropriate option in step 906. For a physician caregiver (or any other non-patient caregiver), it is possible to view profiles of all patients associated with the caregiver in step 908. Thus, if desired, the physician caregiver may select a patient profile, and thereafter select any medication profile associated with the patient profile in step 910.

Alternatively or additionally, patients (or other types of caregivers) may view in-depth information relating to specific medications or other related materials in step 912. After choosing this option to view in-depth information, a list containing the topics of all relevant materials is shown. In step 914, the patient can choose any one of the topics. The chosen topic is then displayed in step 916 at which point the caregiver may review the document.

FIGS. 9B-9E depict exemplary web pages of the embodiment described above, showing caregiver interface site 200 that can be used by patients to obtain relevant medication information and their own medical history profiles. More specifically, the exemplary web pages show that patients can access network system 100 and review in-depth information relating to specific medications. Patients can also access their specific medication history including information relating to their current medication status.

As shown in FIG. 9B, after logging onto network system 100, a patient or caregiver obtains desired information by clicking on Patient Info Leaflets 920. As shown, many pertinent highlights relating to the patient and/or medication are displayed on caregiver interface site 200, which can be reviewed by the patient. As noted earlier, a caregiver can view, using this feature, a patient profile of any one of the patients associated with the caregiver. FIG. 9C shows a list of patients that are associated with the caregiver. As shown, the caregiver may search any particular patient by using name box 922, SSN box 924, and/or phone box 926.

FIG. 9D shows one embodiment of caregiver interface site 200 displaying patient information leaflets. As shown, in patient information leaflets topic area 928, names of all pertinent medical indications (or diagnoses) or medication are listed in alphabetical order. The names of each medical indication (or diagnoses) and medications are displayed in hyperlinks using HTML (or XML), and as such, the patient can access more information that may be placed in the same page or other entirely different pages by simply clicking on appropriate hyperlinks. FIG. 9E shows a patient profile showing the status of currently dispensed prescriptions for a patient. Thus, using this feature, the patient can, among other things, track its current medication information.

In accordance with the present invention, one embodiment of caregiver interface site 200 is used by certain caregivers such as a hospice to obtain more control over the pharmacotherapy care for its patients. Using network system 100, which acts as centralized electronic portal, for example, the hospice can process and request authorization and dispense medications remotely. For instance, using network system 100, a hospice can authorize medications covered under the hospice benefit service (i.e., those medications covered under the hospice's prescription and medication plan for which the hospice typically pays the medication provider on a per diem basis per patient), thereby eliminating the need for staff to review the long-term care bills for non-authorized charges. The hospice caretaker, using network system 100, can access main system 110 and receive medication management and consultation relating to its patients.

The hospice can access patient records for review, and can also profile (i.e., recording the detailed description of what a patient is taking) medications as needed. When a prescription is authorized for dispensing, a centralized contact center equipped with main system 110 electronically communicates with a pharmacy that services the hospice, and furthermore, the patient dispensing record is automatically updated so it can be reviewed by the hospice caretaker and the pharmacist. All participating hospices then can use these features from their remote sites using remote system 140, personal computer 180, or PDA 170.

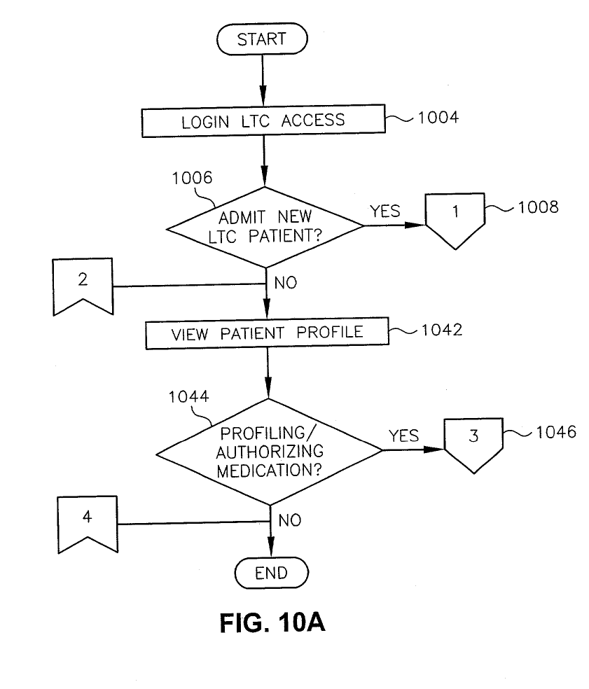

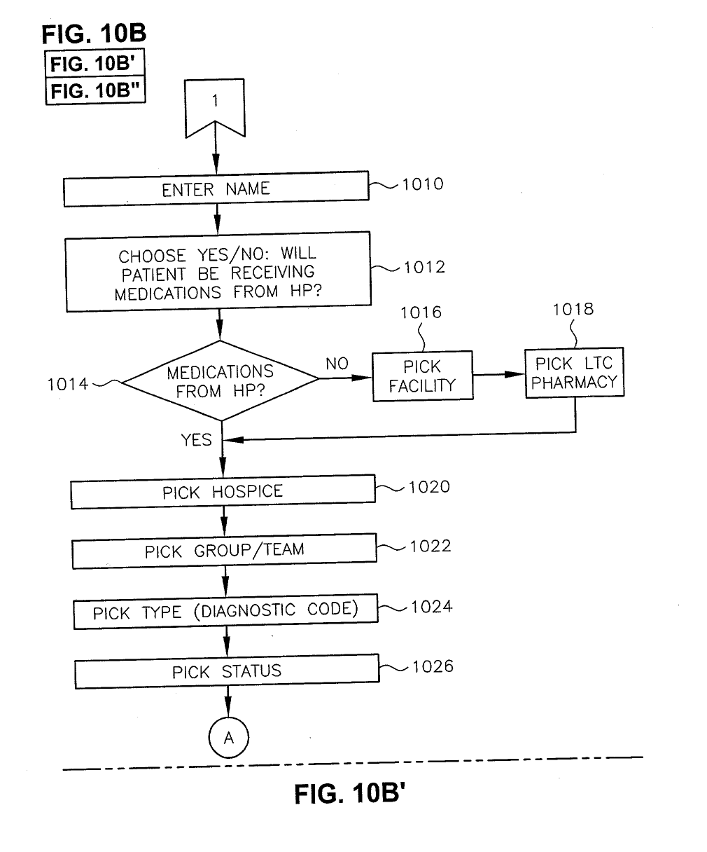

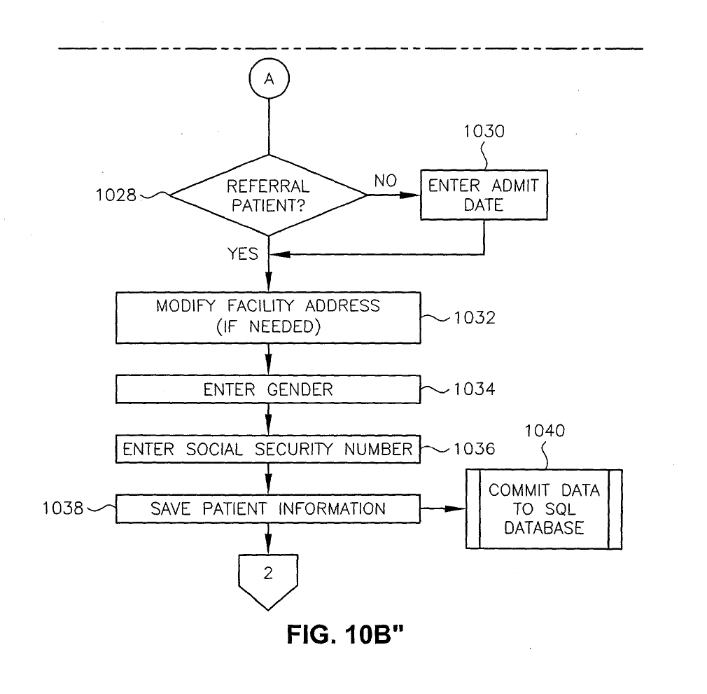

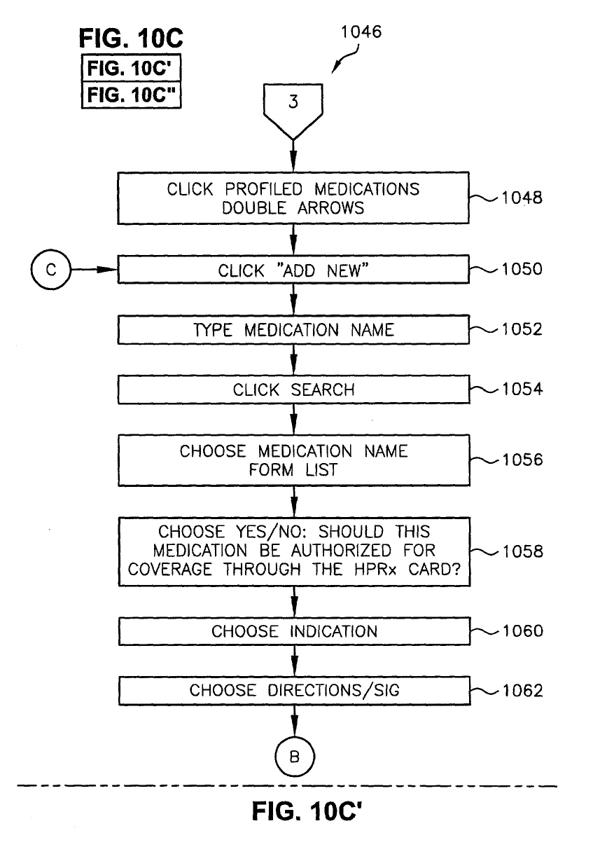

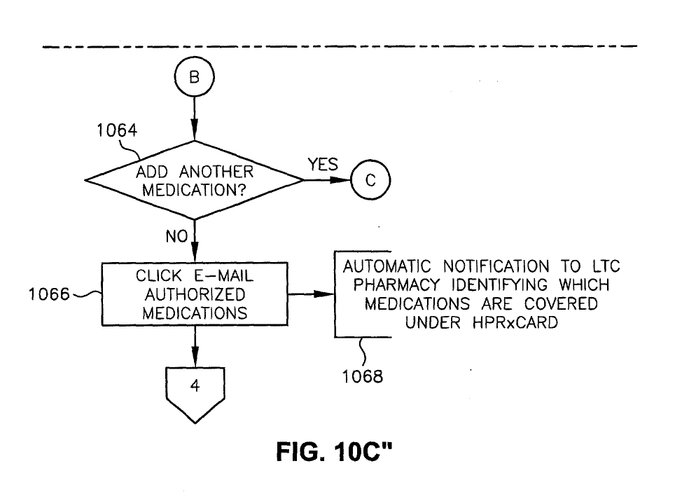

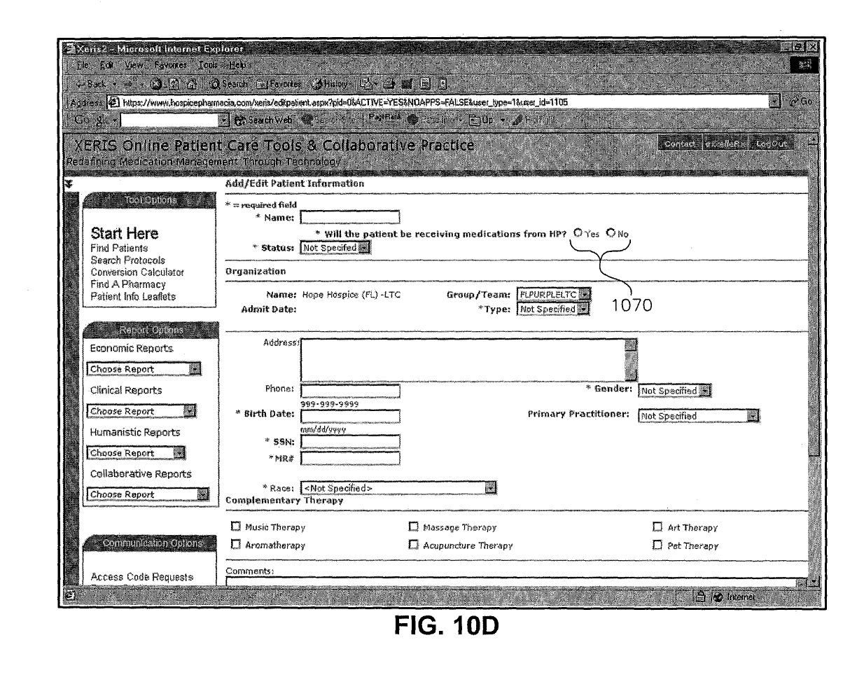

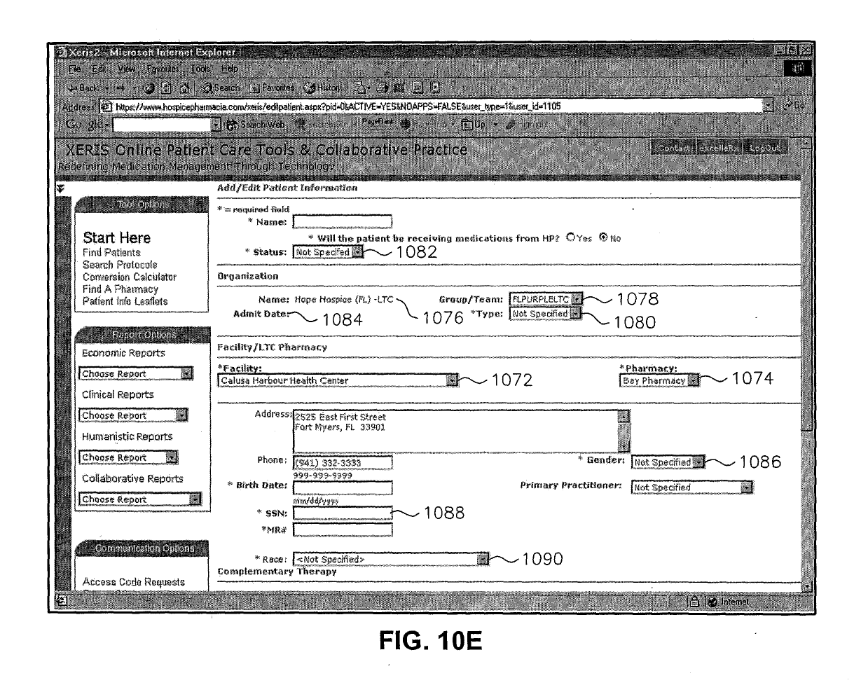

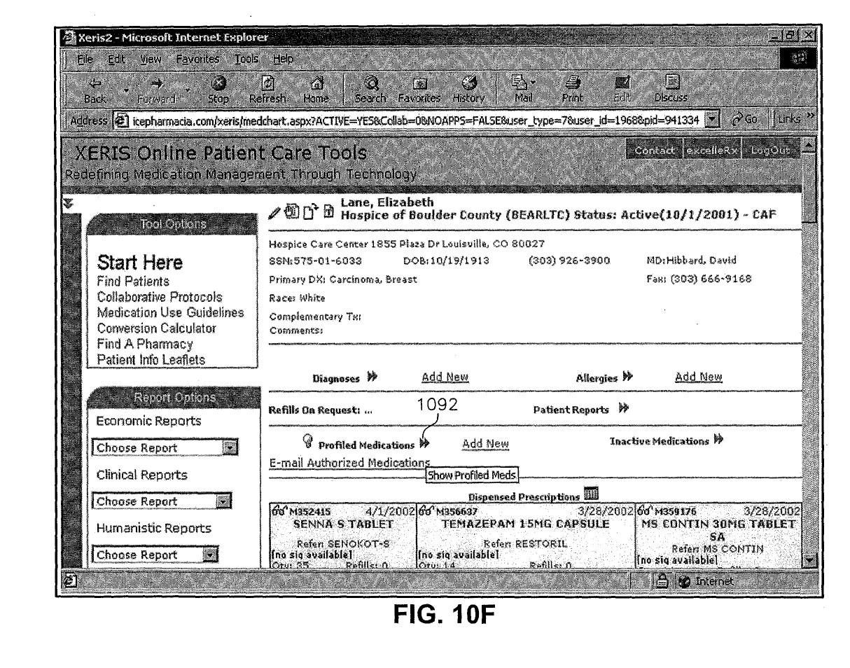

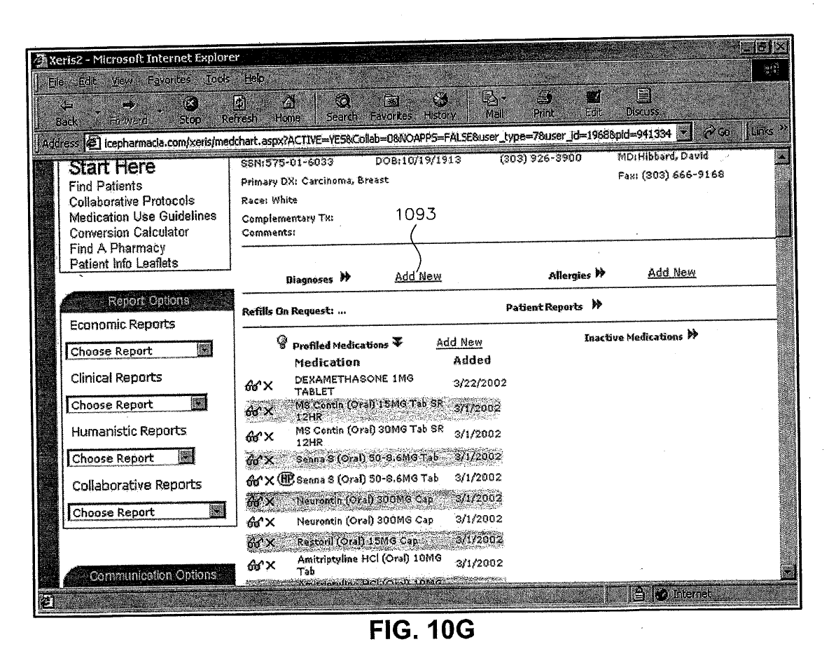

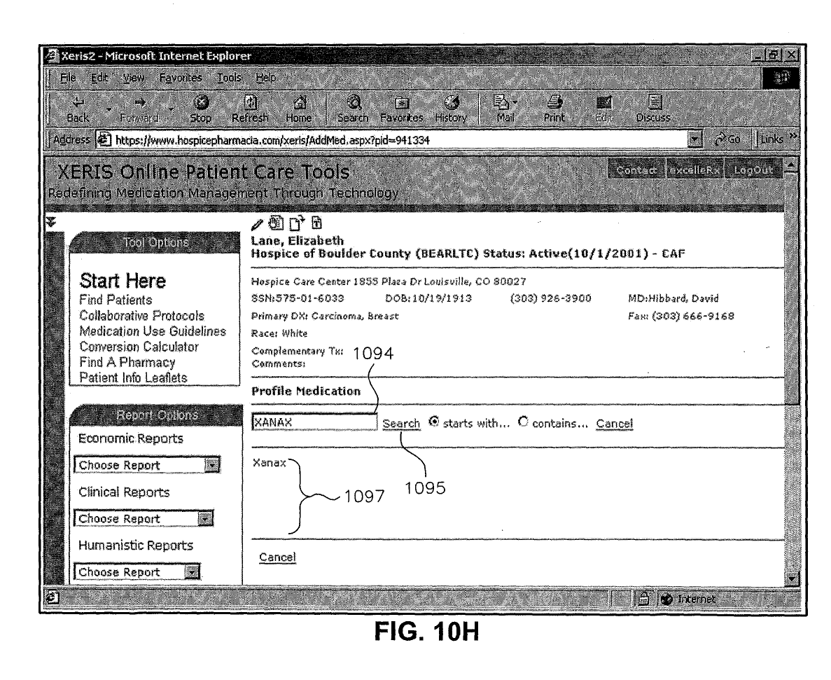

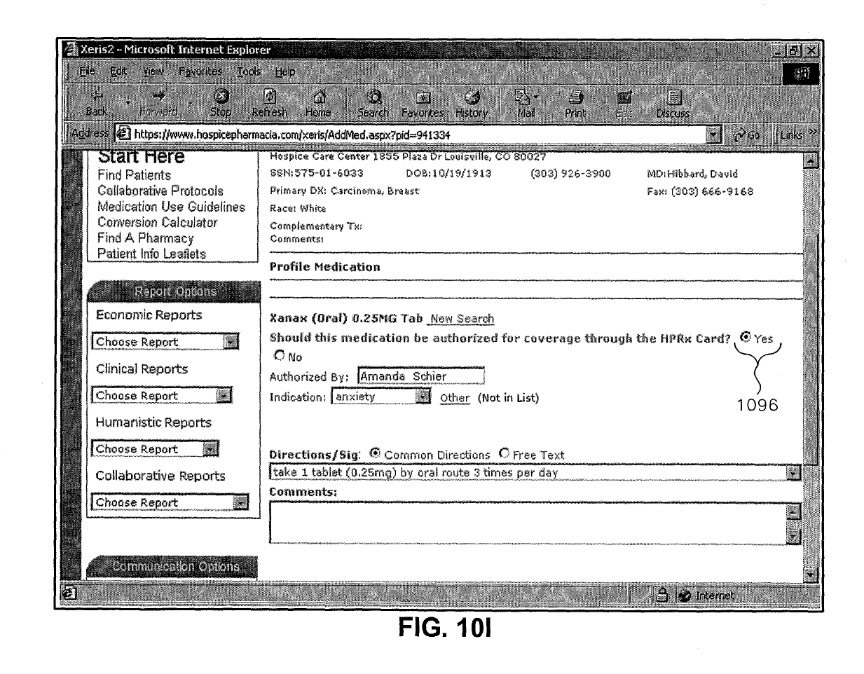

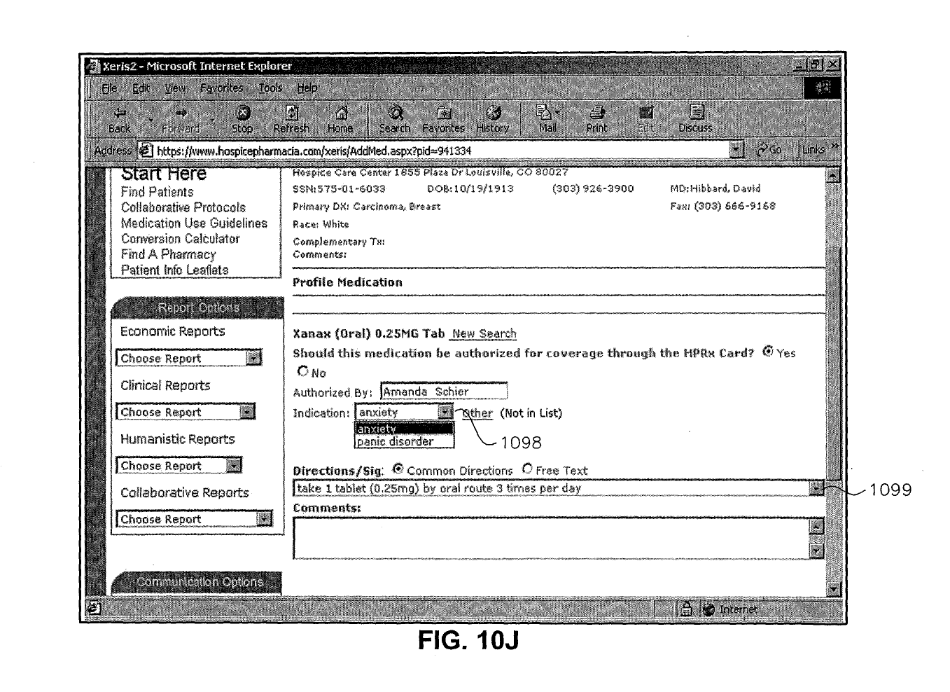

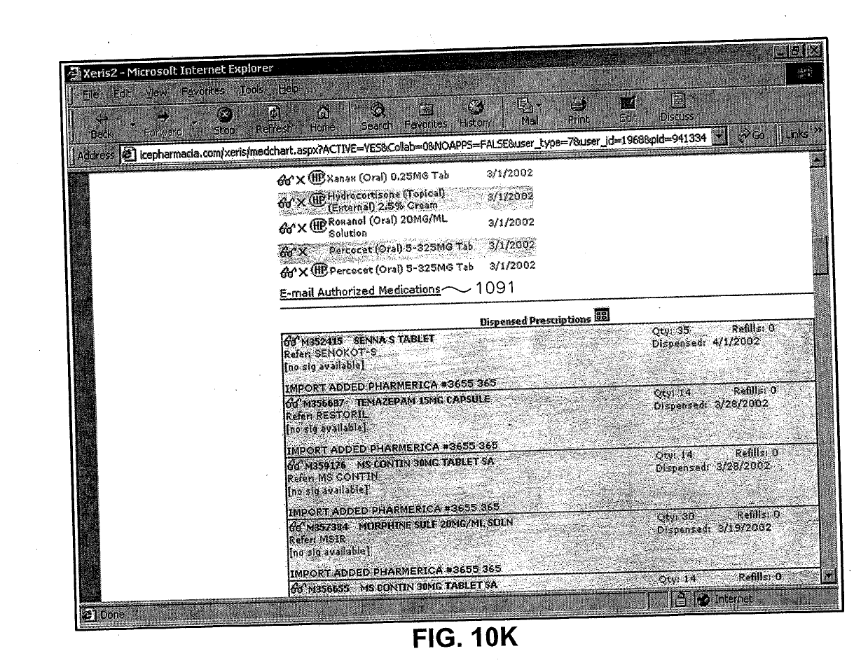

This process of managing pharmacotherapy control online is illustrated in FIGS. 10A-10C. As shown in FIG. 10A, a hospice logs in to network system 100 in step 1004. In step 1006, the hospice decides whether to admit a new long-term-care patient. As described further below and shown in step 1008, a long-term-care patient, if admitted to the hospice, is profiled (i.e., recording all relevant patient data pertaining to patient profile). In step 1042, the patient profile can be viewed. In step 1044, it is determined whether any medication associated with the patient needs to be added to the patient's profile. If necessary, as described further below and shown in step 1046, any medication can be added to the patient profile.

In FIG. 10B, step 1008 of FIG. 10A is illustrated in greater detail. A caregiver can use this process to record and create a profile for its long-term-care patient. As shown, in step 1010, the patient's name is entered. The caregiver can choose whether the patient will be receiving medications from a hospice in step 1012. If it is decided that the patient will not receive medications from a hospice, in step 1014, a facility such as a health center or pharmacy is chosen for the patient in step 1016. Additionally, a long-term-care pharmacy can be chosen for the patient in step 1018. Whether the patient receives medications from a hospice or otherwise, a hospice is chosen for the patient in step 1020. Note that the hospice chosen in step 1020 need not supply the medications to the patient. A pharmacy chosen in step 1018 can supply the medications for the patient instead. If desired, the chosen hospice of step 1020 can supply the medications for the patient.

After a hospice is chosen, a group and/or team (i.e., those persons in a care facility responsible for the care of a particular group of patients) is entered in step 1022. Also, a type (i.e., a general diagnosis into which patients are broken down such as heart; lung; cancer aids formulary; and other) can be entered in step 1024. A status (such as, active or to be active, also referred to as a “referral” status) can be chosen in step 1026. In step 1028, it is determined whether the patient is a referral patient. For a referral patient, the admission date can be entered in step 1030. Thereafter, the facility address can be modified, if necessary in step 1032. In step 1034, the gender of the patient is entered. Likewise, the SSN for the patient is entered in step 1036. In step 1038 the patient information is saved. Data representing this transaction is then sent to SQL database, as shown in step 1040.

In FIG. 10C, step 1046 of FIG. 10A is illustrated in greater detail. As noted earlier, any medication can be added to a patient profile for a patient. In step 1048, the caregiver may view all profiled medications of the patient. If desired, a new medication can be profiled in step 1050. The name of a new medication is entered in step 1052. In accordance with one aspect of the present invention, a new medication can also be searched in step 1054. Using databases 120, 145, a new medication can be searched using its generic name, trade name, and the like, for example. In step 1056, from the list of medications shown (i.e., the result from search), one medication can be chosen. In one embodiment, the caregiver can select, as shown in step 1058, an option indicating that the medication be authorized for coverage under a prescription card provided by the caregiver (or other authorized caregiver). An indication is chosen in step 1060. An indication represents, as noted earlier, a diagnosis or medical condition. Thus, the indication represents the diagnosis for which the medication is targeted. Thereafter, a direction (i.e., SIG) for use of the medication is chosen in step 1062.

Another medication can be added in step 1064. If more medication will be added, the caregiver can repeat the process of adding additional medical indications to the patient profile, starting from step 1050. In step 1066, a message such as an e-mail message indicating authorized medications is sent to staff and to a dispensing pharmacy. In accordance with one aspect of the present invention, an automatic notification, identifying which medications are covered under the prescription card provided by the caregiver (or other authorized caregiver), is sent to a long-term-care pharmacy in step 1068.

FIGS. 10D-10K depict exemplary web pages of the embodiment described in FIG. 10A above, showing one embodiment of caregiver interface site 200 that can be used by a caregiver, such as a hospice, to obtain more control over the pharmacotherapy care for its patients. FIG. 10D shows the embodiment of caregiver interface site 200 that is used to collect information relating to a patient. Note that caregiver interface site 200 shown has Hospice Pharmacia (HP) medication status area 1070 that can be used to check whether the patient will be receiving medications from Hospice Pharmacia or another such a prescription and medication provider. As shown in FIG. 10E, the caregiver may select a particular facility where patient is located for the patient using facility drop down menu 1072. Also, the caregiver may select a particular long-term-care pharmacy for the patient using pharmacy drop down menu 1074. The caregiver may also pick a hospice for the patient using hospice box area 1076. As shown, the embodiment of caregiver interface site 200 further includes group/team drop down menu 1078, type drop down menu 1080, status drop down menu 1082, admit date drop down menu 1084, gender drop down menu 1086, SSN drop down menu 1088, and race drop down menu 1090, all of which form the basis of comprehensive patient profile, in accordance with one aspect of the present invention.

As noted, the patient profile includes a comprehensive list relating to associated medication profiles. FIG. 10F shows one embodiment of caregiver interface site 200 that can be used to add and/or edit patient medication profiles. Profiled Medications Link 1092 is a link that takes a caregiver to view all profiled medications. From caregiver interface site 200 shown in FIG. 10G, the caregiver can add new medications by clicking add new link 1093 and record the date each medication was added. Using the embodiment of caregiver interface site 200 shown in FIG. 10H, the caregiver may search medications to be profiled. The caregiver can search for a medication by entering the name of the medication in medication name box 1094, followed by clicking medication search link 1095. The result of search showing all medications containing the search term is listed in medication list area 1097. Note that all names of medication displayed in medication list area 1097 appear in hypertext links, which can be further drilled down, as explained above. Thus, the caregiver can select a particular medication by clicking on the name of the medication.

Shown in FIG. 10I, the caregiver can control whether to have the medication authorized for coverage through a prescription card (e.g., HPRx Card) issued by the caregiver. This process can be done by checking a box in prescription card authorization area 1096. As shown in FIG. 10J, the caregiver can choose appropriate indications that are treated by the selected medication. This can be done by selecting appropriate indications from indication drop down menu 1098. Also, appropriate directions/SIG for the medication can be selected by using direction/SIG drop down menu 1099. As shown in FIG. 10K, a list of authorized medications can be e-mailed to long-term-care pharmacy identifying which medications are covered under the prescription card. This can be done by clicking on e-mail authorized medications link 1091.

In accordance with the present invention, one embodiment is provided where prescribing physicians can use caregiver interface site 200 to refer patients over network system 100. A caregiver, who may also be the treating physician, can view all relevant and specific records of a patient. Using the information, the caregiver can make intelligent, informed referrals of the patient to other facilities.

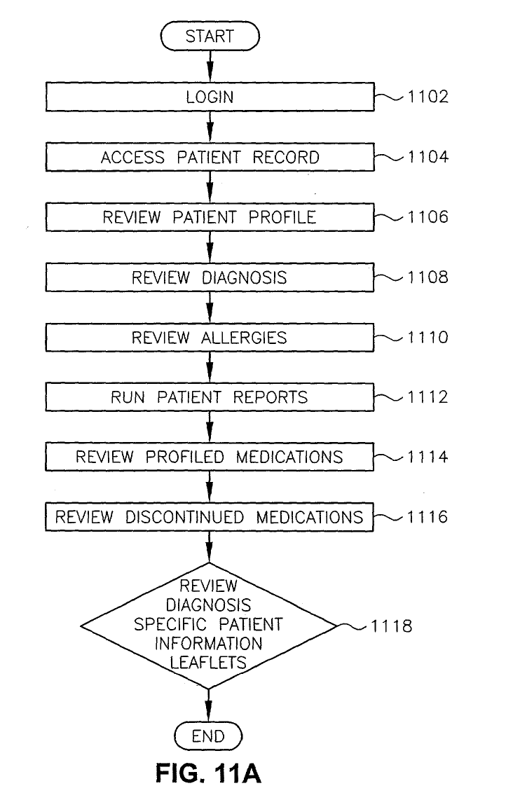

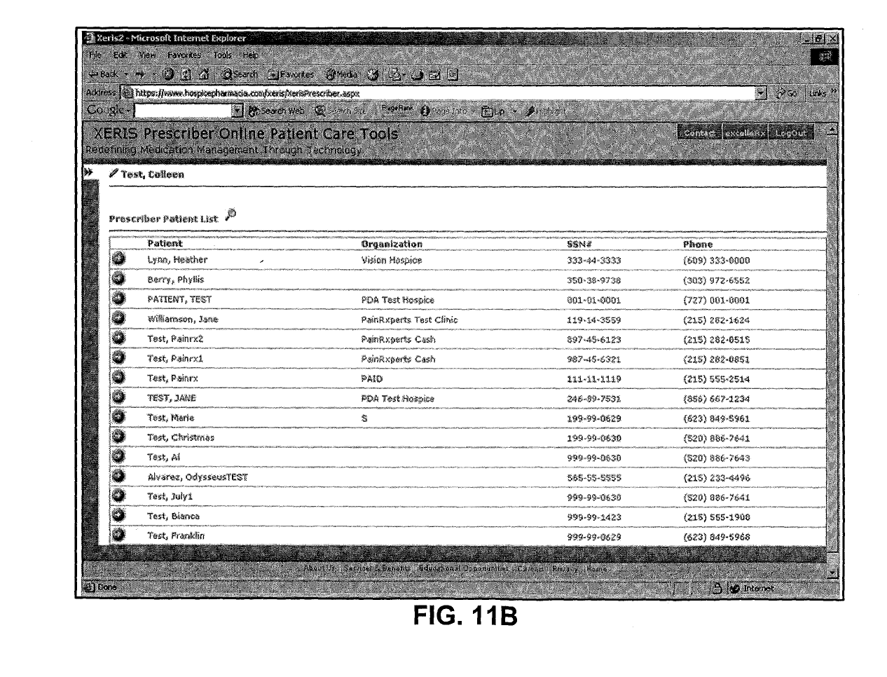

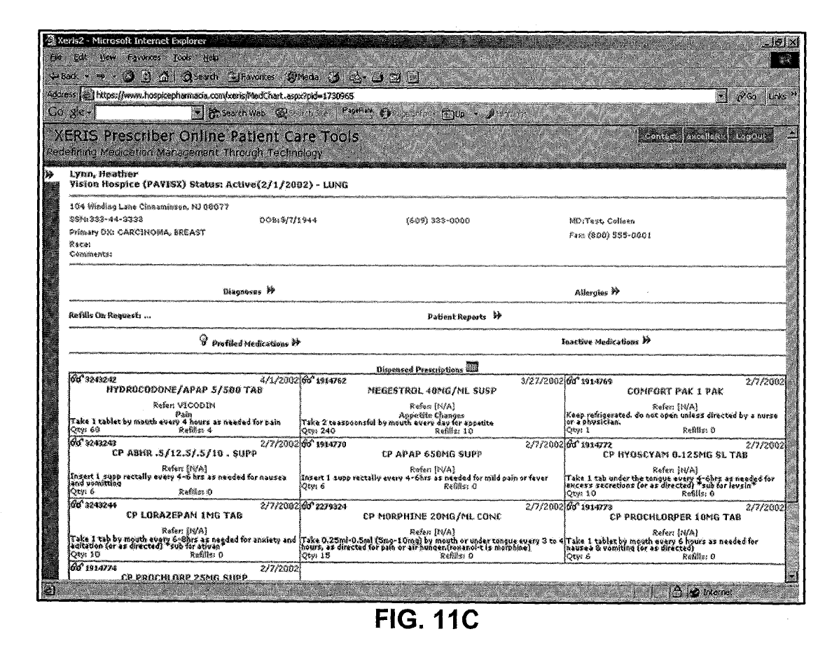

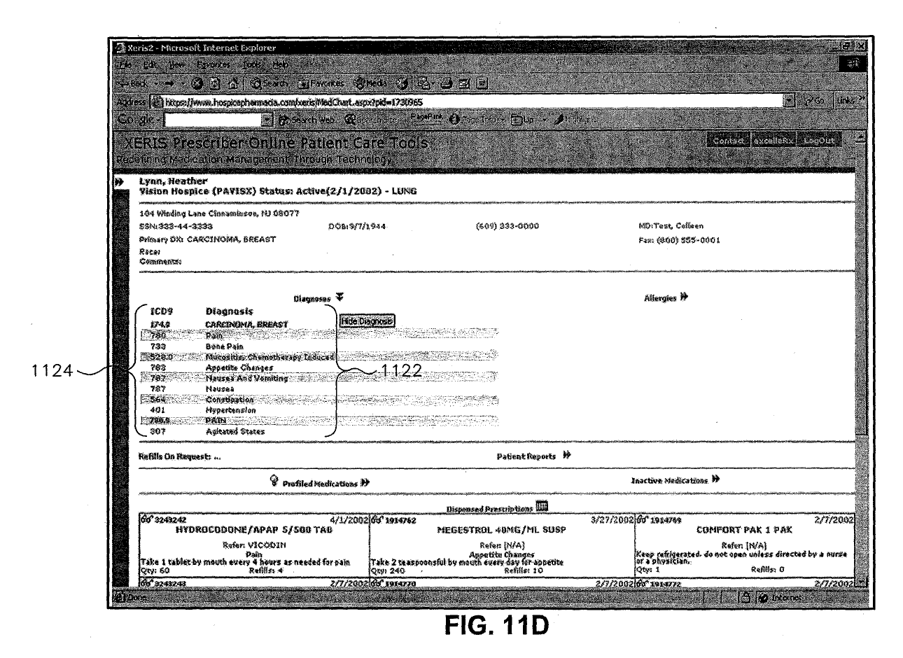

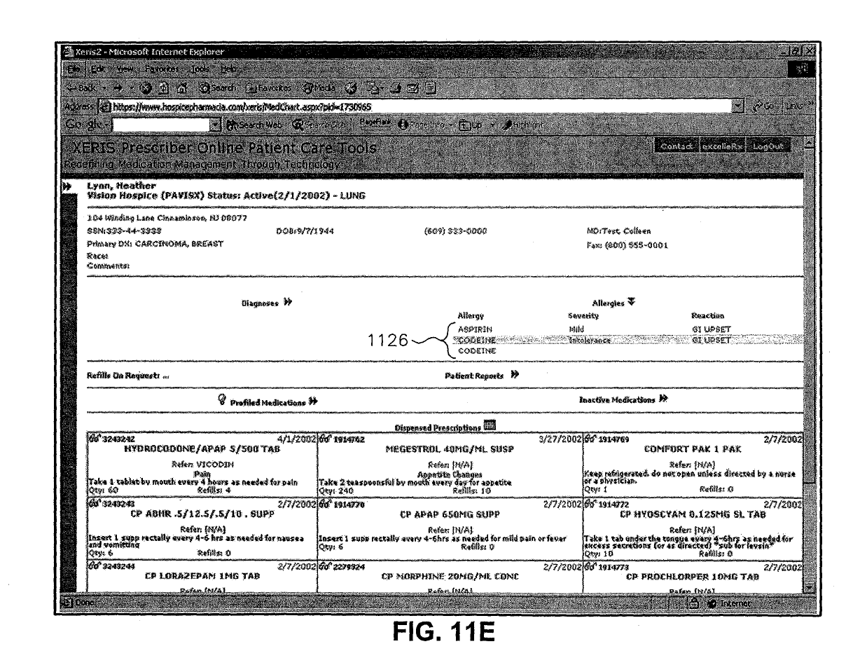

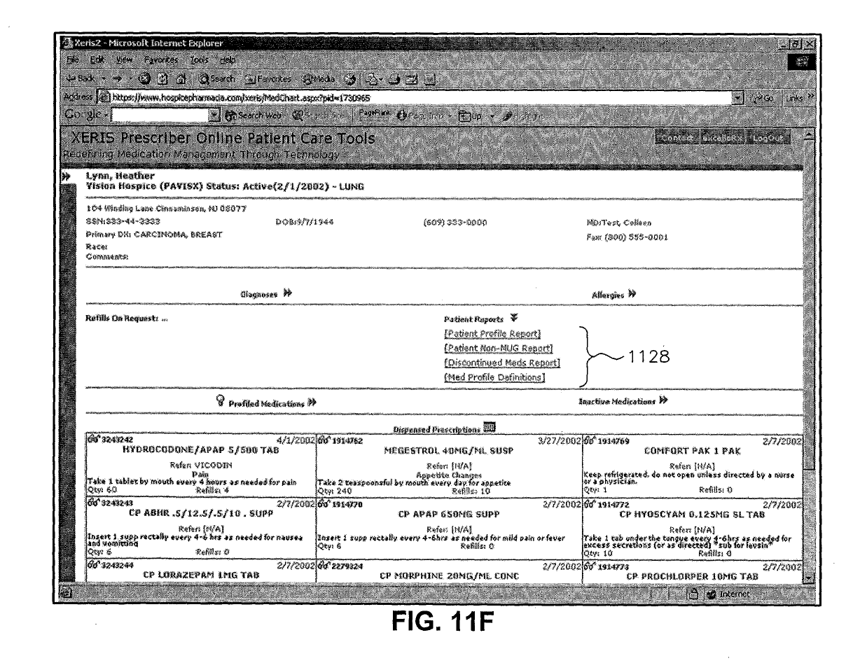

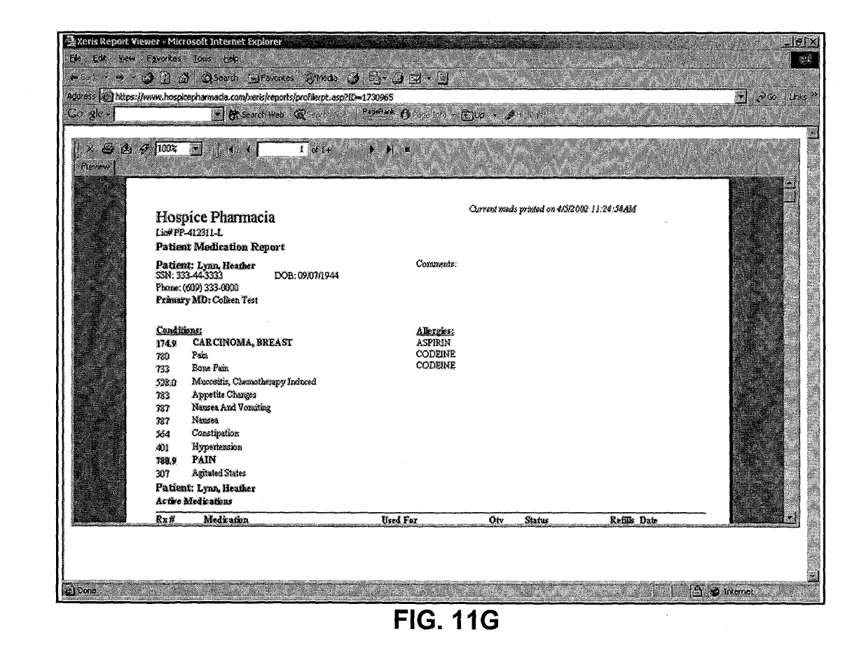

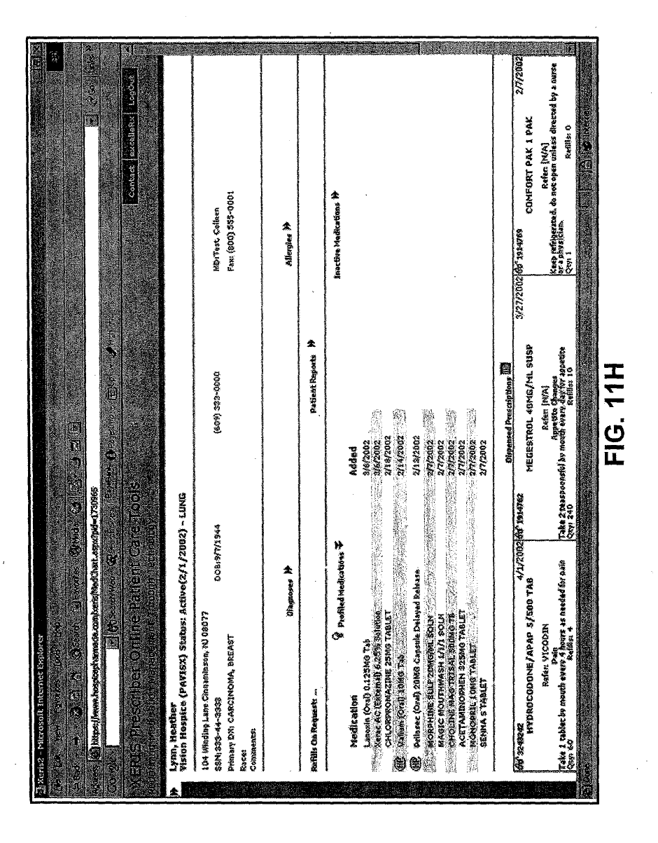

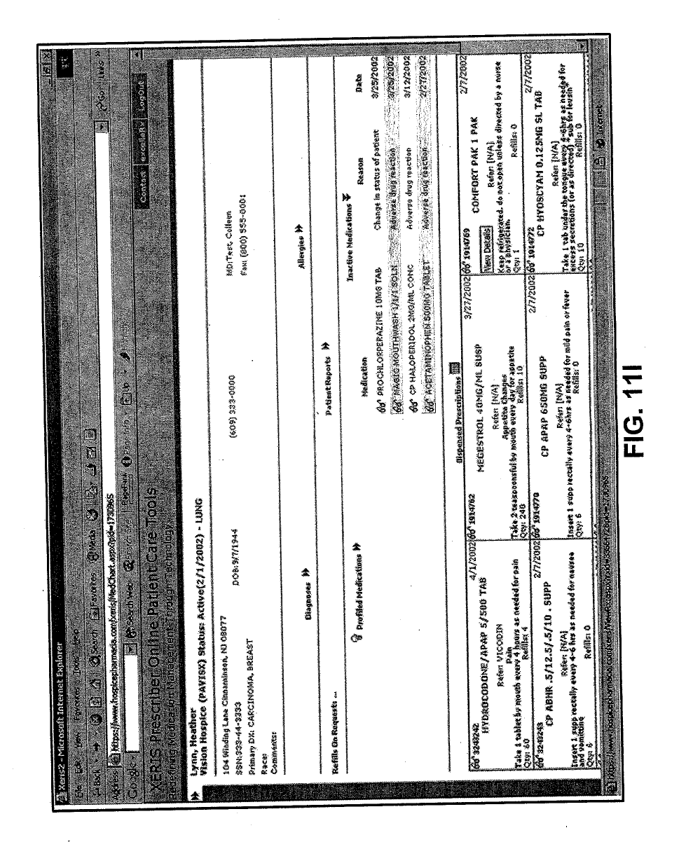

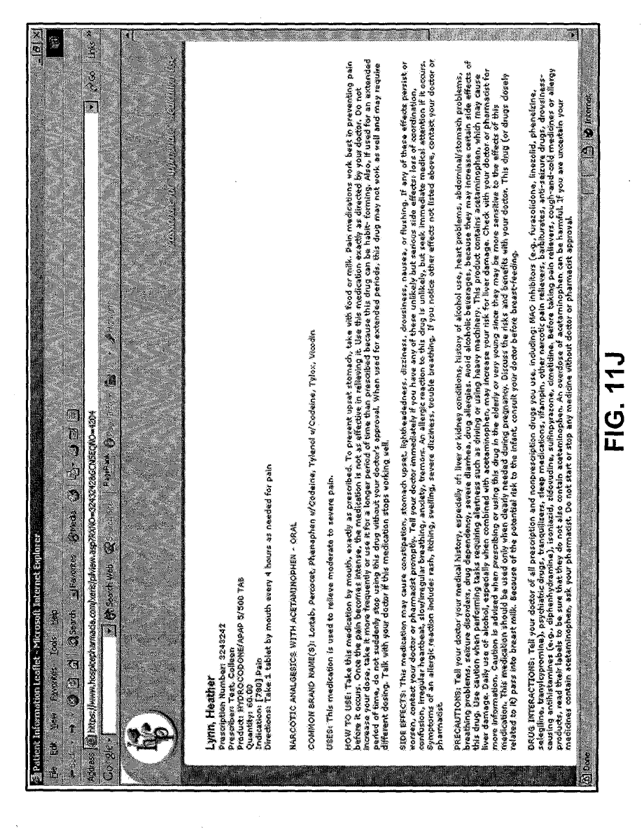

FIG. 11A shows a flowchart illustrating one embodiment of the process as described above. In step 1102, the caregiver logs in to network system 100. The caregiver will then be directed to a caregiver interface site 200. In step 1104, the caregiver may access records of all patients associated with the caregiver. In step 1106, the caregiver may review the patient profile of a selected patient. If desired, the caregiver may view diagnoses of the selected patient in step 1108. Also, if desired, all information relating to the patient's allergies can be viewed in step 1110. In step 1112, a patient report can be generated. The patient report contains selected information from the patient profile as directed by the user of the present invention. Some of the information may include, for instance, allergies, medical conditions, medications, economic data, etc. All profiled medications associated with the selected patient can be reviewed in step 1114. In step 1116, all discontinued medications associated with the selected patient can be reviewed. In step 1118, any diagnosis information such as, specific patient information leaflets, directed specifically at the patient can be reviewed. The information may include, for instance, relevant medication information associated with the patient.

FIGS. 11B-11J depict exemplary web pages of the embodiment described above, showing caregiver interface site 200 that can be used by caregivers to refer patients over network system 100. FIG. 11B shows a list of patients that are associated with the caregiver from step 1104. As noted earlier, the caregiver can review the patient profile for a selected patient. FIG. 11C shows the patient profile of the selected patient, which was chosen in step 1106. If desired, the caregiver can review the diagnosis associated with the selected patient. This is shown in FIG. 11D. As shown, each diagnosis listed in diagnosis list 1122 corresponds to an ICD-9 code listed in ICD-9 list 1124. The caregiver can also view the list of allergies of the patient, shown in allergy list 1126 in FIG. 11E. As noted, the caregiver can run any one of the patient reports shown in patient report list 1128 of FIG. 11F. For instance, a patient medication report that the caregiver ran is shown in FIG. 11G. Also, as noted, the caregiver can review the patient's medication profile. An exemplary web page showing a medication profile of the selected patient is shown in FIG. 11H. Further, the caregiver can also review a list of discontinued or inactive medications for the patient. This is shown in FIG. 11I. Any specific report or information relating to the selected patient, the treatment of the patient, or a medication, if available, can also be reviewed by the caregiver. In accordance with one aspect of the present invention, a report may also relate to several topics including usage of medications, shipping information, prescription use pattern (or the patient), MUGs prescriptions, non-MUGs prescription, controlled substance prescriptions, non-controlled substance prescriptions, etc. A sample report containing specific information relating to use of narcotic analgesics with Acetaminophen is shown in FIG. 11J.

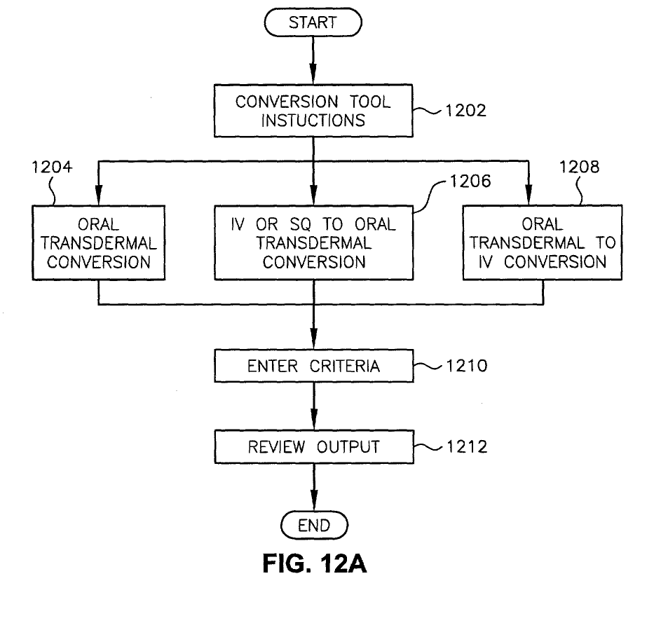

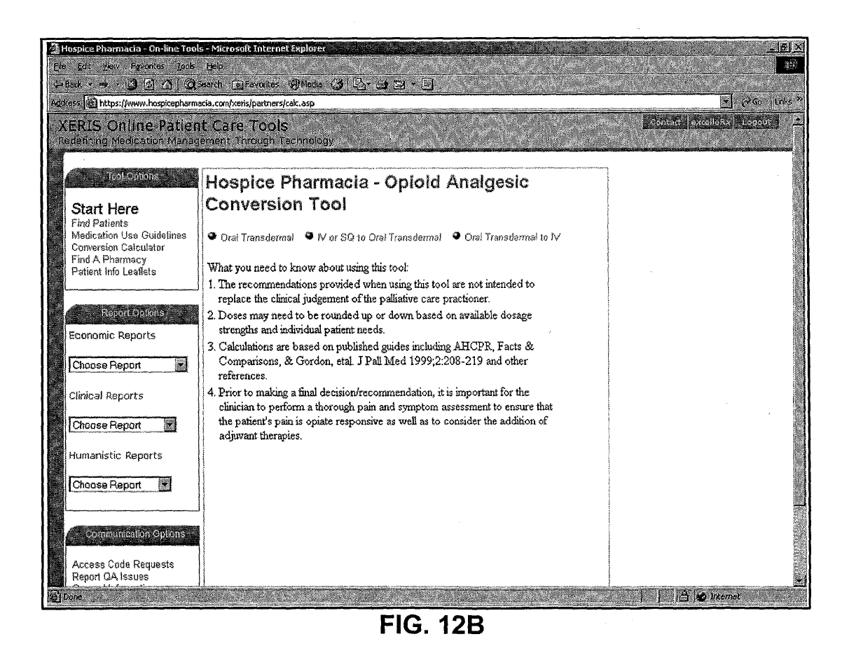

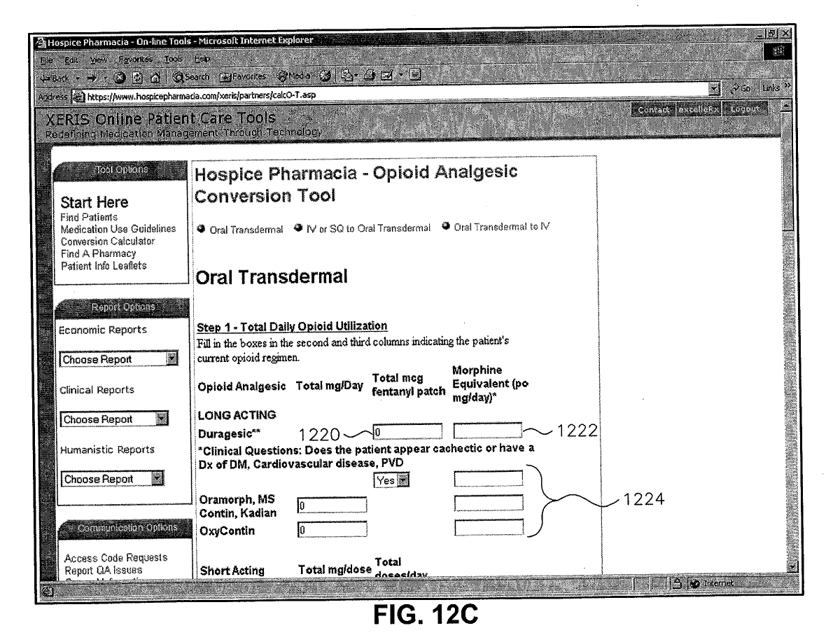

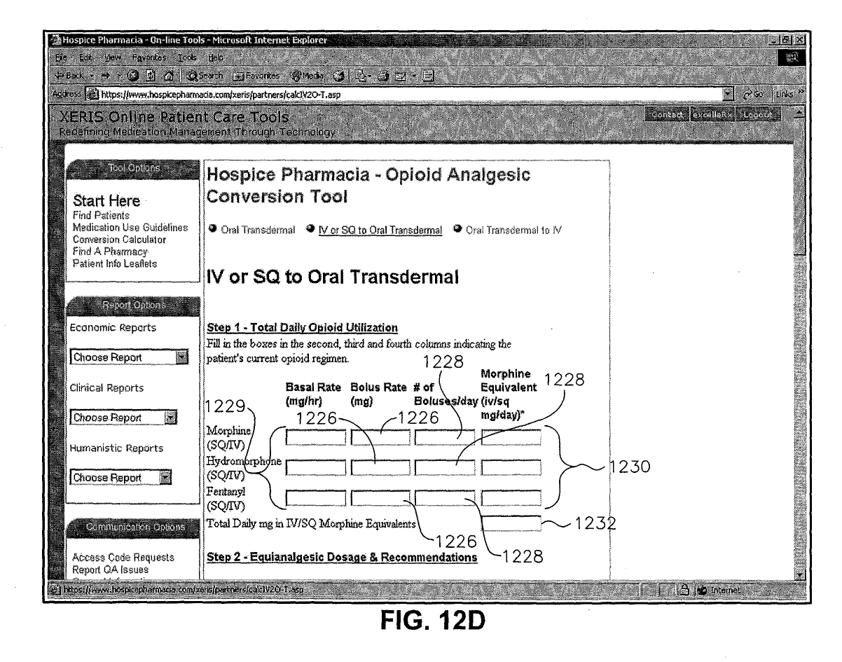

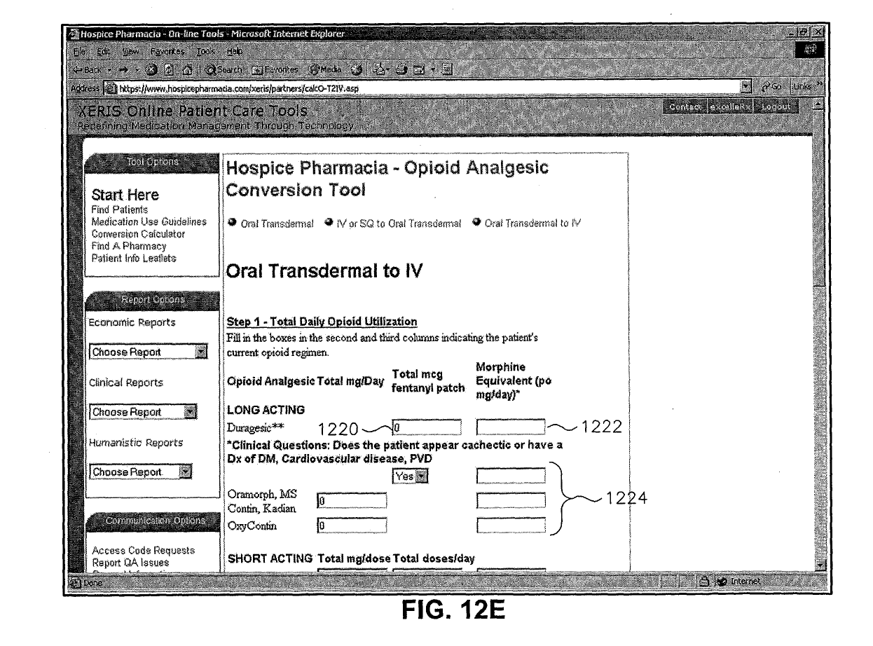

In accordance with another aspect of the present invention, caregivers can use caregiver interface site 200 to identify all levels of certain medications or classes of medications a patient has been taking. In one embodiment, the level of a particular medication or class of medications is determined by converting a medication dosage from one unit (or type) to another unit (or type) or the level of one medication to an equivalent level of another medication. FIG. 12A shows a flowchart of one embodiment illustrating the process of conversion. In step 1202, caregivers can view, shown in one embodiment of caregiver interface site 200, how to use the conversion tool provided in the embodiment. Several units of measurement can be converted using this feature. For instance, in step 1204, oral dosage can be converted to a transdermal dosage and vice versa. Also, in step 1206, intravenous (IV) or subcutaneous (SQ) dosage can be converted to oral or transdermal dosage. Alternatively or additionally, in step 1208, oral or transdermal dosage can be converted to IV dosage. Note that criteria for conversion, such as a conversion factor, can be entered in step 1210. In step 1212, the output of the conversion is reviewed. Using this tool, a user, for example, can measure the total opioids a patient is taking or if taking another pain medicine, an equivalent level of opioids represented by the level of the other medication the patient is taking.

FIGS. 12B-12D depict exemplary web pages of one aspect of the embodiment described above, showing caregiver interface site 200 that can be used by caregivers to convert quickly a medication dosage from one unit (or type) to another unit (or type). FIG. 12B shows one embodiment of caregiver interface site 200 containing conversion tool instructions. FIGS. 12C and 12E show another embodiment of caregiver interface site 200 that includes, among other things, Oral Transdermal conversion tool, an IV or SQ to Oral Transdermal conversion tool, and an Oral Transdermal to IV conversion tool. As shown, there are total mcg box 1220 and equivalency box 1222. The caregiver can fill in total mcg box 1220 and equivalency box 1222 information relating to the patient's current opioid regimen. Once the boxes 1220, 1222 are filled, the converted results are shown in conversion box result area 1224.

Alternatively, FIG. 12D is showing yet another embodiment of caregiver interface site 200 that contains IV or SQ to Oral Transdermal tool. As shown, caregiver interface site 200 of one aspect of the present embodiment includes basal rate box 1229, bolus rate box 1226, number of boluses/day box 1228, and equivalency box 1230. The caregiver can fill in these boxes 1229, 1226, 1228, 1230, which reflect the patient's current opioid regiment, and get the converted result in conversion result box 1232.

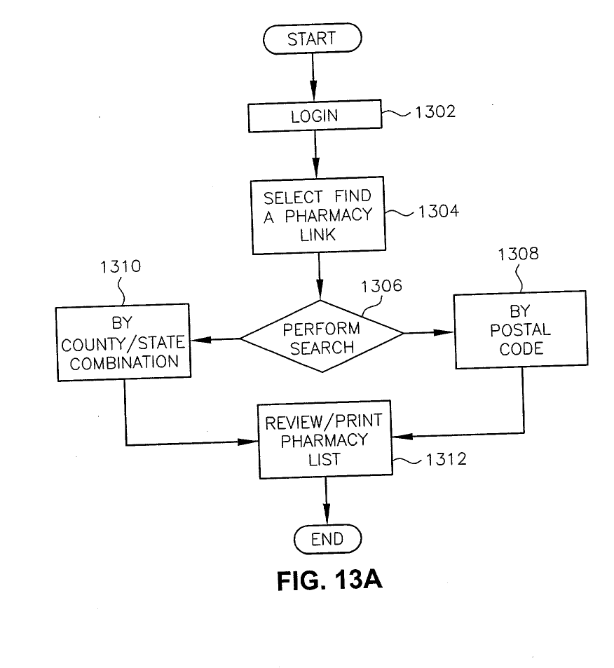

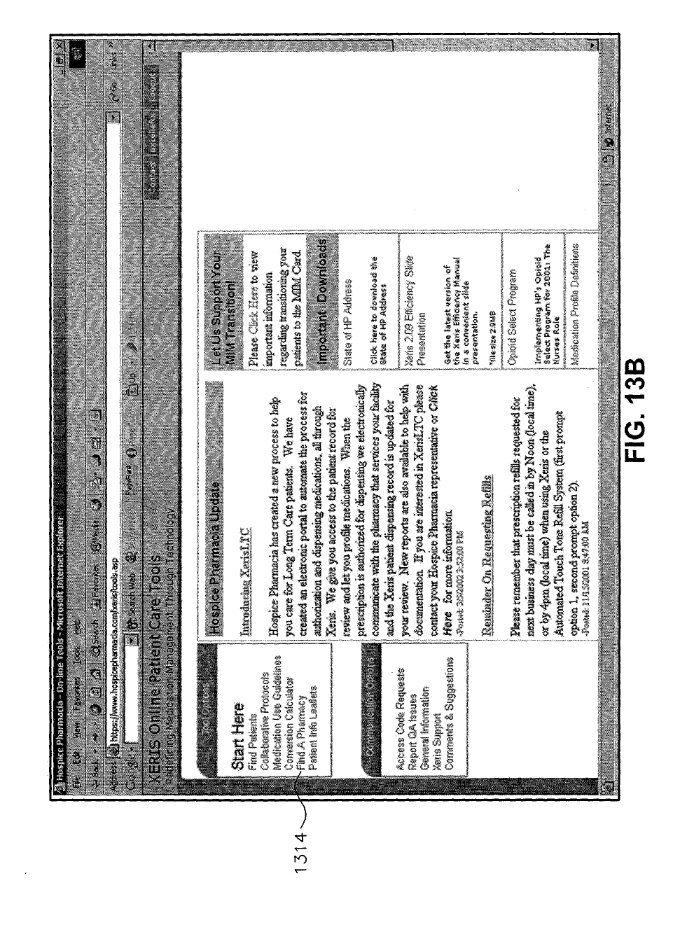

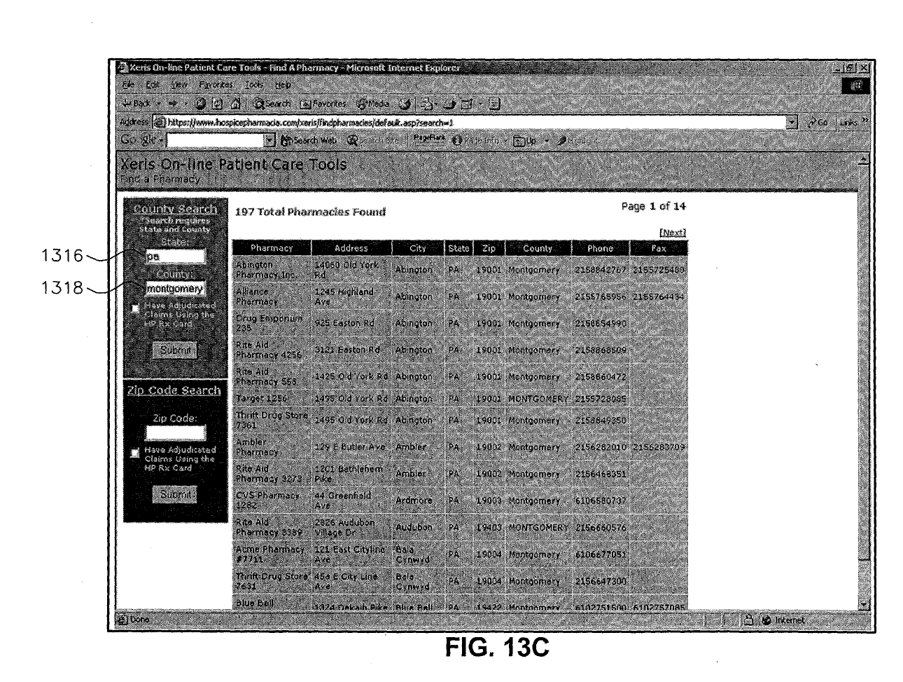

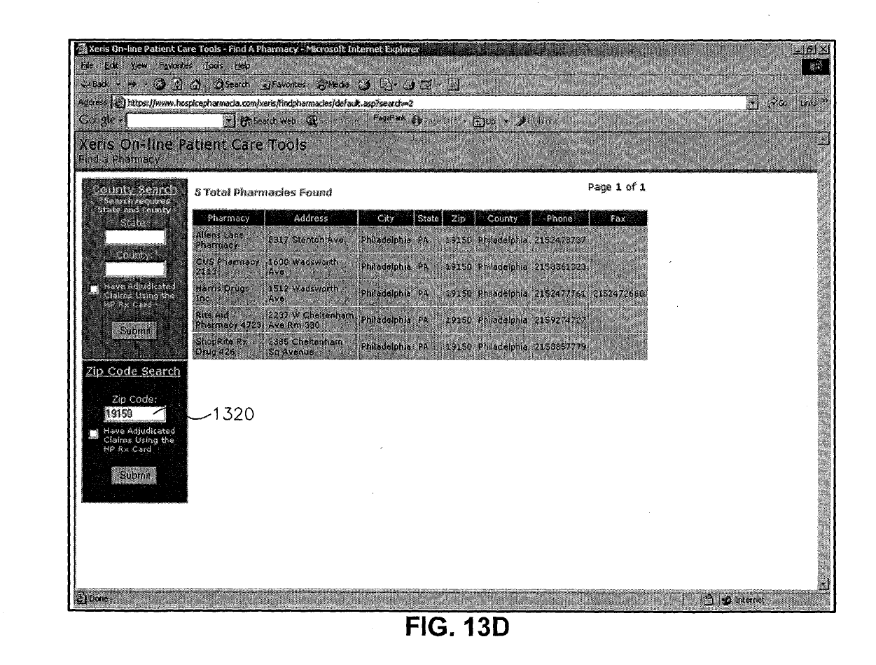

In one embodiment of the present invention, caregivers can use caregiver interface site 200 of network system 100 to locate a pharmacy. One embodiment of the process of locating a pharmacy using network system 100 is illustrated in a flowchart of FIG. 13A. As shown, a caregiver logs on to network system 100 in step 1302. From caregiver interface site 200, the caregiver may choose to find a pharmacy in step 1304. In step 1306, different ways of searching can be decided. For instance, a pharmacy can be searched using postal code, as shown in step 1308. Alternatively, a pharmacy can be searched by using a combination of county and state, as shown in step 1310. The result of this search containing a list of pharmacies can be reviewed and/or printed in step 1312.

Exemplary web pages illustrating caregiver interface site 200 that can be used by the caregiver to access network system 100 and find pharmacies as described above are shown in FIGS. 13B-13D. FIG. 13B shows one embodiment of caregiver interface site 200 that contains a link to “find a pharmacy” option. By clicking find a pharmacy link 1314, a caregiver is directed to one of caregiver interface site 200 that can be used to search the pharmacy. As noted earlier, a pharmacy can be searched by county and state combination or by zip code. The result of a search showing pharmacies found by looking up state and county combination is shown in FIG. 13C. As shown, in one embodiment of caregiver interface site 200 includes state box 1316 and county box 1318 for entering state and county information, respectively. Alternatively, the result of a search showing pharmacies found by looking up zip code is shown in FIG. 13D. As shown, the embodiment of caregiver interface site 200 further contains zip code box 1320 for zip code information. In one embodiment, the information obtained in the process of FIG. 13A is used to identify a pharmacy that will dispense medication on the same day as the decision to prescribe the medication is made by the caregiver. This allows the patient to receive same day service for acute conditions.

In one embodiment of the present invention, network system 100 is used to facilitate secure exchange of patient demographic data and medication profile information between and among main system 110 and various remote systems 140 used by hospices. In particular, data relating to prescriptions can be exchanged securely and effectively. Thus, using this feature, the errors caused by redundant re-entering of patient data is minimized, which in turn, increases efficiency.

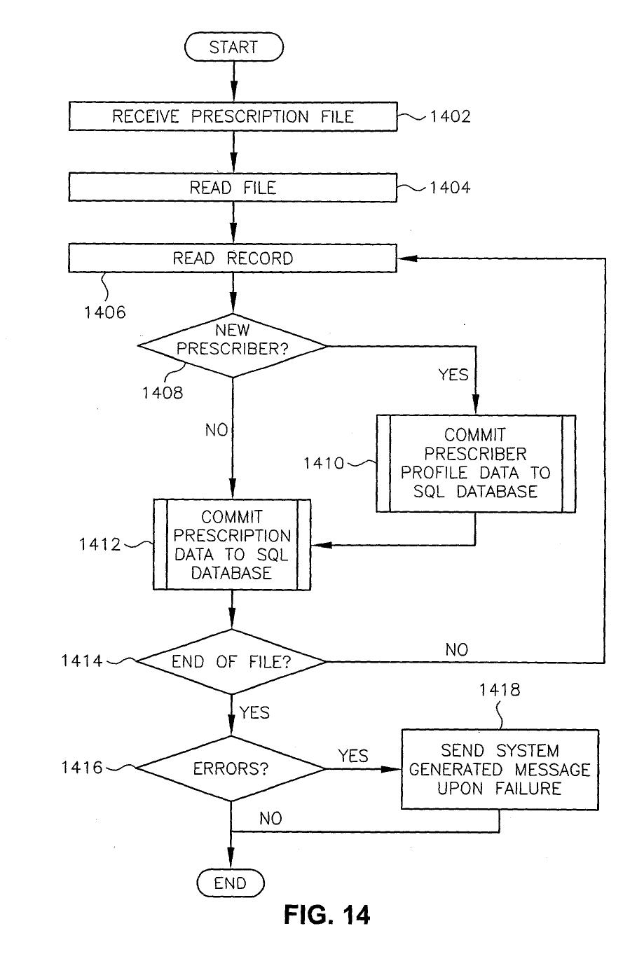

The system process of securely exchanging information using network system 100 is illustrated in FIG. 14. Using network system 100, a data center having main system 110 receives prescription files from a caregiver, as shown in step 1402. The prescription files are read in step 1404. All records of the prescription files are read in step 1406. In step 1408, the status of the caregiver is determined. That is, if the caregiver is not a new caregiver, the prescription data is sent to SQL database, as shown in step 1412. If, on the other hand, the caregiver is a new caregiver, a caregiver profile is first sent to SQL database, as shown in step 1410, and the prescription data is subsequently sent to SQL database in step 1412. As shown in step 1414, the process of reading begins again if there are more prescription files. If there no more files, all errors are checked in step 1416. If any error is discovered, a message is generated indicating to that effect in step 1418.

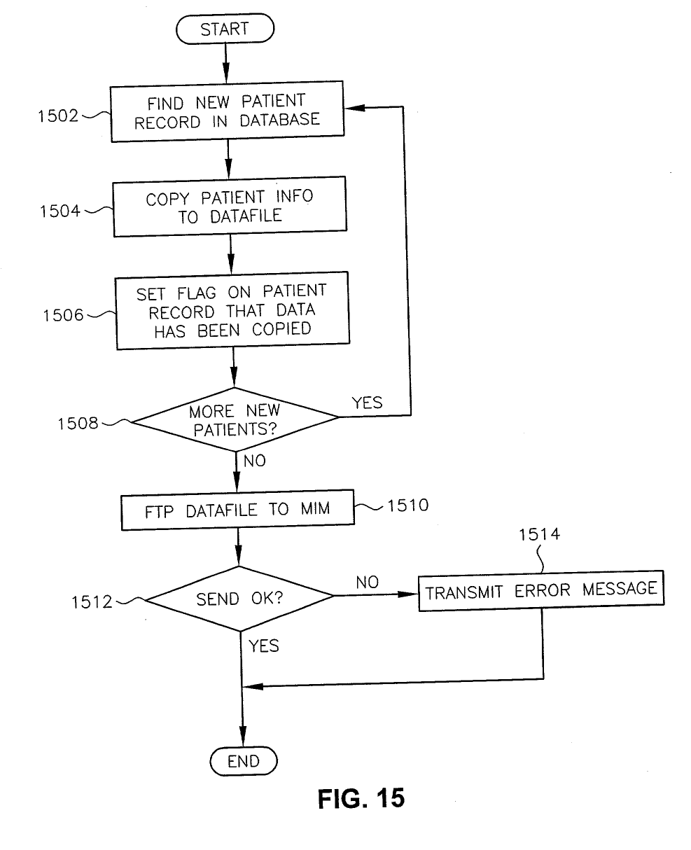

In one embodiment of the present invention, network system 100 is used to exchange patient information between a caregiver and a non-caregiver. For limited purposes, for instance, a third party may receive from network system 100 a certain information relating to patient profiles such as patient demographic information. The system process of exchanging certain patient information using network system 100 is illustrated in FIG. 15.

In step 1502, all new patient profiles in the database are accessed. Based on instructions or commands by an authorized caregiver, certain specific patient information is copied into a data file in step 1504. In step 1506, a flag, indicating on each patient record that the information has been copied, is set. Step 1508 determines whether there are more new patients. If there are more new patient profiles in the database, these need to be accessed again as in step 1502. In step 1510, if there are no more new patient profiles in the database, all new patient data that has been copied into the data file in step 1504 are transferred via FTP (File Transfer Protocol) to a third party. In step 1512, the transmission of data (i.e., FTP) is verified. If the data has not been transferred correctly, an error message is sent in step 1514.

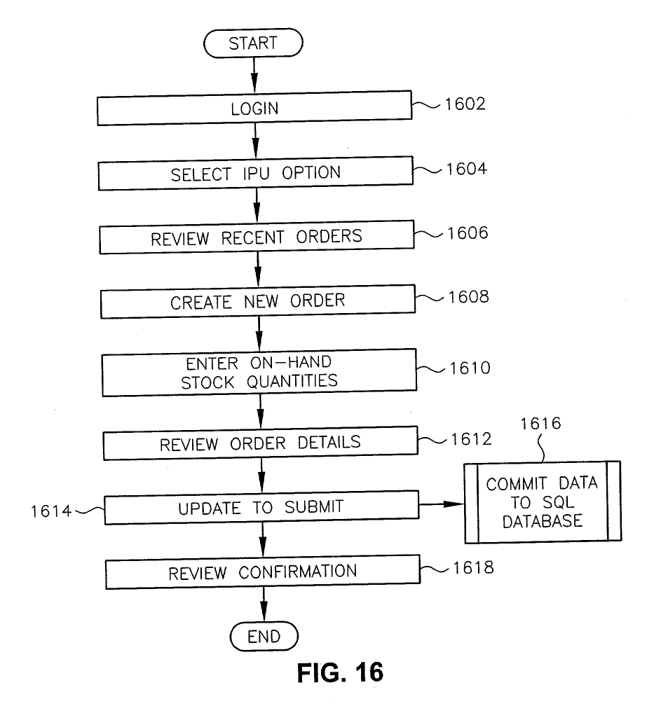

In one embodiment of the present invention, caregivers can use caregiver interface site 200 of network system 100 to place and/or review an order for medications in bulk. One embodiment of the process of placing and/or reviewing a bulk order or medication using network system 100 is illustrated in a flowchart of FIG. 16.

In step 1602, a caregiver logs in to network system 100 using network 10. The caregiver is directed to the present embodiment of caregiver interface site 200 in step 1604. The present embodiment of caregiver interface site 200 displays a list of the ten (10) most recent floor stock orders listed in reverse chronological order. In step 1606, the caregiver can either view these recent orders, or create a new order. In step 1608, the caregiver creates a new order. When creating a new order, the caregiver is presented with an order form listing the currently active medications that the In-Patient Unit (IPU) (i.e., a unit in a 24 hour care facility in which a higher degree of care is given to patients in need of such care) has agreed to stock based on the pharmacy systems currently active inventory. In step 1610, the caregiver then enters the IPU's current on-hand amounts for each medication on the form, and can either choose the proceed to check out, or cancel the order option.

The caregiver can review the order details in step 1612. Thereafter, in step 1614, the order entry entered is saved. Data representing this transaction is sent to SQL database, as shown in step 1616. In step 1618, the caregiver user can confirm the items by selecting process order, or remove a line item from the order details. Once the process order option is selected, the caregiver is presented with the order details list with an ordered status. The caregiver can then create additional orders, or logout of network system 100.

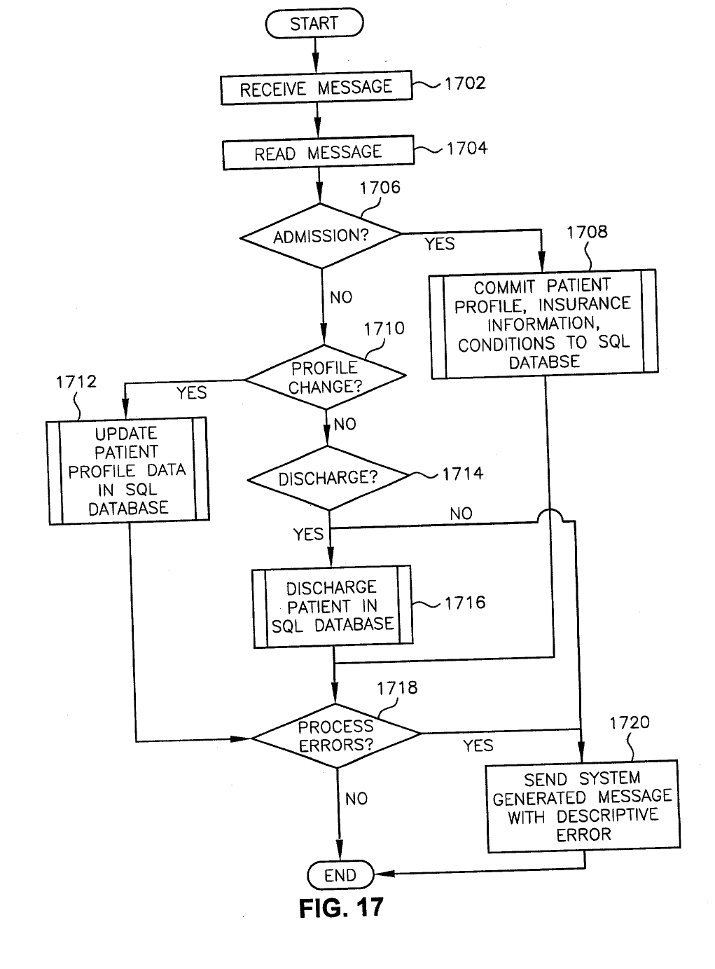

In one embodiment of the present invention, caregivers can use caregiver interface site 200 of network system 100 to interchange or access all pertinent information existing in main system 110 or remote system 140. Using the present embodiment of caregiver interface site 200, the caregivers can also interchange existing data between one another. In particular, the present embodiment allows the caregivers to send specific messages relating to their patients and/or medications associated with the patients thereof. One embodiment of the process for the caregivers to communicate with main system 110 or remote system 140 using network system 100 is illustrated in a flowchart of FIG. 17.

The process begins, in step 1702, where a message is received from a caregiver. Note that the message, in accordance with one aspect of the present invention, is encrypted through a proprietary messaging system to protect the confidentiality of the nature of the message. The message is read, in step 1704, to capture the data to determine the type of the message. There are three message types: an admission message, a profile change message, and a discharge message. Each message includes information relating to one or more patients. If the message is an admission message, which is determined in step 1706, an appropriate patient profile, insurance and medical conditions are committed to the SQL database in step 1708. If the message is a profile change message, which is determined in step 1710, the patient profile and medical conditions are committed to the SQL database in step 1712. If the message type is a discharge message, which is determined in step 1714, the discharge date is committed to the SQL database in step 1716.

As noted, a message may include information relating to more than one patient. If there are more patient records in the message, the process loops back to the beginning. If there are no more patient records within the message, the process checks to see whether there are more messages. Also, if there are additional messages, the process loops back to the beginning. When there are no additional messages, in step 1718, it is determined whether any errors were generated. If any errors exist, in step 1720, an error message is sent.

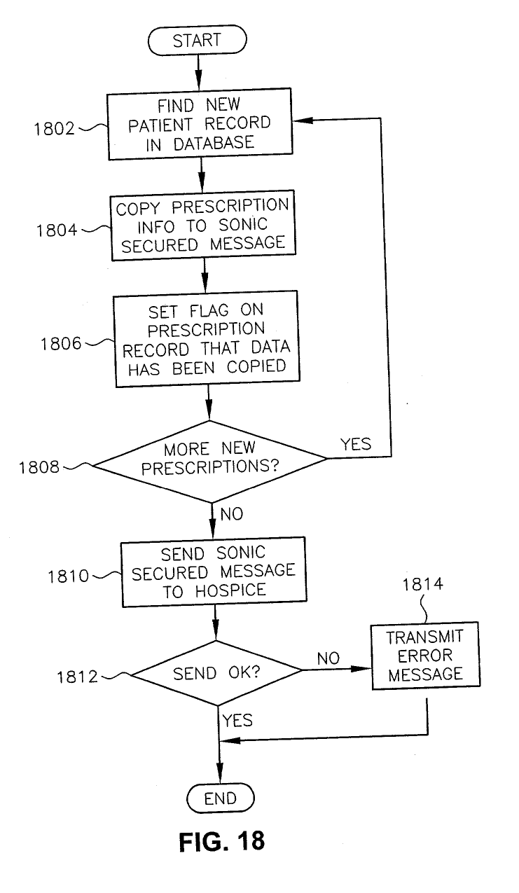

In one embodiment of the present invention, network system 100 is used to exchange medication information between caregivers. In particular, using network system 100, information relating to a specific prescription, including clinical data of the prescription, is exchanged to and from the caregivers. The system process of exchanging medical information, especially information relating to a specific prescription, using network system 100 is illustrated in FIG. 18.

In step 1802, all new prescription information in the database is accessed. Based on instructions or commands by an authorized caregiver, certain prescription information is copied into a secure messaging file in step 1804. In step 1806, a flag on each prescription record is set, indicating that the information has been copied. Step 1808 determines whether there are more new prescriptions. If there are more new prescription files in the database, these need to be accessed again as in step 1802. When all new prescription information copied into the secure messaging file, in step 1810, the messaging file is transmitted to a caregiver, such as a requesting hospice. In step 1812, any errors relating to the transmission of the messaging file is verified. If there are any errors, an error message is sent in step 1814.

While the description herein refers to the information existing in multiple databases, those of ordinary skill in the art will recognize and understand that all such information could be stored in a single database or in several databases structured differently than those described herein.

Furthermore, while much of the description herein regarding the systems and methods of the present invention pertains to the patients in a hospice setting, the systems and methods, in accordance with the present invention, are equally applicable to any health care system including a hospital, clinic, long-term-care facility, nursing home, patient's home, and may be used for inpatient and out-patient care.

It will be appreciated by those skilled in the art that changes could be made to the embodiments described above without departing from the broad inventive concept thereof. It is understood, therefore, that this invention is not limited to the particular embodiments disclosed, but is intended to cover modifications within the spirit and scope of the present invention as defined in the appended claims.

What is claimed is:

1. A method for selecting a medication therapy for a patient, comprising:
receiving input data relating to a patient profile for the patient, wherein the patient profile includes at least one of an objective attribute and a subjective attribute;
comparing the patient profile of the patient to an iterative patient database that includes a plurality of the patient profiles for a plurality of patients, each of the plurality of patients having at least one of the objective attribute and the subjective attribute that is similar to at least one of the objective attribute and the subjective attribute of the patient profile for the patient; and
selecting the medication therapy for the patient based on the comparing step.

2. The method of claim 1, wherein the comparing step comprises:
assessing results of administering the medication therapy to each of the plurality of patients having at least one of the objective attribute and the subjective attribute that is similar to at least one of the objective attribute and the subjective attribute of the patient profile for the patient; and
predicting outcomes of administering the medication therapy to the patient based on the results.

3. The method of claim 1, wherein the objective attribute includes at least one of a demographic profile and a medical history.

4. The method of claim 3, wherein the demographic profile includes at least one of a gender, race, age, geographic data, physical data, and financial data.

5. The method of claim 3, wherein the medical history includes at least one of an allergy, treating physician data, a lab result, medication data, a past symptom, a past diagnosis, a past medical indication, a hospital record, a hospice record, a pharmacy record, a current diagnosis, a current medical indication.

6. The method of claim 1, wherein the objective attribute includes pharmacogenomic data.

7. The method of claim 1, wherein the objective attribute includes at least one of ICD-9 primary code, ICD-9 primary term, ICD-9 secondary code, ICD-9 secondary term, and a clinical status of the patient.

8. The method of claim 1, wherein the subjective attribute includes at least one of a pain level and a level of personal satisfaction.

9. The method of claim 1, wherein the subjective attribute includes an individual's result of treatment using the medication therapy on the individual.

10. The method of claim 1, wherein the outcomes include the efficacy of the medication therapy.

11. A system for selecting a medication therapy of a patient, comprising:
an iterative database that stores a plurality of patient profiles for a plurality of patients, each of the plurality of patient profiles including at least one of an objective attribute and a subjective attribute; and
a processor that receives input data relating to the patient profile of the patient, compares the patient profile including at least one of the objective attribute and the subjective attribute to a subset of the plurality of patient profiles including at least one of the objective attribute and the subjective attribute that is similar to at least one of the objective attribute and the subjective attribute of the patient profile, and selects the medication therapy for the patient based on the comparison.

12. The system of claim 11, wherein the objective attribute includes at least one of a demographic profile, and a medical history.

13. The system of claim 12, wherein the demographic profile includes at least one of a gender, race, age, geographic data, physical data, and financial data.

14. The system of claim 12, wherein the medical history includes at least one of an allergy, treating physician data, a lab result, medication data, a past symptom, a past diagnosis, a past medical indication, a hospital record, a hospice record, a pharmacy record, a current diagnosis, a current medical indication.

15. The system of claim 11, wherein the objective attribute includes pharmacogenomic data.

16. The system of claim 11, wherein the objective attribute includes at least one of ICD-9 primary code, ICD-9 primary term, ICD-9 secondary code, ICD-9 secondary term, and a clinical status of the patient.

17. The system of claim 11, wherein the subjective attribute includes at least one of a pain level and a level of personal satisfaction.

18. The system of claim 11, wherein the subjective attribute includes an individual's result of treatment using the medication therapy on the individual.

19. The system of claim 11, wherein the outcomes include the efficacy of the medication therapy.

20. A network system for selecting and distributing a medication therapy for a patient, the system comprising:
an iterative database that stores a plurality of patient profiles for a plurality of patients, each of the plurality of patient profiles including at least one of an objective attribute and a subjective attribute;
a wireless device that transmits input data including at least one of the objective attribute and the subjective attribute for the patient profile of the patient, and receives output data;
a processor that receives the input data including the profile of the patient, compares the patient profile of the patient to a subset of the plurality of patient profiles, each of the patient profile from the subset having at least one of the objective attribute and the subjective attribute that is similar to at least one of the objective attribute and the subjective attribute of the patient profile of the patient, selects the medication therapy for the patient based on the comparing step, and transmits the output data that includes the medication therapy for the patient.

21. The system of claim 20, wherein the objective attribute includes at least one of a demographic profile, and a medical history.

22. The system of claim 21, wherein the demographic profile includes at least one of a gender, race, age, geographic data, physical data, and financial data.

23. The system of claim 21, wherein the medical history includes at least one of an allergy, treating physician data, a lab result, medication data, a past symptom, a past diagnosis, a past medical indication, a hospital record, a hospice record, a pharmacy record, a current diagnosis, a current medical indication.

24. The system of claim 20, wherein the objective attribute includes pharmacogenomic data.

25. The system of claim 20, wherein the objective attribute includes at least one of ICD-9 primary code, ICD-9 primary term, ICD-9 secondary code, ICD-9 secondary term, and a clinical status of the patient.

26. The system of claim 20, wherein the subjective attribute includes at least one of a pain level and a level of personal satisfaction.

27. The system of claim 20, wherein the subjective attribute includes an individual's result of treatment using the medication therapy on the individual.

28. The system of claim 20, wherein the outcomes include the efficacy of the medication therapy.

29. The system of claim 20, wherein the wireless device comprises a Personal Digital Assistant.

30. The system of claim 20, wherein the wireless device comprises a hand-held computer.

31. A method for selecting a medication therapy for a patient, comprising:
presenting a graphical user interface to a caregiver of the patient;
receiving via the graphical user interface input data relating to a patient profile of the patient from the caregiver, the patient profile including at least one of an objective attribute and a subjective attribute;
comparing the patient profile of the patient to patient data that includes a plurality of the patient profiles for a plurality of patients, each of the patient profiles including at least one of the objective attribute and the subjective attribute that is similar to at least one of the objective attribute and the subjective attribute of the patient;
predicting outcomes of administering the medication therapy for the patient based on the comparing step; and
displaying the outcomes to the caregiver via the graphical user interface.

32. The method of claim 31, wherein the objective attribute includes at least one of a demographic profile, and a medical history.

33. The method of claim 32, wherein the demographic profile includes at least one of a gender, race, age, geographic data, physical data, and financial data.

34. The method of claim 32, wherein the medical history includes at least one of an allergy, treating physician data, a lab result, medication data, a past symptom, a past diagnosis, a past medical indication, a hospital record, a hospice record, a pharmacy record, a current diagnosis, a current medical indication.

35. The method of claim 31, wherein the objective attribute includes pharmacogenomic data.

36. The method of claim 31, wherein the objective attribute includes at least one of ICD-9 primary code, ICD-9 primary term, ICD-9 secondary code, ICD-9 secondary term, and a clinical status of the patient.

37. The method of claim 31, wherein the subjective attribute includes at least one of a pain level and a level of personal satisfaction.

38. The method of claim 31, wherein the subjective attribute includes an individual's result of treatment using the medication therapy on the individual.

39. The method of claim 31, wherein the outcomes include the efficacy of the medication therapy.

40. The method of claim 31, wherein the caregiver includes at least one from the group of a prescriber, a physician, a nurse, a pharmacist, a hospice caretaker, and the patient.
